(19) United States
(12) Patent Application Publication
(10) Pub. No.: US 2025/0057547 A1
(43) Pub. Date: Feb. 20, 2025

(54) APPARATUS, SYSTEM, AND METHOD FOR PATIENT-SPECIFIC SYSTEMS, METHODS, AND INSTRUMENTATION

(71) Applicant: Treace Medical Concepts, Inc., Ponte Vedra, FL (US)

(72) Inventors: James Q. Spitler, Winter Garden, FL (US); Lee M. Hlad, Waycross, GA (US)

(21) Appl. No.: 18/808,935

(22) Filed: Aug. 19, 2024

Related U.S. Application Data
(60) Provisional application No. 63/533,554, filed on Aug. 18, 2023.

(51) Int. Cl. A61B 17/17; A61B 17/15; A61B 17/56; A61B 34/10

(52) U.S. Cl. CPC A61B 17/1775; A61B 17/152; A61B 34/10; A61B 2017/565; A61B 2034/105; A61B 2034/107

(57) ABSTRACT

An apparatus, system, and method are disclosed for remediating a condition present in a patient. In some implementations, a resection guide includes a first resection feature that guides a cutting tool to form a first osteotomy in a bone, the first resection feature extends along a first trajectory. A second resection feature guides a cutting tool to form a second osteotomy in the bone, the second resection feature extends along a second trajectory. A third resection feature guides a cutting tool to form a third osteotomy in the bone, the third resection feature extends along a third trajectory. At least one of the trajectories is determined based on a bone model of a portion of the bone, which bone model is based on medical imaging of the patient's foot. The device also includes a bone attachment feature to secure the resection guide to the bone.

### CROSS-REFERENCE TO RELATED APPLICATIONS

This application claims the benefit of U.S. Provisional Application No. 63/533,554, filed Aug. 18, 2023, which is hereby incorporated by reference in its entirety.

### TECHNICAL FIELD

The present disclosure relates to surgical devices, systems, instruments, and methods. More specifically, the present disclosure relates to patient-specific instruments, implants, instruments, and/or methods of designing and using the same.

### BACKGROUND

Various bone conditions may be corrected using surgical procedures, in which one or more tendons, ligaments, and/or bones may be cut, replaced, repositioned, reoriented, reattached, fixated and/or fused. These surgical procedures require the surgeon to accurately locate, position, deploy, and/or orient one or more osteotomy cuts, fixation guides, fixators, bone tunnels, implants, points of attachment for ends of grafts or soft tissue, and the like. Determining and locating an optimal location and trajectory for one or more steps of the surgical procedures and/or securing instruments that can guide or assist in steps of the surgical procedures such as performing osteotomies, deploying fixation and/or implants, and the like, can be challenging, given conventional techniques and instruments.

One of the challenges with conventional techniques is how to translate, map, or convert from a model of a patient's anatomy and/or virtual instrumentation to the real, physical world for performing a surgical procedure. Furthermore, surgical procedures can be extra challenging when working on anatomy such as bones of a patient's ankle, foot, or hand which are small in size, have unique surface configurations, landmarks, and/or deformities that called for extra accuracy and/or precision. In certain surgical procedures such as a joint fusion, one goal may be to minimize the amount of bone removed in order to successfully fuse the joint. Accomplishing this goal can require extra precision and accuracy in resecting the bone(s), reducing the bones, and/or deploying fixation to achieve a successful fusion.

### SUMMARY

Some implementations of the present disclosure relate to a patient-specific apparatus. For example, one apparatus may include a resection guide having: a body having an anterior side, a posterior side, a medial side, a lateral side, a superior side, and an inferior side; a first resection feature configured to guide a cutting tool to form a first osteotomy in a bone, the first resection feature extending through the resection guide from the medial side to the lateral side along a first trajectory; a second resection feature configured to guide a cutting tool to form a second osteotomy in the bone, the second resection feature extending through the resection guide from the medial side to the lateral side along a second trajectory; a third resection feature configured to guide a cutting tool to form a third osteotomy in the bone, the third resection feature extending through the resection guide from the medial side to the lateral side along a third trajectory. At least one of the first trajectory, the second trajectory, and the third trajectory are at least partially determined based on a bone model of at least a portion of the bone, the bone model based on medical imaging of the patient's foot and configured to resemble an anatomy of the patient's foot; and a bone attachment feature configured to secure the resection guide to the bone.

The described implementations may also include one or more of the following features. An apparatus where the third resection feature intersects at least one of the first resection feature and the second resection feature at an intersection angle of between about 45 and about 135 degrees. An apparatus where the third resection feature intersects both the first resection feature and the second resection feature at an intersection angle of about 90 degrees. An apparatus where the first trajectory converges with the second trajectory at a vertex having a wedge angle, and the third trajectory intersects the first trajectory and the second trajectory to form a wedge osteotomy, the wedge angle determined based, at least in part, on the bone model. An apparatus where the vertex is prepositioned to be between a lateral cortex of the bone and the resection guide when the resection guide is designed for the bone. An apparatus where the vertex is prepositioned such that an osteotomy formed in two of the resection features forms a living hinge at a cortex of the bone. An apparatus where the resection guide may include: a bone engagement feature configured to engage with at least a portion of the bone at a position that substantially matches a model position of a model of the resection guide engaging the bone model. An apparatus where the bone engagement feature may include: a bone engagement surface configured to at least partially match a contour of a surface of the bone when the resection guide is positioned for use; and where the bone engagement surface is on one of a lateral side and a medial side of the body. An apparatus where the bone engagement surface registers with the surface of the bone abutting the resection guide when the resection guide is positioned for use. An apparatus where the bone attachment feature forms at least one hole in the bone, the at least one hole configured to serve as an anchor hole for a fixation device. An apparatus where the third resection feature may include a closed end and an open end.

Some implementations herein relate to an apparatus. For example, an apparatus may include a resection guide having: a body having an anterior side, a posterior side, a medial side, a lateral side, a superior side, and an inferior side; a resection feature configured to guide a cutting tool to form a wedge osteotomy in a metatarsal, the wedge osteotomy forming a wedge bone fragment, the wedge osteotomy at least partially determined based on a bone model of at least a portion of the metatarsal; and a bone attachment feature configured to secure the resection guide to the bone.

The described implementations may also include one or more of the following features. An apparatus where the resection feature is configured to form a shelf in a head of the metatarsal. An apparatus where the resection feature is configured to form a planar surface on a proximal side of the wedge osteotomy, the planar surface configured to engage with the shelf during fusion to provide increased bone contact for fusion of the wedge osteotomy. An apparatus where the resection guide may include: a bone engagement surface configured to at least partially match a contour of a surface of the metatarsal when the resection guide is positioned for use; and where the bone engagement surface is on a lateral side of the body. An apparatus where the wedge osteotomy may include an osteotomy parallel to a plantar weight bearing surface of the metatarsal, proximal to a distal articular surface and superior to a plantar aspect of a metatarsal head of the metatarsal. An apparatus where the resection feature may include: a first section extending through the resection guide from the medial side to the lateral side, the first section having: a first leg extending through the resection guide along a first trajectory; a second leg extending through the resection guide along a second trajectory; and a second section extending through the resection guide from the medial side to the lateral side, the second section intersecting the first leg at a first turn having a first intersection angle and the second leg at a second turn having a second intersection angle, the second section extending through the resection guide along a third trajectory. An apparatus where the second section is positioned within the resection guide such that the second section protects sesamoids that are plantar to a head of the metatarsal from dissection.

Some implementations herein relate to a method. For example, a method may include positioning a resection guide onto a medial surface of a distal end of a metatarsal, the resection guide having: a body having an anterior side, a posterior side, a medial side, a lateral side, a superior side, and an inferior side; a first resection feature configured to guide a cutting tool to form a first osteotomy in a metatarsal, the first resection feature extending through the resection guide from the medial side to the lateral side along a first trajectory at least partially determined based on a bone model of at least a portion of the metatarsal, the bone model based on medical imaging of the patient's foot and configured to resemble an anatomy of the patient's foot; a second resection feature configured to guide a cutting tool to form a second osteotomy in the metatarsal, the second resection feature extending through the resection guide from the medial side to the lateral side along a second trajectory at least partially determined based on the bone model; a third resection feature configured to guide a cutting tool to form a third osteotomy in the metatarsal, the third resection feature extending through the resection guide from the medial side to the lateral side along a third trajectory at least partially determined based on the bone model; and a bone attachment feature configured to secure the resection guide to the metatarsal. A method may also include deploying a set of fasteners as part of the bone attachment feature to secure the resection guide to the metatarsal. A method may furthermore include inserting the cutting tool into the first resection feature, second resection feature, and third resection feature and cutting the metatarsal to form one or more osteotomies of a Reverdin procedure. A method may in addition include deploying fixation hardware across one or more of the osteotomies to enable fusion of the metatarsal. Other embodiments of this aspect include corresponding computer systems, apparatus, and computer programs recorded on one or more computer storage devices, each configured to perform the actions of the methods.

The described implementations may also include one or more of the following features. Method where the Reverdin procedure may include one of: a Reverdin-Green procedure, a Reverdin-Laird procedure; and a Reverdin-Todd procedure.

Other embodiments of the present disclosure may include corresponding computer systems, apparatuses, and computer programs recorded on one or more computer storage devices, each configured to perform the actions of the methods. Implementations of the described techniques may include hardware, a method or process, or a computer tangible medium.

### BRIEF DESCRIPTION OF THE DRAWINGS

The advantages, nature, and additional features of exemplary embodiments of the disclosure will become more fully apparent from the following description and appended claims, taken in conjunction with the accompanying drawings. Understanding that these drawings depict only exemplary embodiments and are, therefore, not to be considered limiting of the disclosure's scope, the exemplary embodiments of the disclosure will be described with additional specificity and detail through use of the accompanying drawings.

FIG. 1A is a flowchart diagram depicting a method for remediating a condition, according to one embodiment.

FIG. 1B is a flowchart diagram depicting a method for remediating a condition, according to one embodiment.

FIG. 2A is a dorsal perspective view of bones of a foot.

FIG. 2B is a lateral perspective view of bones of a foot.

FIG. 2C is a medial perspective view of bones of a foot.

FIG. 2D is a dorsal perspective view of bones of a foot.

FIG. 2E is a view of a foot illustrating common planes of reference for a human foot.

FIG. 3 is a flowchart diagram depicting a method for generating one or more patient-specific instruments, according to one embodiment.

FIG. 4 illustrates an exemplary system configured to generate one or more patient-specific instruments, according to one embodiment.

FIG. 5 illustrates an exemplary system configured to generate one or more patient-specific instruments, according to one embodiment.

FIG. 6 illustrates an exemplary system configured to generate a patient-specific osteotomy system, according to one embodiment.

FIG. 7 illustrates an exemplary system for remediating a condition present in a patient's foot, according to one embodiment.

FIG. 8 illustrates exemplary bones of a patient with a bone condition suitable for use with an apparatus, system, and/or method of the present disclosure, according to one embodiment.

FIG. 9 illustrates an exemplary system for an osteotomy, according to one embodiment.

FIGS. 10A-10G illustrate views of a resection guide of the osteotomy system of FIG. 9, according to one embodiment.

FIG. 10H illustrates a cross-section view of the resection guide of FIG. 10A taken along line 10H, according to one embodiment.

FIG. 10I illustrates a cross-section view of the resection guide in FIG. 10C taken along line 10I, according to one embodiment.

FIGS. 11A-11E illustrate views of a resection guide of an osteotomy system, according to one or more embodiments.

FIG. 12 is a flowchart diagram depicting a method for remediating a bone condition, according to one embodiment.

FIGS. 13A-13C illustrate different views of a surgical osteotomy procedure using the osteotomy system of FIG. 9, according to one embodiment.

FIG. 13D illustrates a closeup view of a resection guide positioned on a metatarsal after forming one or more osteotomies.

FIG. 13E illustrates a closeup view of one or more osteotomies near a metatarsal head formed using a resection guide according to one embodiment.

FIG. 13F illustrates a reduced and fixated distal metatarsal head after one or more osteotomies using a resection guide according to one embodiment.

### DETAILED DESCRIPTION

Exemplary embodiments of the disclosure will be best understood by reference to the drawings, wherein like parts are designated by like numerals throughout. It will be readily understood that the components, as generally described and illustrated in the Figures herein, could be arranged and designed in a wide variety of different configurations. Thus, the following more detailed description of the embodiments of the apparatus, system, and method is not intended to limit the scope of the disclosure but is merely representative of exemplary embodiments.

The phrases “connected to,” “coupled to” and “in communication with” refer to any form of interaction between two or more entities, including mechanical, electrical, magnetic, electromagnetic, fluid, and thermal interaction. Two components may be functionally coupled to each other even though they are not in direct contact with each other. The term “abutting” refers to items that are in direct physical contact with each other, although the items may not necessarily be attached together. The phrase “fluid communication” refers to two features that are connected such that a fluid within one feature can pass into the other feature.

The word “exemplary” is used herein to mean “serving as an example, instance, or illustration.” Any embodiment described herein as “exemplary” is not necessarily to be construed as preferred or advantageous over other embodiments. While the various aspects of the embodiments are presented in drawings, the drawings are not necessarily drawn to scale unless specifically indicated.

Standard medical planes of reference and descriptive terminology are employed in this disclosure. While these terms are commonly used to refer to the human body, certain terms are applicable to physical objects in general. A standard system of three mutually perpendicular reference planes is employed. A sagittal plane divides a body into right and left portions. A coronal plane divides a body into anterior and posterior portions. A transverse plane divides a body into superior and inferior portions. A mid-sagittal, mid-coronal, or mid-transverse plane divides a body into equal portions, which may be bilaterally symmetric. The intersection of the sagittal and coronal planes defines a superior-inferior or cephalad-caudal axis. The intersection of the sagittal and transverse planes defines an anterior-posterior axis. The intersection of the coronal and transverse planes defines a medial-lateral axis. The superior-inferior or cephalad-caudal axis, the anterior-posterior axis, and the medial-lateral axis are mutually perpendicular.

Anterior means toward the front of a body. Posterior means toward the back of a body. Superior or cephalad means toward the head. Inferior or caudal means toward the feet or tail. Medial means toward the midline of a body, particularly toward a plane of bilateral symmetry of the body. Lateral means away from the midline of a body or away from a plane of bilateral symmetry of the body. Axial means toward a central axis of a body. Abaxial means away from a central axis of a body. Ipsilateral means on the same side of the body. Contralateral means on the opposite side of the body from the side which has a particular condition or structure. Proximal means toward the trunk of the body. Proximal may also mean toward a user, viewer, or operator. Distal means away from the trunk. Distal may also mean away from a user, viewer, or operator. Dorsal means toward the top of the foot or other body structure. Plantar means toward the sole of the foot or toward the bottom of the body structure.

Antegrade means forward moving from a proximal location/position to a distal location/position or moving in a forward direction. Retrograde means backward moving from a distal location/position to a proximal location/position or moving in a backwards direction. Sagittal refers to a midline of a patient's anatomy, which divides the body into left or right halves. The sagittal plane may be in the center of the body, splitting it into two halves. Prone means a body of a person lying face down. Supine means a body of a person lying face up.

As used herein, “coupling”, “coupling member”, or “coupler” refers to a mechanical device, apparatus, member, component, system, assembly, or structure, that is organized, configured, designed, arranged, or engineered to connect, or facilitate the connection of, two or more parts, objects, or structures. In certain embodiments, a coupling can connect adjacent parts or objects at their ends. In certain embodiments, a coupling can be used to connect two shafts together at their ends for the purpose of transmitting power. In other embodiments, a coupling can be used to join two pieces of rotating equipment while permitting some degree of misalignment or end movement or both. In certain embodiments, couplings may not allow disconnection of the two parts, such as shafts during operation. (Search “coupling” on Wikipedia.com Jul. 26, 2021. CC-BY-SA 3.0 Modified. Accessed Jul. 27, 2021.) A coupler may be flexible, semiflexible, pliable, elastic, or rigid. A coupler may join two structures either directly by connecting directly to one structure and/or directly to the other or indirectly by connecting indirectly (by way of one or more intermediary structures) to one structure, to the other structure, or to both structures.

“Patient specific” refers to a feature, an attribute, a characteristic, a structure, function, structure, device, guide, tool, instrument, apparatus, member, component, system, assembly, module, or subsystem or the like that is adjusted, tailored, modified, organized, configured, designed, arranged, engineered, and/or fabricated to specifically address the anatomy, physiology, condition, abnormalities, needs, or desires of a particular patient or surgeon serving the particular patient. In one aspect, a patient specific attribute or feature is unique to a single patient and may include features unique to the patient such as a number of cut channels, a number of bone attachment features, a number of bone engagement surfaces, a number of resection features, a depth of one or more cutting channels, an angle for one or more resection channels, a surface contour, component position, component orientation, a trajectory for an instrument, implant, or anatomical part of a patient, a lateral offset, and/or other features.

“Patient-specific instrument” refers to an instrument, implant, or guide designed, engineered, and/or fabricated for use with a specific patient. In one aspect, a patient-specific instrument is unique to a patient and may include features unique to the patient such as a surface contour or other features.

“Patient-specific positioning guide” or “Patient-specific positioner” refers to an instrument, implant, positioner, structure, or guide designed, engineered, and/or fabricated for use as a positioner with a specific patient. In one aspect, a patient-specific positioning guide is unique to a patient and may include features unique to the patient such as patient-specific offsets, translation distances, openings, angles, orientations, anchor a surface contour or other features.

“Patient-specific cutting guide” refers to a cutting guide designed, engineered, and/or fabricated for use with a specific patient. In one aspect, a patient-specific cutting guide is unique to a patient and may include features unique to the patient such as a surface contour or other features.

“Patient-specific resection guide” refers to a guide designed, engineered, and/or fabricated for use in resection for a specific patient. In one aspect, a patient-specific resection guide is unique to a patient and may include features unique to the patient such as a surface contour or other features.

“Patient-specific trajectory guide” refers to a trajectory guide designed, engineered, and/or fabricated for use with a specific patient. In one aspect, a patient-specific trajectory guide is unique to a single patient and may include features unique to the patient such as a surface contour or other features.

“Patient specific instrument” (PSI) refers to a structure, device, guide, tool, instrument, apparatus, member, component, system, assembly, module, or subsystem that is adjusted, tailored, modified, organized, configured, designed, arranged, engineered, and/or fabricated to specifically address the anatomy, physiology, condition, abnormalities, needs, or desires of a particular patient. In certain aspects, one patient. In one aspect, a patient specific instrument is unique to a single patient and may include features unique to the patient such as a surface contour, component position, component orientation, and/or other features. In other aspects, one patient specific instrument may be useable with a number of patients having a particular class of characteristics.

As used herein, an “indicator” refers to an apparatus, device, component, system, assembly, mechanism, hardware, software, firmware, circuit, module, set of data, text, number, code, symbol, a mark, or logic structured, organized, configured, programmed, designed, arranged, or engineered to convey information or indicate a state, condition, mode, context, location, or position to another apparatus, device, component, system, assembly, mechanism, hardware, software, firmware, circuit, module, and/or a user of an apparatus, device, component, system, assembly, mechanism, hardware, software, firmware, circuit, module that includes, or is associated with the indicator. The indicator can include one or more of an audible signal, a token, a presence of a signal, an absence of a signal, a tactile signal, a visual signal or indication, a visual marker, a visual icon, a visual symbol, a visual code, a visual mark, and/or the like. In certain embodiments, “indicator” can be used with an adjective describing the indicator. For example, a “mode indicator” is an indicator that identifies or indicates a mode.

As used herein, a “handle” or “knob” refers to a structure used to hold, control, or manipulate a device, apparatus, component, tool, or the like. A “handle” may be designed to be grasped and/or held using one or two hands of a user. In certain embodiments, a handle or knob may be an elongated structure. In one embodiment, a knob may be a shorter stubby structure.

As used herein, “implant” refers to a medical device manufactured to replace a missing biological structure, support a damaged biological structure, or enhance an existing biological structure. Often medical implants are man-made devices, but implants can also be natural occurring structures. The surface of implants that contact the body may be made of, or include a biomedical material such as titanium, cobalt chrome, stainless steel, carbon fiber, another metallic alloy, silicone, polymer, Synthetic polyvinyl alcohol (PVA) hydrogels, biomaterials, biocompatible polymers such as PolyEther Ether Ketone (PEEK) or a polylactide polymer (e.g. PLLA) and/or others, or apatite, or any combination of these depending on what is functional and/or economical. Implants can have a variety of configurations and can be wholly, partially, and/or include a number of components that are flexible, semiflexible, pliable, elastic, supple, semi-rigid, or rigid. In some cases implants contain electronics, e.g. artificial pacemaker and cochlear implants. Some implants are bioactive, such as subcutaneous drug delivery devices in the form of implantable pills or drug-eluting stents. Orthopedic implants may be used to alleviate issues with bones and/or joints of a patient's body. Orthopedic implants can be used to treat bone fractures, osteoarthritis, scoliosis, spinal stenosis, discomfort, and pain. Examples of orthopedic implants include, but are not limited to, a wide variety of pins, rods, screws, anchors, spacers, sutures, all-suture implants, ball all-suture implants, self-locking suture implants, cross-threaded suture implants, plates used to anchor fractured bones while the bones heal or fuse together, and the like. (Search “implant (medicine)” on Wikipedia.com May 26, 2021. CC-BY-SA 3.0 Modified. Accessed Jun. 30, 2021.)

As used herein, a “body” refers to a main or central part of a structure. The body may serve as a structural component to connect, interconnect, surround, enclose, and/or protect one or more other structural components. A body may be made from a variety of materials including, but not limited to, metal, plastic, ceramic, wood, fiberglass, acrylic, carbon, biocompatible materials, biodegradable materials or the like. A body may be formed of any biocompatible materials, including but not limited to biocompatible metals such as Titanium, Titanium alloys, stainless steel alloys, cobalt-chromium steel alloys, nickel-titanium alloys, shape memory alloys such as Nitinol, biocompatible ceramics, and biocompatible polymers such as Polyether ether ketone (PEEK) or a polylactide polymer (e.g. PLLA) and/or others. In one embodiment, a body may include a housing or frame or framework for a larger system, component, structure, or device. A body may include a modifier that identifies a particular function, location, orientation, operation, and/or a particular structure relating to the body. Examples of such modifiers applied to a body, include, but are not limited to, “inferior body,” “superior body,” “lateral body,” “medial body,” and the like.

As used herein, “bone engagement surface” refers to a surface of an object, instrument, or apparatus, such as an implant that is oriented toward or faces one or more bones of a patient. In one aspect, the bone engagement surface may abut, touch, or contact a surface of a bone. In another aspect, the bone engagement surface or parts of the bone engagement surface may be close to, but not abut, touch, or contact a surface of the bone. In certain aspects, the bone engagement surface can be configured to engage with a surface of one or more bones. Such a bone engagement surface may include projections and recesses that correspond to and match projections and recesses of the one or more bone surfaces.

“Bone engagement feature” refers to a structure, feature, component, aspect configured to contact, touch, abut, and/or engage with a bone, a bone part, and/or a bone fragment. A bone engagement feature may enable temporary engagement with a bone or bone fragment or permanent engagement with a bone or bone fragment. A bone engagement feature may include a bone engagement surface and a body section that supports the bone engagement surface. In certain embodiments, a bone engagement feature may include a bone probe. In one embodiment, a bone engagement feature may include a landmark registration feature.

“Frangible” refers to a type of material designed, engineered, and/or configured to break easily under an expected force. Frangible objects may be designed to break easily under the expected force to provide a safety feature, a convenience feature, or the like. Frangible objects can be made from metal, plastic, ceramics, wood, paper, or the like. Frangible also includes something that is breakable or fragile; especially something that is intentionally made so. (Search “frangible” on wordhippo.com. WordHippo, 2023. Web. Accessed 11 May 2023. Modified.)

As used herein, “side” refers to a structure or part of a structure including, but not limited to: one of a longer bounding surfaces or lines of an object especially contrasted with the ends, a line or surface forming a border or face of an object, either surface of a thin object, a bounding line or structure of a geometric figure or shape, and the like. (search “side” on Merriam-Webster.com. Merriam-Webster, 2021. Web. 3 Aug. 2021. Modified.) A side can also refer to a geometric edge of a polygon (two-dimensional shape) and/or a face or surface of a polyhedron (three-dimensional shape). (Search “side” on Wikipedia.com Jul. 21, 2021. CC-BY-SA 3.0 Modified. Accessed Aug. 3, 2021.) Side can also refer to a location on a structure. For example, a side can be a location on a structure at, or near, a furthest position away from a central axis of the structure. As used herein, the term “side” can include one or more modifiers that define and/or orient and/or distinguish the side of an object from others based on based on where and/or how the object is deployed within or in relation to a second object. For example, in the context of an implant for a patient, sides of the implant may be labeled based on where the sides are relative to the patient when the implant is deployed. As one example, an “anterior side” of an implant, instrument, anatomical structure, or other structure refers to a side that is anterior to other sides of the structure in relation to a patient when the structure is deployed in the patient. As another example, in the context of an instrument used with a patient, sides of the instrument may be labeled based on where the sides are when the instrument is being used for its purpose. As one example, a “front side” of an instrument refers to a side that is facing a user of the instrument when the instrument is in use.

As used herein, a “deploy” or “deployment” refers to an act, action, process, system, method, means, or apparatus for inserting an implant or prosthesis into a part, body part, and/or patient. “Deploy” or “deployment” can also refer to an act, action, process, system, method, means, or apparatus for placing something into therapeutic use. A device, system, component, medication, drug, compound, or nutrient may be deployed by a human operator, a mechanical device, an automated system, a computer system or program, a robotic system, or the like.

“Tissue” refers to a structure that makes up a one or more anatomical structures of a patient (i.e., human or animal). Tissue can be soft tissue or hard tissue. “Soft tissue” refers to tissue of a patient (i.e., human or animal). Examples of soft tissue include but are not limited to skin, ligament, tendon, fascia, fat muscle, fibrous tissue, blood vessels, lymph vessels, brain tissue, and/or nerves. “Hard tissue” refers to any human or animal tissue that is not soft tissue. Examples of hard tissue include bone, teeth, tooth enamel, dentin, cementum, cartilage, or the like.

“Topographical” refers to the physical distribution of parts, structures, or features on the surface of, or within, an organ or other anatomical structure, or organism. (Search “define topographical” on google.com. Oxford Languages, Copyright 2022. Oxford University Press. Web., Modified. Accessed 15 Feb. 2022.)

“Boundary” refers to a structure, line, or area where an object, surface, line, area, or operation is or is expected to begin and/or end. A boundary can be similar to a border.

“Landmark registration feature” or “Landmark” refers to a structure configured to engage with a feature, aspect, attribute, or characteristic of a first object to orient and/or position a second object that includes the landmark registration feature with respect to the first object. A variety of structures can serve as a landmark registration feature. For example, a landmark registration feature may include a protrusion, a projection, a tuberosity, a cavity, a void, a divot, a tab, an extension, a hook, a curve, or the like. In the context of bones of a patient a landmark registration feature can include any protuberance, void, divot, concave section, sesamoid, bone spur or other feature on, or extending from, a bone of a patient. A landmark refers to any structure of an anatomical structure that is referenced, contacted, engaged with and/or associated with a landmark registration feature.

“Probe bone engagement surface” refers to a bone engagement surface on one surface of a probe or part of a probe.

“Bone attachment feature” refers to a structure, feature, component, aspect configured to securely connect, couple, attach, and/or engage a structure, component, object, or body with a bone and/or a bone fragment. Examples of a bone attachment feature, include, but are not limited to, a pin, K-wire, screw, or other fastener alone, or in combination with, a hole, passage, and/or opening.

As used herein, “patient-specific osteotomy procedure” refers to an osteotomy procedure that has been adjusted, tailored, modified, or configured to specifically address the needs or desires or a particular patient. In certain aspects, one patient-specific osteotomy procedure may be useable in connection with only one patient. In other aspects, one patient-specific osteotomy procedure may be useable with a number of patients having a particular class of characteristics.

“Ankle fusion procedure” refers to a surgical procedure that seeks to immobilize an ankle joint of a patient. The surgery fuses two or more bones of the ankle of the patient. The surgery involves the use of screws, plates, medical nails, and other hardware or fasteners to achieve bone union. Ankle fusion is considered to be the gold standard for treatment of end-stage ankle arthritis. Ankle fusion trades joint mobility for relief from pain. (Search “ankle fusion” on Wikipedia.com Dec. 21, 2022. CC-BY-SA 3.0 modified. Accessed Jun. 28, 2023.) An ankle fusion procedure may also be referred to as ankle arthrodesis, talocrural joint fusion, tibiotalar arthrodesis, and tibiotalocalcaneal arthrodesis. An ankle fusion procedure can be performed using a variety of approaches to the ankle including an anterior approach, a posterior approach, a lateral approach and a medial approach. Each approach may use common or different instrumentation or implants for the procedure.

“Deformity” refers to any abnormality in or of an organism, a part of an organism, or an anatomical structure of a patient that appears or functions differently than is considered normal, or is common, in relation to the same organism, a part of an organism, or an anatomical structure of other subjects of the same species as the patient. (Search “deformity” on Wikipedia.com Jun. 13, 2023. CC-BY-SA 3.0 Modified. Accessed Jun. 28, 2023.)

“Prescription” or “Prescribed” refers to an instruction, request, direction, determination, designation, authorization, and/or order, as by a physician or nurse practitioner, for the administration of a medicine, preparation of an implant, preparation of an instrument, or other intervention. Often a prescription is written. Prescription can also refer to the prescribed medicine or intervention. (Search “prescription” on wordhippo.com. WordHippo, 2023. Web. Accessed 3 May 2023. Modified.)

“User directions” refers to any request, instruction, direction, input, feedback, prescription, designation, order, directive, or the like from a user of an apparatus, system, device, component, subsystem, or other object. User directions can be created, sent, and/or received in a variety of forms and/or formats, including, but not limited to, a user action in a user interface, a prescription, a form, a conversation, an electronic mail message, a text message, a gesture by the user, or the like. In the context of an osteotomy procedure, user directions can include a set of default settings or choices or instructions for fabrication of a patient-specific instrument or set of instruments, an online form completed by a user (e.g., surgeon), a set of modifications to an original set of user directions, and the like.

“Position” refers to a place or location. (Search “position” on wordhippo.com. WordHippo, 2022. Web. Modified. Accessed 9 Aug. 2022.) Often, a position refers to a place or location of a first object in relation to a place or location of another object. One object can be positioned on, in, or relative to a second object. In addition, a position can refer to a place or location of a first object in relation to a place or location of another object in a virtual environment. For example, a model of one object can be positioned relative to a model of another object in a virtual environment such as a modeling software program.

“Contour” refers to an outline representing or bounding a shape or form of an object. Contour can also refer to an outside limit of an object, area, or surface of the object. (Search “contour” on wordhippo.com. WordHippo, 2023. Web. Modified. Accessed 13 Jun. 2023.)

As used herein, a “stop” refers to an apparatus, instrument, structure, member, device, component, system, or assembly structured, organized, configured, designed, arranged, or engineered to prevent, limit, impede, stop, or restrict motion or movement and/or operation of the another object, member, structure, component, part, apparatus, system, or assembly. In one embodiment, a stop may be used to manage and/or control a cutting tool.

As used herein, a “fastener”, “fixation device”, “fixation hardware” or “fastener system” refers to any structure configured, designed, or engineered to join two structures. Fasteners may be made of a variety of materials including metal, plastic, composite materials, metal alloys, plastic composites, and the like. Examples of fasteners include, but are not limited to screws, rivets, bolts, nails, snaps, hook and loop, set screws, bone screws, nuts, posts, pins, thumb screws, and the like. Other examples of fasteners include, but are not limited to wires, Kirschner wires (K-wire), anchors, bone anchors, plates, bone plates, intramedullary nails or rods or pins, implants, sutures, soft sutures, soft anchors, tethers, interbody cages, fusion cages, and the like.

In certain embodiments, the term fastener may refer to a fastener system that includes two or more structures configured to combine to serve as a fastener. An example of a fastener system is a rod or shaft having external threads and an opening or bore within another structure having corresponding internal threads configured to engage the external threads of the rod or shaft.

In certain embodiments, the term fastener may be used with an adjective that identifies an object or structure that the fastener may be particularly configured, designed, or engineered to engage, connect to, join, contact, or couple together with one or more other structures of the same or different types. For example, a “bone fastener” may refer to an apparatus for joining or connecting one or more bones, one or more bone portions, soft tissue and a bone or bone portion, hard tissue and a bone or bone portion, an apparatus and a bone or portion of bone, or the like.

In certain embodiments, a fastener may be a temporary fastener. A temporary fastener is configured to engage and serve a fastening function for a relatively short period of time. Typically, a temporary fastener is configured to be used until another procedure or operation is completed and/or until a particular event. In certain embodiments, a user may remove or disengage a temporary fastener. Alternatively, or in addition, another structure, event, or machine may cause the temporary fastener to become disengaged.

As used herein, a “fixator” refers to an apparatus, instrument, structure, device, component, member, system, assembly, or module structured, organized, configured, designed, arranged, or engineered to connect two bones or bone fragments or a single bone or bone fragment and another fixator to position and retain the bone or bone fragments in a desired position and/or orientation. Examples of fixators include both those for external fixation as well as those for internal fixation and include, but are not limited to pins, wires, Kirschner wires, screws, anchors, bone anchors, plates, bone plates, intramedullary nails or rods or pins, implants, interbody cages, fusion cages, and the like. Fixation refers to the act of deploying or using a fixator to fix two structures together.

As used herein, an “anchor” refers to an apparatus, instrument, structure, member, part, device, component, system, or assembly structured, organized, configured, designed, arranged, or engineered to secure, retain, stop, and/or hold, an object to or at a fixed point, position, or location. Often, an anchor is coupled and/or connected to a flexible member such as a tether, chain, rope, wire, thread, suture, suture tape, or other like object. Alternatively, or in addition, an anchor may also be coupled, connected, and/or joined to a rigid object or structure. In certain embodiments, an anchor can be a fixation device. Said another way, a fixation device can function as an anchor. In certain embodiments, the term anchor may be used as an adjective that describes a function, feature, or purpose for the noun the adjective ‘anchor’ describes. For example, an anchor hole is a hole that serves as, or can be used as, an anchor.

“Connector” refers to any structure configured, engineered, designed, adapted, and/or arranged to connect one structure, component, element, or apparatus to another structure, component, element, or apparatus. A connector can be rigid, pliable, elastic, flexible, and/or semiflexible. Examples of a connector include but are not limited to any fastener.

“Clearance” refers to a space or opening that provides an unobstructed area to permit one object to move freely in relation to another object.

“Correction,” in a medical context, refers to a process, procedure, device, instrument, apparatus, system, implant, or the like that is configured, designed, developed, fabricated, configured, and/or organized to adjust, translate, move, orient, rotate, or otherwise change an anatomical structure from an original position, location, and/or orientation to a new position, location, and/or orientation that provides a benefit to a patient. The benefit may be one of appearance, anatomical function, pain relief, increased mobility, increased strength, and the like.

“Uniplanar correction” refers to a medical correction, which can include an osteo correction, in one plane (e.g., one of a sagittal plane, a transverse plane, and a coronal/frontal plane) of an anatomical structure such as a foot, hand, or body of a patient.

“Biplanar correction” refers to a medical correction, which can include an osteo correction, in two planes (e.g., two of a sagittal plane, a transverse plane, and a coronal/frontal plane) of an anatomical structure such as a foot, hand, or body of a patient.

“Triplane correction” refers to a medical correction, which can include an osteo correction, in three planes (e.g., all three planes of a sagittal plane, a transverse plane, and a coronal/frontal plane) of an anatomical structure such as a foot, hand, or body of a patient.

“Probe” refers to a medical instrument used to explore, identify, locate, or register to, wounds, organs, and/or anatomical structures including a joint or an articular surface. In certain embodiments, a probe can be thin and/or pointed. In one embodiment, a probe is connected, integrated with, and/or coupled to another structure or instrument. In such an embodiment, the probe may serve to facilitate proper positioning of the another structure or instrument. For example, the probe may be used to identify and/or locate a particular anatomical structure and the positioning of the probe may then cause the connected structure or instrument to also be positioned in a desired location relative to one or more anatomical structures.

“Fusion” refers to a natural process of bone growth and generation in which two separate bones and/or bone fragments grow together as new bone grows when the two separate bones and/or bone fragments contact each other. Often, fusion is facilitated by compression of the two separate bones and/or bone fragments towards each other.

As used herein, “manufacturing tool” or “fabrication tool” refers to a manufacturing or fabrication process, tool, system, or apparatus which creates an object, device, apparatus, feature, or component using one or more source materials. A manufacturing tool or fabrication tool can use a variety of manufacturing processes, including but not limited to additive manufacturing, subtractive manufacturing, forging, casting, and the like. The manufacturing tool can use a variety of materials including polymers, thermoplastics, metals, biocompatible materials, biodegradable materials, ceramics, biochemicals, and the like. A manufacturing tool may be operated manually by an operator, automatically using a computer numerical controller (CNC), or a combination of these techniques.

“Friction fit” refers to a type of joint or connection that is created between two components by means of friction. A joint or connection that is formed using a friction fit may or may not include the use of additional fasteners such as screws, bolts, or adhesives. In a friction fit, the components are designed or configured to fit tightly together, creating enough friction between the surfaces to hold them securely in place, at least temporarily. The friction force is generated by the compressive force that is experienced between the components, and can be strong enough to prevent the components from separating under normal conditions. (© ChatGPT March 23 Version, Modified, accessed chat.openai.com/chat May 2, 2023).

As used herein, “osteotomy procedure” or “surgical osteotomy” or “osteotomy” refers to a surgical operation in which one or more bones are cut to shorten or lengthen them or to change their alignment. The procedure can include removing one or more portions of bone and/or adding one or more portions of bone or bone substitutes. (Search “osteotomy” on Wikipedia.com Feb. 3, 22, 2021. CC-BY-SA 3.0 modified. Accessed Feb. 15, 2022.) As used herein, “patient-specific osteotomy procedure” refers to an osteotomy procedure that has been adjusted, tailored, modified, or configured to specifically address the anatomy, physiology, condition, abnormalities, needs, or desires of a particular patient. In certain aspects, one patient-specific osteotomy procedure may be useable in connection with only one patient. In other aspects, one patient-specific osteotomy procedure may be useable with a number of patients having a particular class of characteristics. In certain aspects, a patient-specific osteotomy procedure may refer to a non-patient-specific osteotomy procedure that includes one or more patient-specific implants and/or instrumentation. In another aspects, a patient-specific osteotomy procedure may refer to a patient-specific osteotomy procedure that includes one or more patient-specific implants, patient-specific surgical steps, and/or patient-specific instrumentation.

“Wedge osteotomy” refers to an osteotomy procedure in which one or more wedges are used as part of the procedure. Generally, wedge osteotomies can be of one of two types, open wedge and closing wedge. The type of osteotomy refers to how the procedure changes the relation between two parts of a bone involved in the osteotomy. In an open wedge osteotomy a wedge of bone or graft or other material is inserted in between two parts of a bone. Consequently, a wedge shape is “opened” in the bone. In a close wedge osteotomy or closing wedge osteotomy a wedge of bone is removed from a bone. Consequently, a wedge shape formed in the bone is “closed.”

“Metatarsal” is a bone of a foot of a human or animal. In a human, a foot typically includes five metatarsals which are identified by number starting from the most medial metatarsal, which is referred to as a first metatarsal and moving laterally the next metatarsal is the second metatarsal, and the naming continues in like manner for the third, fourth, and fifth metatarsal. The metatarsal bone includes three parts a base which is a part that is at a proximal end of the metatarsal, a head which is a part that is at a distal end of the metatarsal, and a shaft or neck connects the base to the head.

“Epiphyses” refers to the rounded end of a long bone, at long bone's joint with adjacent bone(s). Between the epiphysis and diaphysis (the long midsection of the long bone) lies the metaphysis, including the epiphyseal plate (growth plate). At the joint, the epiphysis is covered with articular cartilage; below that covering is a zone similar to the epiphyseal plate, known as subchondral bone. (Search ‘epiphysis’ on Wikipedia.com 17 Jun. 2022. Modified. Accessed Aug. 1, 2022.) “Metaphysis” refers to the neck portion of a long bone between the epiphysis and the diaphysis. The metaphysis contains the growth plate, the part of the bone that grows during childhood, and as the metaphysis grows the metaphysis ossifies near the diaphysis and the epiphyses. (Search ‘metaphysis’ on Wikipedia.com 17 Jun. 2022. Modified. Accessed Aug. 1, 2022.) “Diaphysis” refers to the main or midsection (shaft) of a long bone. The diaphysis is made up of cortical bone and usually contains bone marrow and adipose tissue (fat). The diaphysis is a middle tubular part composed of compact bone which surrounds a central marrow cavity which contains red or yellow marrow. In diaphysis, primary ossification occurs. (Search ‘diaphysis’ on Wikipedia.com 17 Jun. 2022. Modified. Accessed Aug. 1, 2022.)

“Metaphyseal Diaphyseal Junction” or “MDJ” refers to an area of a long bone between the Metaphysis and the Diaphysis. This area can also include or be referred to as the epiphyseal plate (growth) plate. For certain surgical procedures, performing an osteotomy at or near the metaphyseal diaphyseal junction may be advantageous and desirable to promote rapid fusion of two cut faces formed in the osteotomy and bone growth to close the osteotomy, and/or may mitigate the risk of a nonunion of the osteotomy.

As used herein, a “base” refers to a main or central structure, component, or part of a structure. A base is often a structure, component, or part upon which, or from which other structures extend into, out of, away from, are coupled to, or connect to. A base may have a variety of geometric shapes and configurations. A base may be rigid or pliable. A base may be solid or hollow. A base can have any number of sides. In one embodiment, a base may include a housing, frame, or framework for a larger system, component, structure, or device. In certain embodiments, a base can be a part at the bottom or underneath a structure designed to extend vertically when the structure is in a desired configuration or position. Certain bones such as a metatarsal bone can include a base as one structural component of the bone.

As used herein, “anatomic data” refers to data identified, used, collected, gathered, and/or generated in connection with an anatomy of a human or animal. Examples of anatomic data may include location data for structures, both independent, and those connected to other structures within a coordinate system. Anatomic data may also include data that labels or identifies one or more anatomical structures. Anatomic data can include volumetric data, material composition data, and/or the like. Anatomic data can be generated based on medical imaging data or measurements using a variety of instruments including monitors and/or sensors. Anatomic data can be gathered, measured, or collected from anatomical models and/or can be used to generate, manipulate, or modify anatomical models.

A bone model or anatomic model of a patient's body or body part(s) may be generated by computing devices that analyze medical imaging images. Structures of a patient's body can be determined using a process called segmentation.

“Positioner” or “positioning guide” refers to any structure, apparatus, surface, device, system, feature, or aspect configured to position, move, translate, manipulate, or arrange one object in relation to another. In certain embodiments, a positioner can be used for one step in surgical procedure to position, arrange, orient, and/or reduce one bone or bone fragment relative to another. In such embodiments, the positioner may be referred to as a bone positioner. In certain embodiments, the term positioner or positioning guide may be preceded by an adjective that identifies the structure, implement, component, or instrument that may be used with, positioned by, and/or guided by with the positioner. For example, a “pin positioner” may be configured to accept a pin or wire such as a K-wire and serve to position or place the pin relative to another structure such as a bone.

“Reduction guide” or “reducer” refers to any structure, apparatus, surface, device, system, feature, or aspect configured, designed, engineered, or fabricated to reduce or aide a user in the reduction of one bone or bone fragment or implant in relation to another bone or bone fragment or implant.

“Rotation guide” or “rotator” refers to any structure, apparatus, surface, device, system, feature, or aspect configured, designed, engineered, or fabricated to rotate or aid a user in the rotation of one structure relative to another structure. In certain embodiments, a rotation guide or rotator may be used to help a surgeon rotate one or more bones, parts of bones, bone fragment, an implant, or other anatomical structure, either alone or in relation to another one or more bones, parts of bones, bone fragments, implants, or other anatomical structures.

“Trajectory” refers to a path a body travels or a path configured for a body to travel through space. (Search “trajectory” on wordhippo.com. WordHippo, 2023. Web. Modified. Accessed 13 Jun. 2023.)

“Trajectory guide” or “trajectory indicator” or “targeting guide” refers to any structure, apparatus, surface, device, system, feature, or aspect configured to indicate, identify, guide, place, position, or otherwise assist in marking or deploying a fastener or other structure along a desired trajectory for one or more subsequent steps in a procedure.

“Metatarsal base resection guide” refers to a resection guide designed, engineered, fabricated, or intended for use with, one, in, or about a base part, section, surface, portion, or aspect of a metatarsal for one or more steps of a medical procedure. The metatarsal base resection guide may be used to form an osteotomy, to resect a wedge for a closing wedge procedure, resect a bone wedge that preserves a cortical layer of bone opposite the resected bone wedge, form an osteotomy that uniplanar wedge, a biplanar wedge, or a triplane wedge. Various embodiments of a metatarsal base resection guide may be used on a medial surface, a dorsal surface, a lateral surface, or a plantar surface of a single metatarsal. Alternatively, or in addition, various embodiments of a metatarsal base resection guide can be used on two or more metatarsals.

“Reduction guide” or “reducer” refers to any structure, apparatus, surface, device, system, feature, or aspect configured, designed, engineered, or fabricated to reduce or aide a user in the reduction of one bone or bone fragment or implant in relation to another bone or bone fragment or implant.

“Fastener guide” or “reducer” refers to any structure, apparatus, surface, device, system, feature, or aspect configured, designed, engineered, or fabricated to guide or direct a fastener into a bone as part of deploying the fastener. Examples of a fastener guide include an opening in a structure that is sized and/or oriented for deployment of a fastener such as a bone screw, a reference pin for aligning a fastener for deployment at a desired orientation and/or trajectory, and the like.

As used herein, a “guard” refers to an apparatus, instrument, structure, member, device, component, system, or assembly structured, organized, configured, designed, arranged, or engineered to prevent, limit, impede, stop, or restrict motion, action, or movement and/or operation of the another object, member, structure, component, part, apparatus, system, or assembly beyond a certain parameter such as a boundary. Said another way, a “guard” refers to an apparatus, instrument, structure, member, device, component, system, or assembly structured, organized, configured, designed, arranged, or engineered to retain, maintain, hold, keep, or restrict motion, action, or movement and/or operation of the another object, member, structure, component, part, apparatus, system, or assembly within or at one or more parameters such as a boundary.

As used herein, “artificial intelligence” refers to intelligence demonstrated by machines, unlike the natural intelligence displayed by humans and animals, which involves consciousness and emotionality. The distinction between artificial intelligence and natural intelligence categories is often revealed by the acronym chosen. ‘Strong’ AI is usually labelled as artificial general intelligence (AGI) while attempts to emulate ‘natural’ intelligence have been called artificial biological intelligence (ABI). Leading AI textbooks define the field as the study of “intelligent agents”: any device that perceives its environment and takes actions that maximize its chance of achieving its goals. The term “artificial intelligence” can also be used to describe machines that mimic “cognitive” functions that humans associate with the human mind, such as “learning” and “problem solving”. (Search “artificial intelligence” on Wikipedia.com Jun. 25, 2021. CC-BY-SA 3.0 Modified. Accessed Jun. 25, 2021.)

As used herein, “segmentation” or “image segmentation” refers to the process of partitioning an image into different meaningful segments. These segments may correspond to different tissue classes, organs, pathologies, bones, or other biologically relevant structures. Medical image segmentation accommodates imaging ambiguities such as by low contrast, noise, and other imaging ambiguities.

Certain computer vision techniques can be used or adapted for image segmentation. For example, the techniques and or algorithms for segmentation may include, but are not limited to: Atlas-Based Segmentation: For many applications, a clinical expert can manually label several images; segmenting unseen images is a matter of extrapolating from these manually labeled training images. Methods of this style are typically referred to as atlas-based segmentation methods. Parametric atlas methods typically combine these training images into a single atlas image, while nonparametric atlas methods typically use all of the training images separately. Atlas-based methods usually require the use of image registration in order to align the atlas image or images to a new, unseen image.

Image registration is a process of correctly aligning images; Shape-Based Segmentation: Many methods parametrize a template shape for a given structure, often relying on control points along the boundary. The entire shape is then deformed to match a new image. Two of the most common shape-based techniques are Active Shape Models and Active Appearance Models; Image-Based Segmentation: Some methods initiate a template and refine its shape according to the image data while minimizing integral error measures, like the Active contour model and its variations; Interactive Segmentation: Interactive methods are useful when clinicians can provide some information, such as a seed region or rough outline of the region to segment. An algorithm can then iteratively refine such a segmentation, with or without guidance from the clinician. Manual segmentation, using tools such as a paint brush to explicitly define the tissue class of each pixel, remains the gold standard for many imaging applications. Recently, principles from feedback control theory have been incorporated into segmentation, which give the user much greater flexibility and allow for the automatic correction of errors; Subjective surface Segmentation: This method is based on the idea of evolution of segmentation function which is governed by an advection-diffusion model. To segment an object, a segmentation seed is needed (that is the starting point that determines the approximate position of the object in the image). Consequently, an initial segmentation function is constructed. With the subjective surface method, the position of the seed is the main factor determining the form of this segmentation function; and Hybrid segmentation which is based on combination of methods. (Search “medical image computing” on Wikipedia.com Jun. 24, 2021. CC-BY-SA 3.0 Modified. Accessed Jun. 24, 2021.)

As used herein, “medical imaging” refers to a technique and process of imaging the interior of a body for clinical analysis and medical intervention, as well as visual representation of the function of some organs or tissues (physiology). Medical imaging seeks to reveal internal structures hidden by the skin and bones, as well as to diagnose and treat disease. Medical imaging may be used to establish a database of normal anatomy and physiology to make possible identification of abnormalities. Medical imaging in its widest sense, is part of biological imaging and incorporates radiology, which uses the imaging technologies of X-ray radiography, magnetic resonance imaging, ultrasound, endoscopy, elastography, tactile imaging, thermography, medical photography, nuclear medicine functional imaging techniques as positron emission tomography (PET) and single-photon emission computed tomography (SPECT). Another form of X-ray radiography includes computerized tomography (CT) scans in which a computer controls the position of the X-ray sources and detectors. Magnetic Resonance Imaging (MRI) is another medical imaging technology. Measurement and recording techniques that are not primarily designed to produce images, such as electroencephalography (EEG), magnetoencephalography (MEG), electrocardiography (ECG), and others, represent other technologies that produce data susceptible to representation as a parameter graph vs. time or maps that contain data about the measurement locations. In certain embodiments bone imaging includes devices that scan and gather bone density anatomic data. These technologies may be considered forms of medical imaging in certain disciplines. (Search “medical imaging” on Wikipedia.com Jun. 16, 2021. CC-BY-SA 3.0 Modified. Accessed Jun. 23, 2021.) Data, including images, text, and other data associated with medical imaging is referred to as patient imaging data. As used herein, “patient imaging data” refers to data identified, used, collected, gathered, and/or generated in connection with medical imaging and/or medical imaging data. Patient imaging data can be shared between users, systems, patients, and professionals using a common data format referred to as Digital Imaging and Communications in Medicine (DICOM) data. DICOM data is a standard format for storing, viewing, retrieving, and sharing medical images.

As used herein, “medical image computing” or “medical image processing” or “medical imaging” refers to systems, software, hardware, components, and/or apparatus that involve and combine the fields of computer science, information engineering, electrical engineering, physics, mathematics and medicine. Medical image computing develops computational and mathematical methods for working with medical images and their use for biomedical research and clinical care. One goal for medical image computing is to extract clinically relevant information or knowledge from medical images. While closely related to the field of medical imaging, medical image computing focuses on the computational analysis of the images, not their acquisition. The methods can be grouped into several broad categories: image segmentation, image registration, image-based physiological modeling, and others. (Search “medical image computing” on Wikipedia.com Jun. 24, 2021. CC-BY-SA 3.0 Modified. Accessed Jun. 24, 2021.) Medical image computing may include one or more processors or controllers on one or more computing devices. Such processors or controllers may be referred to herein as medical image processors. Medical imaging and medical image computing together can provide systems and methods to image, quantify and fuse both structural and functional information about a patient in vivo. These two technologies include the transformation of computational models to represent specific subjects/patients, thus paving the way for personalized computational models. Individualization of generic computational models through imaging can be realized in three complementary directions: definition of the subject-specific computational domain (anatomy) and related subdomains (tissue types); definition of boundary and initial conditions from (dynamic and/or functional) imaging; and characterization of structural and functional tissue properties. Medical imaging and medical image computing enable the translation of models to the clinical setting with both diagnostic and therapeutic applications. (Id.) In certain embodiments, medical image computing can be used to generate a bone model, a patient-specific model, and/or a patent specific instrument from medical imaging and/or medical imaging data.

As used herein, “model” refers to an informative representation of an object, person or system. Representational models can be broadly divided into the concrete (e.g. physical form) and the abstract (e.g. behavioral patterns, especially as expressed in mathematical form). In abstract form, certain models may be based on data used in a computer system or software program to represent the model. Such models can be referred to as computer models. Computer models can be used to display the model, modify the model, print the model (either on a 2D medium or using a 3D printer or additive manufacturing technology). Computer models can also be used in environments with models of other objects, people, or systems. Computer models can also be used to generate simulations, display in virtual environment systems, display in augmented reality systems, or the like. Computer models can be used in Computer Aided Design (CAD) and/or Computer Aided Manufacturing (CAM) systems. Certain models may be identified with an adjective that identifies the object, person, or system the model represents. For example, a “bone” model is a model of a bone, and a “heart” model is a model of a heart. (Search “model” on Wikipedia.com Jun. 13, 2021. CC-BY-SA 3.0 Modified. Accessed Jun. 23, 2021.) As used herein, “additive manufacturing” refers to a manufacturing process in which materials are joined together in a process that repeatedly builds one layer on top of another to generate a three-dimensional structure or object. Additive manufacturing may also be referred to using different terms including: additive processes, additive fabrication, additive techniques, additive layer manufacturing, layer manufacturing, freeform fabrication, ASTM F2792 (American Society for Testing and Materials), and 3D printing. Additive manufacturing can build the three-dimensional structure or object using computer-controlled equipment that applies successive layers of the material(s) based on a three-dimensional model that may be defined using Computer Aided Design (CAD) software. Additive manufacturing can use a variety of materials including polymers, thermoplastics, metals, ceramics, biochemicals, and the like. Additive manufacturing may provide unique benefits, as an implant together with the pores and/or lattices can be directly manufactured (without the need to generate molds, tool paths, perform any milling, and/or other manufacturing steps).

“Repository” refers to any data source or dataset that includes data or content. In one embodiment, a repository resides on a computing device. In another embodiment, a repository resides on a remote computing or remote storage device. A repository may comprise a file, a folder, a directory, a set of files, a set of folders, a set of directories, a database, an application, a software application, content of a text, content of an email, content of a calendar entry, and the like. A repository, in one embodiment, comprises unstructured data. A repository, in one embodiment, comprises structured data such as a table, an array, a queue, a look up table, a hash table, a heap, a stack, or the like. A repository may store data in any format including binary, text, encrypted, unencrypted, a proprietary format, or the like.

“Reference” refers to any apparatus, structure, device, system, component, marking, and/or indicator organized, configured, designed, engineered, and/or arranged to serve as a source of information or a point of comparison used to support or establish knowledge, truth, or quality. (© ChatGPT January 9 Version, Modified, accessed chat.openai.com/chat Jan. 28, 2023). In certain embodiments, a reference can serve as a starting point or initial position for one or more steps in a surgical procedure. A reference may be a type of fiducial. In certain embodiments, “reference” can be with an adjective describing the reference. For example, a “model reference” is a reference within a model such as a computer model. A model reference refers to any feature, aspect, and/or component within a model. Examples of a model reference include, but are not limited to, a point, a plane, a line, a plurality of points, a surface, an anatomical structure, a shape, or the like. An “anatomical reference” is a reference within, on, near, or otherwise associated with an anatomical structure such as a bone. A reference (e.g., model, actual, virtual, and/or real) may also be referred to as a reference feature.

“Reference feature” refers to a feature configured for use as a point, plane, axis, or line of reference (aka a reference). A reference or reference feature can be used to position, measure, orient, fixation, couple, engage, and/or align one object or structure with another object or structure. In certain embodiments, a reference or reference feature can serve as a baseline, a ground truth, a waypoint, a control point, a landmark, and/or the like. A reference feature can facilitate moving from one coordinate system or frame of reference in a virtual environment to a position, location, frame of reference, environment, or orientation on, or in, an actual object, structure, device, apparatus, anatomical structure, or the like. Advantageously, a reference feature can coordinate objects, models, or structures in a digital or virtual model or representation with corresponding objects or structures (e.g., anatomical structures) of actual physical objects or structures. Said another way, a reference feature can serve to map from a virtual or modeled object to an actual or physical object.

As used herein, “feature” refers to a distinctive attribute or aspect of something. (Search “feature” on google.com. Oxford Languages, 2021. Web. 20 Apr. 2021.) A feature may include one or more apparatuses, structures, objects, systems, sub-systems, devices, or the like. A feature may include a modifier that identifies a particular function or operation and/or a particular structure relating to the feature. Examples of such modifiers applied to a feature, include, but are not limited to, “attachment feature,” “alignment feature,” “securing feature,” “placement feature,” “protruding feature,” “engagement feature,” “disengagement feature,” “resection feature”, “guide feature”, “alignment feature,” and the like.

As used herein, a “marking” or “marker” refers to a symbol, letter, lettering, word, phrase, icon, design, color, diagram, indicator, figure, structure, device, apparatus, surface, component, system, or combination of these designed, intended, structured, organized, configured, programmed, arranged, or engineered to communication information and/or a message to a user receiving, viewing, or encountering the marking. The marking or “marker” can include one or more of a tactile signal, a visual signal or indication, an audible signal, and the like. In one embodiment, a marking may comprise a number or set letters, symbols, or words positioned on a surface, structure, color, color scheme, or device to convey a desired message or set of information.

“Set” refers to a collection of objects. A set can have zero or more objects in the collection. Generally, a set includes one or more objects in the collection.

As used herein, a “sleeve” refers to structure that is narrow and longer longitudinally than the structure is wide. In certain embodiments, a sleeve serves to surround, enclose, wrap, and/or contain something else. In certain embodiments, a sleeve may surround, enclose, wrap, and/or contain a passage or void. (Search “sleeve” on wordhippo.com. WordHippo, 2021. Web. Accessed 15 Nov. 2021. Modified.) In certain embodiments, the term sleeve may be preceded by an adjective that identifies the structure, implement, component or instrument that may be used with, inserted into or associated with the sleeve. For example, a “pin sleeve” may be configured to accept a pin or wire such as a K-wire, a “drive sleeve” may be configured to accept a drill or drill bit, a “fixation member sleeve” may be configured to accept a fastener or fixation member.

As used herein, a “fixation” or “fixation device” refers to an apparatus, instrument, structure, device, component, member, system, assembly, step, process, or module structured, organized, configured, designed, arranged, or engineered to connect two structures either permanently or temporarily. The two structures may be one or the other or both of manmade and/or biological tissues, hard tissues such as bones, teeth or the like, soft tissues such as ligament, cartilage, tendon, or the like. In certain embodiments, fixation is used as an adjective to describe a device or component or step in securing two structures such that the structures remain connected to each other in a desired position and/or orientation. Fixation devices can also serve to maintain a desired level of tension, compression, or redistribute load and stresses experienced by the two structures and can serve to reduce relative motion of one part relative to others. Examples of fixation devices are many and include both those for external fixation as well as those for internal fixation and include, but are not limited to pins, wires, Kirschner wires (K-wires), screws, anchors, bone anchors, plates, bone plates, intramedullary nails or rods or pins, implants, interbody cages, fusion cages, and the like.

“Fusion” refers to a natural process of bone growth and generation in which two separate bones and/or bone fragments grow together as new bone grows when the two separate bones and/or bone fragments contact each other. Often, fusion is facilitated by compression of the two separate bones and/or bone fragments towards each other.

As used herein, “image registration” refers to a method, process, module, component, apparatus, and/or system that seeks to achieve precision in the alignment of two images. As used here, “image” may refer to either or both an image of a structure or object and another image or a model (e.g., a computer based model or a physical model, in either two dimensions or three dimensions). In the simplest case of image registration, two images are aligned. One image may serve as the target image and the other as a source image; the source image is transformed, positioned, realigned, and/or modified to match the target image. An optimization procedure may be applied that updates the transformation of the source image based on a similarity value that evaluates the current quality of the alignment. An iterative procedure of optimization may be repeated until a (local) optimum is found. An example is the registration of CT and PET images to combine structural and metabolic information. Image registration can be used in a variety of medical applications: Studying temporal changes; Longitudinal studies may acquire images over several months or years to study long-term processes, such as disease progression. Time series correspond to images acquired within the same session (seconds or minutes). Time series images can be used to study cognitive processes, heart deformations and respiration; Combining complementary information from different imaging modalities. One example may be the fusion of anatomical and functional information.

Since the size and shape of structures vary across modalities, evaluating the alignment quality can be more challenging. Thus, similarity measures such as mutual information may be used; Characterizing a population of subjects. In contrast to intra-subject registration, a one-to-one mapping may not exist between subjects, depending on the structural variability of the organ of interest. Inter-subject registration may be used for atlas construction in computational anatomy. Here, the objective may be to statistically model the anatomy of organs across subjects; Computer-assisted surgery: in computer-assisted surgery pre-operative images such as CT or MRI may be registered to intra-operative images or tracking systems to facilitate image guidance or navigation. There may be several considerations made when performing image registration: The transformation model. Common choices are rigid, affine, and deformable transformation models. B-spline and thin plate spline models are commonly used for parameterized transformation fields. Non-parametric or dense deformation fields carry a displacement vector at every grid location; this may use additional regularization constraints. A specific class of deformation fields are diffeomorphisms, which are invertible transformations with a smooth inverse; The similarity metric. A distance or similarity function is used to quantify the registration quality. This similarity can be calculated either on the original images or on features extracted from the images. Common similarity measures are sum of squared distances (SSD), correlation coefficient, and mutual information. The choice of similarity measure depends on whether the images are from the same modality; the acquisition noise can also play a role in this decision. For example, SSD may be the optimal similarity measure for images of the same modality with Gaussian noise. However, the image statistics in ultrasound may be significantly different from Gaussian noise, leading to the introduction of ultrasound specific similarity measures.

Multi-modal registration may use a more sophisticated similarity measure; alternatively, a different image representation can be used, such as structural representations or registering adjacent anatomy; The optimization procedure. Either continuous or discrete optimization is performed. For continuous optimization, gradient-based optimization techniques are applied to improve the convergence speed. (Search “medical image computing” on Wikipedia.com Jun. 24, 2021. CC-BY-SA 3.0 Modified. Accessed Jun. 25, 2021.)

“Register” or “Registration” refers to an act of aligning, mating, contacting, engaging, or coupling one or more parts and/or surfaces of one object in relation to one or more parts and/or surfaces of another object. Registering and/or registration can include two parts or surfaces of different object abutting each other and/or coming into close proximity to each other. Often, the one or more parts and/or surfaces of one object include protrusions and/or depressions that are the inverse or mirror configuration of protrusions and/or depressions of one or more parts and/or surfaces of the other object.

“Registration key” refers to a structure, surface, feature, module, component, apparatus, and/or system that facilitates, enables, guides, promotes, precision in the alignment of two objects by way of registration. In one aspect a registration key can include a surface and one or more recesses and/or features of that surface that are configured to fit within corresponding recesses, projections, and/or other features of another structure such as another surface. In one aspect a registration key can include a surface and one or more projections and/or features of, extending from, or connected to that surface that are configured to fit within corresponding recesses, projections, and/or other features of another structure such as another surface. In certain aspects, the features of the registration key may be configured to fit within, or in contact, or in close contact with those of the another structure. In one embodiment, when the two structures align the registration key has served its purpose.

“Shelf” refers to a narrow horizontal surface or structure projecting from a side, surface, structure, wall, cliff, or other surface of a structure. (Search “shelf” on wordhippo.com. WordHippo, 2023. Web. Accessed 16 Aug. 2023. Modified.)

“Section” refers to a separate part, portion, or area of a structure, object, device, apparatus, or container. (Search “section” on wordhippo.com. WordHippo, 2023. Web. Accessed 16 Aug. 2023. Modified.)

“Leg” refers to a part or portion of another structure. Often, a leg can be a narrow, elongated structure resembling a leg of a human.

As used herein, a “resection” refers to a method, procedure, or step that removes tissue from another anatomical structure or body. A resection can include an osteotomy that cuts through a bone or other tissue because the osteotomy still removes at least a minimal amount of tissue. A resection is typically performed by a surgeon on a part of a body of a patient. A resection is a type of osteotomy. (Search “surgery” on Wikipedia.com May 26, 2021. CC-BY-SA 3.0 Modified. Accessed May 26, 2021.) Resection may be used as a noun or a verb. In the verb form, the term is “resect” and refers to an act of performing, or doing, a resection. Past tense of the verb resect is resected.

“Anatomical structure” or “Anatomy” refers to any part or portion of a part of a body of a person, animal, or other patient. Examples of anatomical structures, include but are not limited to, a bone, bones, soft tissue, a joint, joints, a tissue surface, a protrusion, a recess, an opening, skin, hard tissue, teeth, mouth, eyes, hair, nails, fingers, toes, legs, arms, torso, vertebrae, ligaments, tendons, organs, or the like.

“Anatomical reference” refers to any reference(s) that is, or is on, or is in, or is otherwise associated, with an anatomical structure. Examples of anatomical structures, include but are not limited to, a bone, bones, soft tissue, a joint, joints, skin, hard tissue, teeth, mouth, eyes, hair, nails, fingers, toes, legs, arms, torso, vertebrae, ligaments, tendons, organs, a hole, a post, a plurality of holes, a plurality of posts, a fastener, a suture, a clamp, an instrument, an implant, or the like.

As used herein, a “condition” refers to a state of something with regard to its appearance, quality, or working order. In certain aspects, a condition may refer to a patient's state of health or physical fitness or the state of health or physical fitness of an organ or anatomical part of a patient. In certain embodiments, a condition may refer to an illness, pain, discomfort, defect, disease, or deformity of a patient or of an organ or anatomical part of a patient. (Search “condition” on wordhippo.com. WordHippo, 2021. Web. Accessed 8 Dec. 2021. Modified.)

“Bone condition” refers to any of a variety of conditions of bones of a patient. Generally, a bone condition refers to an orientation, position, and/or alignment of one or more bones of the patient relative to other anatomical structures of the body of the patient. Bone conditions may be caused by or result from deformities, misalignment, malrotation, fractures, joint failure, and/or the like. A bone condition includes, but is not limited to, any angular deformities of one or more bone segments in either the lower or upper extremities (for example, tibial deformities, calcaneal deformities, femoral deformities, and radial deformities). Alternatively, or in addition, “bone condition” can refer to the structural makeup and configuration of one or more bones of a patient. Thus bone condition may refer to a state or condition of regions, a thickness of a cortex, bone density, a thickness and/or porosity of internal regions (e.g. whether it is calcaneus or solid) of the bone or parts of the bone such as a head, a base, a shaft, a protuberance, a process, a lamina, a foramen, and the like of a bone, along the metaphyseal region, epiphysis region, and/or a diaphyseal region. “Malrotation” refers to a condition in which a part, typically a part of a patient's body has rotated from a normal position to an unnormal or uncommon position.

As used herein, a “guide” refers to a part, component, member, or structure designed, adapted, configured, or engineered to guide or direct one or more other parts, components, or structures. A guide may be part of, integrated with, connected to, attachable to, or coupled to, another structure, device, or instrument. In one embodiment, a guide may include a modifier that identifies a particular function, location, orientation, operation, type, and/or a particular structure of the guide. Examples of such modifiers applied to a guide, include, but are not limited to, “pin guide” that guides or directs one or more pins, a “cutting guide” that guides or directs the making or one or more cuts, a placement, deployment, or insertion guide that guides or directs the placement, positioning, orientation, deployment, installation, or insertion of a fastener and/or implant, a “cross fixation guide” that guides deployment of a fastener or fixation member, an “alignment guide” that guides the alignment of two or more objects or structures, a “navigation guide” that guides a user in navigating a course or process or procedure such as a surgical procedure, a “resection guide” that serves to guide resection of soft or hard tissue, such as in an osteotomy, a “reduction guide” can serve to guide reduction of one or more bone segments or fragments, an “placement guide” that serves to identify how an object can be placed in relation to another object or structure, and the like. Furthermore, guides may include modifiers applied due to the procedure or location within a patient for which the guide is to be used. For example, where a guide is used at a joint, the guide may be referred to herein as an “arthrodesis guide.”

Those of skill in the art will appreciate that a resection feature may take a variety of forms and may include a single feature or one or more features that together form the resection feature. In certain embodiments, the resection feature may take the form of one or more slots or cut channels. Alternatively, or in addition, a resection feature may be referenced using other names including, but not limited to, channel, cut channels, and the like.

“Cross section” or “cross-section” refers to the non-empty intersection of a body in three-dimensional space with a plane, or the analog in higher-dimensional spaces. (Search “cross section” on Wikipedia.com Mar. 7, 2022. Modified. Accessed Sep. 21, 2022.)

As used herein, “intersection” refers to a point, plane, line, or area where two or more other points, lines, planes, or areas each occupy the same space.

“Intersection angle” refers to an angle at which one or more planes, lines, or areas intersect. An intersection angle may be expressed in units of degrees or radians.

“Wedge angle” refers to an angle at which two planes, surfaces, or sides of a wedge shape meet each other. A wedge angle may be expressed in units of degrees or radians.

“Turn” refers to a bend or curve or change in trajectory and/or direction of a road, path, river, slot, channel, or the like. (Search “turn” on wordhippo.com. WordHippo, 2023. Web. Accessed 16 Aug. 2023. Modified.)

“Cut channel” refers to a channel, slot, hole, or opening, configured to facilitate making a cut. In certain embodiments, a cut channel is one example of a resection feature, resection member, and/or resection guide. “Rotation slot” refers to a channel, slot, hole, or opening, configured to facilitate rotating one structure in relation to another structure.

As used herein, “slot” refers to a narrow opening or groove. (search “slot” on Merriam-Webster.com. Merriam-Webster, 2021. Web. 4 Aug. 2021. Modified.)

“Vertex” refers to a point at which lines, structures, trajectories, or pathways intersect. (Search “vertex” on wordhippo.com. WordHippo, 2023. Web. Modified. Accessed 13 Jun. 2023.)

“Hole” refers to a gap, an opening, an aperture, a port, a portal, a space or recess in a structure, a void in a structure, or the like. In certain embodiments, a hole can refer to a structure configured specifically for receiving something and/or for allowing access. In certain embodiments, a hole can pass through a structure. In other embodiments, an opening can exist within a structure but not pass through the structure. A hole can be two-dimensional or three-dimensional and can have a variety of geometric shapes and/or cross-sectional shapes, including, but not limited to a rectangle, a square, or other polygon, as well as a circle, an ellipse, an ovoid, or other circular or semi-circular shape. As used herein, the term “hole” can include one or more modifiers that define specific types of “holes” based on the purpose, function, operation, position, or location of the “hole.” As one example, a “fastener hole” refers to an “hole” adapted, configured, designed, or engineered to accept or accommodate a “fastener.”

As used herein, an “opening” refers to a gap, a hole, an aperture, a port, a portal, a slit, a space or recess in a structure, a void in a structure, or the like. In certain embodiments, an opening can refer to a structure configured specifically for receiving something and/or for allowing access. In certain embodiments, an opening can pass through a structure. In such embodiments, the opening can be referred to as a window. In other embodiments, an opening can exist within a structure but not pass through the structure. In other embodiments, an opening can initiate on a surface or at an edge or at a side of a structure and extend into the structure for a distance, but not pass through or extend to another side or edge of the structure. In other embodiments, an opening can initiate on a surface or at an edge or at a side of a structure and extend into the structure until the opening extends through or extends to another side or edge of the structure. An opening can be two-dimensional or three-dimensional and can have a variety of geometric shapes and/or cross-sectional shapes, including, but not limited to a rectangle, a square, or other polygon, as well as a circle, an ellipse, an ovoid, or other circular or semi-circular shape. As used herein, the term “opening” can include one or more modifiers that define specific types of “openings” based on the purpose, function, operation, position, or location of the “opening.” As one example, a “fastener opening” refers to an “opening” adapted, configured, designed, or engineered to accept or accommodate a “fastener.”

As used herein, an “interface,” “user interface,” or “engagement interface” refers to an area, a boundary, or a place at which two separate and/or independent structures, members, apparatus, assemblies, components, and/or systems join, connect, are coupled, or meet and act on, or communicate, mechanically and/or electronically, with each other. In certain embodiments, “interface” may refer to a surface forming a common boundary of two bodies, spaces, structures, members, apparatus, assemblies, components, or phases. (search “interface” on Merriam-Webster.com. Merriam-Webster, 2021. Web. 15 Nov. 2021. Modified.) In certain embodiments, the term interface may be used with an adjective that identifies a type or function for the interface. For example, an engagement or coupling interface may refer to one or more structures that interact, connect, or couple to mechanically join or connect two separate structures, each connected to a side of the interface. In another example, a user interface may refer to one or more mechanical, electrical, or electromechanical structures that interact with or enable a user to provide user input, instructions, input signals, data, or data values and receive output, output data, or feedback.

“Resection interface” refers to an interface between a resected portion of tissue and another object, structure, or thing. Often a resection interface is an interface or boundary between one resected portion of an anatomical structure and another resected portion of another anatomical structure. The two anatomical structures can be portions, parts, or fragments of one anatomical structure or two different anatomical structures. A resection interface can be embodied in a variety of shapes and/or configurations, including a point, a line, a plane, a contour, a boundary, or the like. In one embodiment, a resection interface is an interface between two or more cut planes or two or more cut surfaces or two or more cut faces.

“Cortical bone” refers to a type of bone tissue. Cortical bone is a type of bone tissue typically found between an external surface of a bone and an interior area of the bone. Cortical bone is more dense and typically stronger structurally than other types of bone tissue. “Cortical surface” refers to a surface of cortical bone.

“Cortex” refers to an area of bone that extends from an external surface of the bone towards a center part of the bone. The cortex is typically comprised of cortical bone. A cortex can comprise a top natural surface of a bone and extend into the bone a certain depth.

“Transosseous placement feature” refers to a placement feature that extends through one or more bones and that enables, or facilitates, placement of another device, apparatus, or instrument.

“Patient specific feature” refers to a feature, function, structure, device, guide, tool, instrument, apparatus, member, component, system, assembly, module, or subsystem that is adjusted, tailored, modified, organized, configured, designed, arranged, engineered, and/or fabricated to specifically address the anatomy, physiology, condition, abnormalities, needs, or desires of a particular patient or surgeon serving the particular patient. In one aspect, a patient specific feature is unique to a single patient and may include features unique to the patient such as a number of cut channels, a number of bone attachment features, a number of bone engagement surfaces, a number of resection features, a depth of one or more cutting channels, an angle for one or more resection channels, a surface contour, component position, component orientation, and/or other features. “Medial resection guide” refers to a resection guide designed, engineered, fabricated, or intended for use with, one, in, or about a medial part, section, surface, portion, or aspect of an anatomical structure such as a bone, digit, limb, or other anatomical structure for one or more steps of a resection procedure. “Lateral resection guide” refers to a resection guide designed, engineered, fabricated, or intended for use with, one, in, or about a lateral part, section, surface, portion, or aspect of an anatomical structure such as a bone, digit, limb, or other anatomical structure for one or more steps of a resection procedure.

“Prescription” or “Prescribed” refers to a written order, as by a physician or nurse practitioner, for the administration of a medicine, preparation of an implant, preparation of an instrument, or other intervention. Prescription can also refer to the prescribed medicine or intervention. (Search “prescription” on wordhippo.com. WordHippo, 2023. Web. Accessed 3 May 2023. Modified.)

As used herein, “end” refers to a part or structure of an area or span that lies at the boundary or edge. An end can also refer to a point that marks the extent of something and/or a point where something ceases to exist. An end can also refer to an extreme or last part lengthwise of a structure or surface. (search “end” on Merriam-Webster.com. Merriam-Webster, 2021. Web. 4 Aug. 2021. Modified.)

As used herein, “edge” refers to a structure, boundary, or line where an object, surface, or area begins or ends. An edge can also refer to a boundary or perimeter between two structures, objects, or surfaces. An edge can also refer to a narrow part adjacent to a border. (search “edge” on Merriam-Webster.com. Merriam-Webster, 2021. Web. 3 Aug. 2021. Modified.) In certain embodiments, an edge can be a one dimensional or a two-dimensional structure that joins two adjacent structures or surfaces. Furthermore, an edge may be at a perimeter of an object or within a perimeter or boundary of an object.

“Bone fragment” refers to a part of a bone that is normally part of another bone of a patient. A bone fragment may be separate from another bone of a patient due to a deformity or trauma. In one aspect, the bone the bone fragment is normally connected or joined with is referred to as a parent bone.

“Joint” or “Articulation” refers to the connection made between bones in a human or animal body which link the skeletal system to form a functional whole. Joints may be biomechanically classified as a simple joint, a compound joint, or a complex joint. Joints may be classified anatomically into groups such as joints of hand, elbow joints, wrist joints, axillary joints, sternoclavicular joints, vertebral articulations, temporomandibular joints, sacroiliac joints, hip joints, knee joints, ankle joints, articulations of foot, and the like. (Search “joint” on Wikipedia.com Dec. 19, 2021. CC-BY-SA 3.0 Modified. Accessed Jan. 20, 2022.)

“Articular surface” refers to a surface of a bone that contacts, slides against, and/or is part of a joint and/or joint articulation. Each bone included in a joint may have one or more articular surfaces.

“Tarso-metatarsal joint” or “TMT joint” refers to a joint of a patient between a metatarsal bone and one or more cuneiform/tarsal/cuboid bones. The TMT joint may also be referred to as a “Lis Franc” or “Lisfranc” joint after a French surgeon Lisfranc.

“Cut surface” refers to a surface of an object that is created or formed by the removal of one or more parts of the object that includes the original surface. Cut surfaces can be created using a variety of methods, tools, or apparatuses and may be formed using a variety of removal actions, including, but not limited to, fenestrating, drilling, abrading, cutting, sawing, chiseling, digging, scrapping, and the like. Tools and/or methods used for forming a cut surface can include manual, mechanical, motorized, hydraulic, automated, robotic, and the like. In certain embodiments, the cut surface(s) are planar.

“Orientation” refers to a direction, angle, position, condition, state, or configuration of a first object, component, part, apparatus, system, or assembly relative to another object, component, part, apparatus, system, assembly, reference point, reference axis, or reference plane.

“Longitudinal axis” or “Long axis” refers to an axis of a structure, device, object, apparatus, or part thereof that extends from one end of a longest dimension to an opposite end. Typically, a longitudinal axis passes through a center of the structure, device, object, apparatus, or part thereof along the longitudinal axis. The center point used for the longitudinal axis may be a geometric center point and/or a mass center point.

“Mechanical axis” refers to an axis of a long bone such as a femur or tibia. The mechanical axis of a long bone is a straight line connecting the joint center points of the proximal and distal joint regions, whether in the frontal or sagittal plane. A mechanical axis can be useful in defining how the mechanical (weight, gait, flexion, extension, etc.) forces impact the morphology of the bone structure. A mechanical axis and anatomical axis can both help in the surgical planning in relation to deformed bones. (Search “axes of the long bones” on appropedia.com; Amit Dinanath Maurya, OpenSurgiSim (2021-2023). “Axes of the long bones—Mechanical and Anatomical”. SELF. Modified. Accessed Jun. 28, 2023.)

As used herein, a “drive”, “drive feature”, or “drive recess” refers to an apparatus, instrument, structure, member, device, component, system, or assembly structured, organized, configured, designed, arranged, or engineered to receive a torque and transfer that torque to a structure connected or coupled to the drive. At a minimum, a drive is a set of shaped cavities and/or protrusions on a structure that allows torque to be applied to the structure. Often, a drive includes a mating tool, known as a driver. For example, cavities and/or protrusions on a head of a screw are one kind of drive and an example of a corresponding mating tool is a screwdriver, that is used to turn the screw, the drive. Examples of a drive include but are not limited to screw drives such as slotted drives, cruciform drives, square drives, multiple square drives, internal polygon, internal hex drives, penta lobular sockets, hex lobular sockets, combination drives, external drives, tamper-resistant drives, and the like. (Search ‘list of screw drives’ on Wikipedia.com Mar. 12, 2021. Modified. Accessed Mar. 19, 2021.)

“Thread” or “threads” refers to a helical structure used to convert between rotational and linear movement or force. A thread is a ridge wrapped around a cylinder or cone in the form of a helix, with the ridge wrapped around the cylinder being called a straight thread and the ridge wrapped around the cone called a tapered thread. Straight threads or tapered threads are examples of external threads, also referred to as male threads. Threads that a correspond to male threads are referred to as female threads and are formed within the inside wall of a matching hole, passage, or opening of a nut or substrate or other structure. A thread used with a fastener may be referred to as a screw thread and can be an important feature of a simple machine and also as a threaded fastener. The mechanical advantage of a threaded fastener depends on its lead, which is the linear distance the threaded fastener travels in one revolution. (Search ‘screw thread’ on Wikipedia.com Jul. 17, 2022. Modified. Accessed Aug. 1, 2022.)

“Cutting tool” refers to any tool that can be used to cut or resect another object. In particular, a cutting tool can refer to a manual or power tool for cutting or resecting tissue of a patient. Examples of cutting tools include, but are not limited to, a burr, an oscillating saw, a reciprocating saw, a grater saw, a drill, a mill, a side-cutting burr, or the like.

As used herein, a “shaft” refers to a long narrow structure, device, component, member, system, or assembly that is structured, organized, configured, designed, arranged, or engineered to support and/or connect a structure, device, component, member, system, connected to each end of the shaft. Typically, a shaft is configured to provide rigid support and integrity in view of a variety of forces including tensile force, compression force, torsion force, shear force, and the like. In addition, a shaft can be configured to provide rigid structural support and integrity in view of a loads including axial loads, torsional loads, transverse loads, and the like. A shaft may be oriented and function in a variety of orientations including vertical, horizontal, or any orientation between these and in two or three dimensions. A shaft may be made from a variety of materials including, but not limited to, metal, plastic, ceramic, wood, fiberglass, acrylic, carbon, biocompatible materials, biodegradable materials or the like. A shaft may be formed of any biocompatible materials, including but not limited to biocompatible metals such as Titanium, Titanium alloys, stainless steel, carbon fiber, combinations of carbon fiber and a metallic alloy, stainless steel alloys, cobalt-chromium steel alloys, nickel-titanium alloys, shape memory alloys such as Nitinol, biocompatible ceramics, and biocompatible polymers such as Polyether ether ketone (PEEK) or a polylactide polymer (e.g. PLLA) and/or others, or any combination of these materials.

“Head” refers to a device, apparatus, member, component, system, assembly, module, subsystem, circuit, or structure, organized, configured, designed, arranged, or engineered to have a prominent role in a particular feature, function, operation, process, method, and/or procedure for a device, apparatus, member, component, system, assembly, module, subsystem, circuit, or structure the includes, is coupled to, or interfaces with the head. In certain embodiments, the head may sit at the top or in another prominent position when interfacing with and/or coupled to a device, apparatus, member, component, system, assembly, module, subsystem, circuit, or structure.

As used herein, an “interface,” “user interface,” or “engagement interface” refers to an area, a boundary, or a place at which two separate and/or independent structures, members, apparatus, assemblies, components, and/or systems join, connect, are coupled, or meet and act on, or communicate, mechanically and/or electronically, with each other. In certain embodiments, “interface” may refer to a surface forming a common boundary of two bodies, spaces, structures, members, apparatus, assemblies, components, or phases. (search “interface” on Merriam-Webster.com. Merriam-Webster, 2021. Web. 15 Nov. 2021. Modified.) In certain embodiments, the term interface may be used with an adjective that identifies a type or function for the interface. For example, an engagement or coupling interface may refer to one or more structures that interact, connect, or couple to mechanically join or connect two separate structures, each connected to a side of the interface. In another example, a user interface may refer to one or more mechanical, electrical, or electromechanical structures that interact with or enable a user to provide user input, instructions, input signals, data, or data values and receive output, output data, or feedback.

“Cut surface” or “cut face” refers to a surface of an object that is created or formed by the removal of one or more parts of the object that includes the original surface. Cut surfaces or cut faces can be created using a variety of methods, tools, or apparatuses and may be formed using a variety of removal actions, including, but not limited to, fenestrating, drilling, abrading, cutting, sawing, chiseling, digging, scrapping, and the like. Tools and/or methods used for forming a cut surface or cut face can include manual, mechanical, motorized, hydraulic, automated, robotic, and the like.

“Aspect” refers to any specific feature, part, or element of something. (Search “aspect” on wordhippo.com. WordHippo, 2023. Web. Last Accessed 25 Jul. 2024.)

The present disclosure discloses surgical systems and methods by which a bone condition, that can include a deformity, may be corrected or otherwise addressed. Known methods of addressing bone conditions are often limited to a finite range of discretely sized instruments. A patient with an unusual condition, or anatomy that falls between instrument sizes, may not be readily treated with such systems.

Furthermore, patient-specific instruments may be used for various other procedures on the foot, or on other bones of the musculoskeletal system. For example, patient-specific instruments and/or other instruments may be used for various procedures including resection and translation of a head of a long bone, determining where to perform an osteotomy on one or more joints or part of one or more bones, determining ligament or tendon attachment or anchoring points, determining where to form bone tunnels or position anchors, tendon or graft deployment, and the like.

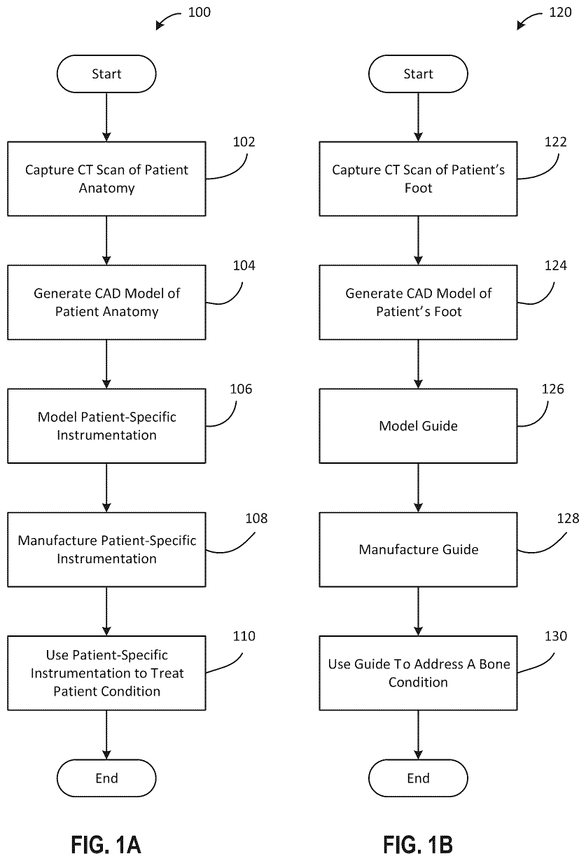

FIG. 1A is a flowchart diagram depicting a method 100 for correcting a bone condition, according to one embodiment. The method 100 may be used for any of a wide variety of bone conditions, including but not limited to deformities, fractures, joint failure, and/or the like. Further, the method 100 may provide correction with a wide variety of treatments, including but not limited to arthroplasty, arthrodesis, fracture repair, and/or the like.

As shown, the method 100 may begin with a step 102 in which a CT scan (or another three-dimensional image, also referred to as medical imaging) of the patient's anatomy is obtained. The step 102 may include capturing a scan of only the particular bone(s) to be treated, or may include capture of additional anatomic information, such as the surrounding tissues. Additionally or alternatively, the step 102 may include receiving a previously captured image, for example, at a design and/or fabrication facility. Performance of the step 102 may result in possession of a three-dimensional model of the patient's anatomy, or three-dimensional surface points that can be used to construct such a three-dimensional model.

After the step 102 has been carried out, the method 100 may proceed to a step 104 in which a CAD model of the patient's anatomy (including one or more bones) is generated. The CAD model may be one example of a bone model. The CAD model may be of any known format, including but not limited to SolidWorks, Catia, AutoCAD, or DXF. In some embodiments, customized software may be used to generate the CAD model from the CT scan. The CAD model may only include the bone(s) to be treated and/or may include surrounding tissues. In alternative embodiments, the step 104 may be omitted, as the CT scan may capture data that can directly be used in future steps without the need for conversion.

In one embodiment, the CAD model generated and/or patient-specific instrumentation, implants, and/or plan for conducting an operative procedure, may be enhanced by the use of advanced computer analysis system, machine learning, and/or automated/artificial intelligence. For example, these technologies may be used to revise a set of steps for a procedure such that a more desirable outcome is achieved.

In a step 106, the CAD model and/or CT scan data may be used to model patient-specific instrumentation that can be used to correct the condition, as it exists in the patient's anatomy. In some embodiments, any known CAD program may be used to view and/or manipulate the CAD model and/or CT scan, and generate one or more instruments that are matched specifically to the size and/or shape of the patient's bone(s). In some embodiments, such instrumentation may include a targeting guide, trajectory guide, drill guide, cutting guide, tendon trajectory guide, capital fragment positioning guide, or similar guide that can be attached to one or more bones, with one or more features that facilitate work on the one or more bones pursuant to a procedure such as arthroplasty or arthrodesis. In some embodiments, performance of the step 106 may include modelling an instrument with a bone engagement surface that is shaped to match the contour of a surface of the bone, such that the bone engagement surface can lie directly on the corresponding contour.

In a step 108, the model(s) may be used to manufacture patient-specific instrumentation and/or implants. This may be done via any known manufacturing method, including casting, forging, milling, additive manufacturing, and/or the like. Additive manufacturing may provide unique benefits, as the model may be directly used to manufacture the instrumentation and/or implants (without the need to generate molds, tool paths, and/or the like beforehand). Such instrumentation may optionally include a targeting guide, trajectory guide, drill guide, cutting guide, positioner, positioning guide, tendon trajectory guide, or the like.

In addition to, or in the alternative to the step 108, the model(s) may be used to select from available sizes of implants and/or instruments or instruments having various attributes and advise the surgeon accordingly. For example, where a range of guides are available for a given procedure, analysis of the CAD data may facilitate pre-operative selection of the optimal guide and/or optimal placement of the guide on the bone. Similarly, if a range of implants and/or instruments may be used for a given procedure, analysis of the CAD data may facilitate pre-operative selection of the optimal implant(s). More particularly, properly-sized spacers, screws, bone plates, and/or other hardware may be pre-operatively selected.

Thus, the result of the step 108 may provision, to the surgeon, of one or more of the following: (1) one or more patient-specific instruments; (2) one or more patient-specific implants; (3) an instrument, selected from one or more available instrument sizes and/or configurations; (4) an implant, selected from one or more available implant sizes and/or configurations; (5) instructions for which instrument(s) to select from available instrument sizes and/or configurations; (6) instructions for which implant(s) to select from available implant sizes and/or configurations; (7) instructions for proper positioning or anchorage of one or more instruments to be used in the procedure; and (8) instructions for proper positioning or anchorage of one or more implants to be used in the procedure. These items may be provided to the surgeon directly, or to a medical device company or representative, for subsequent delivery to the surgeon.

In a step 110, the manufactured instrumentation may be used in surgery to facilitate treatment of the condition. In some embodiments, this may include placing the modelled bone engagement surface against the corresponding contour of the bone used to obtain its shape, and then using the resection feature(s) to guide resection of one or more bones. Then the bone(s) may be further treated, for example, by attaching one or more joint replacement implants (in the case of joint arthroplasty), or by attaching bone segments together (in the case of arthrodesis or fracture repair). Prior to completion of the step 110, the instrumentation may be removed from the patient, and the surgical wound may be closed.

As mentioned previously, the method 100 may be used to correct a wide variety of bone conditions. One example of the method 100 will be shown and described in connection with FIG. 1B, for correction of a bunion deformity of the foot.

In certain embodiments, one or more of a method, apparatus, and/or system of the disclosed solution can be used for training a surgeon to perform a patient-specific procedure or technique. In one embodiment, the CAD model generated and/or patient-specific instrumentation, implants, and/or plan for conducting an operative procedure can be used to train a surgeon to perform a patient-specific procedure or technique.

In one example embodiment, a surgeon may submit a CT scan of a patient's foot to an apparatus or system that implements the disclosed solution. Next, a manual or automated process may be used to generate a CAD model and for making the measurements and correction desired for the patient. In the automated process, an advanced computer analysis system, machine learning and automated/artificial intelligence may be used to generate a CAD model and/or one or more patient-specific instruments and/or operation plans. For example, a patient-specific instrument may be fabricated that is registered to the patient's anatomy using a computer-aided machine (CAM) tool. In addition, a CAM tool may be used to fabricate a 3D structure representative of the patient's anatomy, referred to herein as a patient-specific synthetic cadaver. (e.g. one or more bones of a patient's foot). Next, the patient-specific instrument and the patient-specific synthetic cadaver can be provided to a surgeon who can then rehearse an operation procedure in part or in full before going into an operating room with the patient.

In certain embodiments, the patient-specific instrument or instrument can be used to preposition and/or facilitate pre-drilling holes for a plate system for fixation purposes. Such plate systems may be optimally placed, per a CT scan, after a correction procedure for optimal fixation outcome. In another embodiment, the CAD model and/or automated process such as advanced computer analysis, machine learning and automated/artificial intelligence may be used to measure a depth of the a through a patient-specific resection guide for use with robotics apparatus and/or systems which would control the depth of each cut within the guide to protect vital structures below or adjacent to a bone being cut. In another embodiment, the CAD model and/or automated process such as advanced computer analysis, machine learning and automated/artificial intelligence may be used to define desired fastener (e.g. bone screw) length and/or trajectories through a patient-specific instrument and/or implant. The details for such lengths, trajectories, and components can be detailed in a report provided to the surgeon preparing to perform a procedure.

FIG. 1B is a flowchart diagram depicting a method 120 for correcting or remediating a bone condition, according to one embodiment. The method 120 may be used to prepare for an orthopedic procedure which corrects or remediates a bone, muscle, deformity, and/or tendon condition of a patient.

As shown, the method 120 may begin with a step 122 in which a CT scan (or another three-dimensional image) of the patient's foot is obtained. The step 122 may include capturing a scan of select bones of a patient or may include capturing additional anatomic information, such as the entire foot. Additionally or alternatively, the step 122 may include receipt of previously captured image data. Capture of the entire foot in the step 122 may facilitate proper alignment of the first metatarsal with the rest of the foot (for example, with the second metatarsal). Performance of the step 122 may result in generation of a three-dimensional model of the patient's foot, or three-dimensional surface points that can be used to construct such a three-dimensional model.

After the step 122 has been carried out, the method 120 may proceed to a step 124 in which a CAD model of the relevant portion of the patient's anatomy is generated. The CAD model may optionally include the bones of the entire foot, like the CT scan obtained in the step 122. In alternative embodiments, the step 124 may be omitted in favor of direct utilization of the CT scan data, as described in connection with the step 104.

In a step 126, the CAD model and/or CT scan data may be used to model patient-specific instrumentation that can be used to correct or remediate a bone condition. Such instrumentation may include a guide. In one example, the guide can seat or abut or contact a surface of a bone and including an opening that guides a trajectory for a fastener for a procedure. In some embodiments, performance of the step 126 may include modelling the guide with a bone engagement surface that is shaped to match contours of the surfaces of the bone, such that the bone engagement surface can lie directly on the corresponding contours of the bone.

In a step 128, the model(s) may be used to manufacture patient-specific instrumentation and/or instruments. This may include manufacturing an instrument with the bone engagement surface and/or other features as described above. As in the step 108, the step 128 may additionally or alternatively involve provision of one or more instruments and/or implants from among a plurality of predetermined configurations or sizes. Further, the step 128 may additionally, or alternatively, involve provision of instructions for placement and/or anchorage of one or more instruments and/or instruments to carry out the procedure.

In a step 130, the manufactured instrument may be used in surgery to facilitate treatment of the condition. In certain embodiments, a bone engagement surface of the instrument may be placed against the corresponding contours of the bone. The instrument may include an opening and/or trajectory guide to guide insertion of a trajectory guide such as a temporary fastener such as a K-wire. The instrument may then be removed, and the remaining steps of a surgical procedure performed.

Method 100 and method 120 are merely exemplary. Those of skill in the art will recognize that various steps of the method 100 and the method 120 may be reordered, omitted, and/or supplemented with additional steps not specifically shown or described herein.

As mentioned previously, the method 120 is one species of the method 100; the present disclosure encompasses many different procedures, performed with respect to many different bones and/or joints of the body. Exemplary steps and instrumentation for the method 120 will further be shown and described in connection with the present disclosure. Those of skill in the art will recognize that the method 120 may be used in connection with different instruments; likewise, the instruments of the present disclosure may be used in connection with methods different from the method 100 and the method 120.

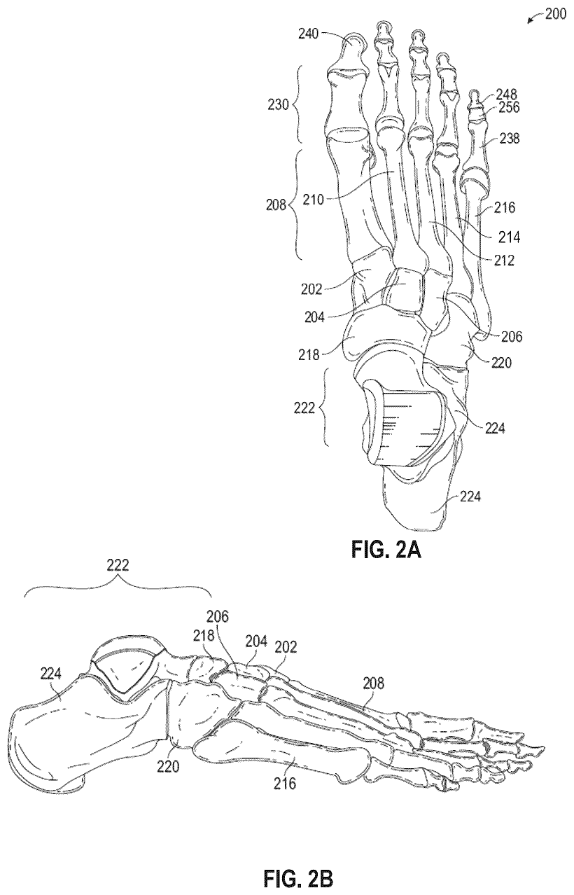

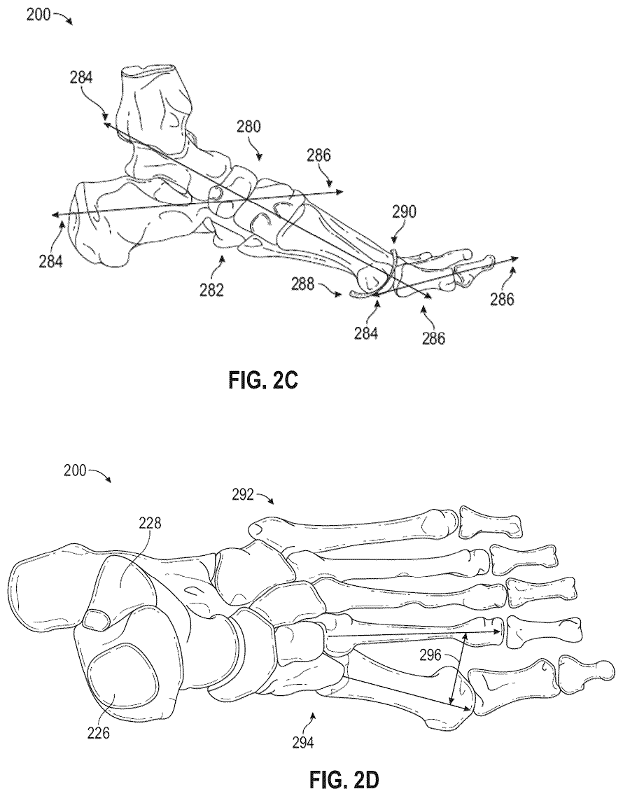

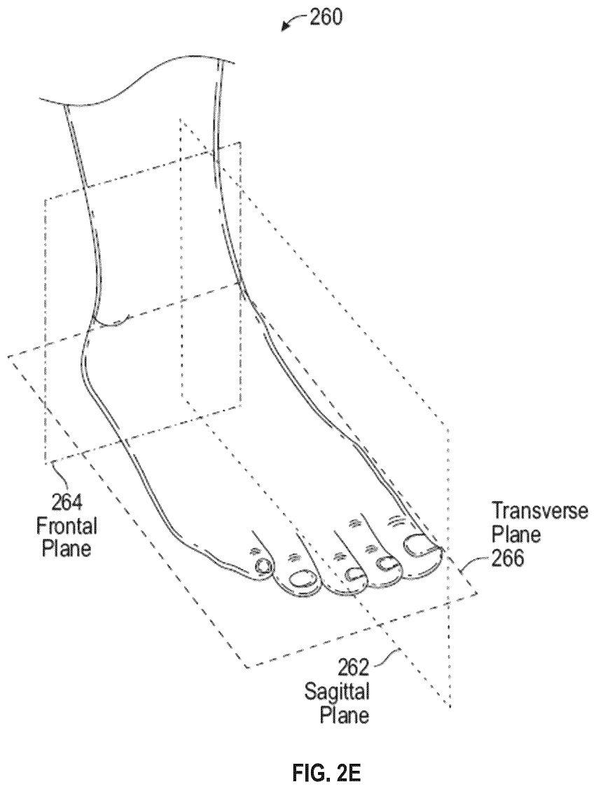

FIG. 2A is a perspective dorsal view of a foot 200. The foot 200 may have a medial cuneiform 202, an intermediate cuneiform 204, lateral cuneiform 206, a first metatarsal 208, a second metatarsal 210, third metatarsal 212, fourth metatarsal 214, fifth metatarsal 216, navicular 218, cuboid 220, talus 222, and calcaneus 224, among others. The medial cuneiform 202 and the intermediate cuneiform 204 may be joined together at a first metatarsocuneiform joint, and the first metatarsal 208 and the second metatarsal 210 may be joined together at a second metatarsocuneiform joint. The foot 200 includes a set of proximal phalanges numbered first through fifth (230, 232, 234, 236, 238) and a set of distal phalanges numbered first through fifth (240, 242, 244, 246, 248) and a set of middle phalanges numbered second through fifth (250, 252, 254, 256).

FIG. 2B is a perspective lateral view of a foot 200, with bones of the foot labeled.

FIG. 2C is a perspective medial view of a foot illustrating a dorsal side 280 and a plantar side 282. The foot 200, as illustrated, may have a tibia 226 and a fibula 228, among others. Dorsal refers to the top of the foot. Plantar refers to the bottom of the foot. Proximal 284 is defined as “closer to the primary attachment point”. Distal 286 is defined as “further away from the attachment point”. Plantarflex or plantarflexion 288 means movement toward the plantar side 282 of a foot or hand, toward the sole or palm. Dorsiflex or dorsiflexion 290 means movement toward the dorsal side 280 of a foot or hand, toward the top. FIG. 2D is a perspective dorsal view of the foot 200. A transverse plane is the plane that shows the top of the foot. A lateral side 292 means a side furthest away from the midline of a body, or away from a plane of bilateral symmetry of the body. A medial side 294 means a side closest to the midline of a body, or toward a plane of bilateral symmetry of the body. For a Lapidus procedure, the intermetatarsal (IM) angle 296 is the angle to be corrected to remove the hallux valgus (bunion) deformity.

FIG. 2E is a view of a foot illustrating common planes 260 of reference for a human foot. FIG. 2E illustrates a sagittal plane 262 that divides the foot into a right section and a left section half. The sagittal plane 262 is perpendicular to frontal or coronal plane 264 and the transverse plane 266. In the foot, the frontal plane 264 generally runs vertically through the ankle and the transverse plane 266 generally runs horizontally through the midfoot and toes of the foot.

Every patient and/or condition is different; accordingly, the degree of angular adjustment needed in each direction may be different for every patient. Use of a patient-specific instrument may help the surgeon obtain an optimal realignment, target, or position a bone tunnel, position one or more resections and/or fasteners and the like. Thus, providing patient-specific instruments, jigs, and/or instrumentation may provide unique benefits.

The present patient-specific instrumentation may be used to correct a wide variety of conditions. Such conditions include, but are not limited to, angular deformities of one bone in either the lower or upper extremities (for example, tibial deformities, calcaneal deformities, femoral deformities, and radial deformities). The present disclosure may also be used to treat an interface between two bones (for example, the ankle joint, metatarsal cuneiform joint, lisfranc's joint, complex Charcot deformity, wrist joint, knee joint, etc.). As one example, an angular deformity or segmental malalignment in the forefoot may be treated, such as is found at the metatarsal cuneiform level, the midfoot level such as the navicular cuneiform junction, hindfoot at the calcaneal cuboid or subtalar joint or at the ankle between the tibia and talar junction. Additionally, patient-specific instruments could be used in the proximal leg between two bone segments or in the upper extremity such as found at the wrist or metacarpal levels.

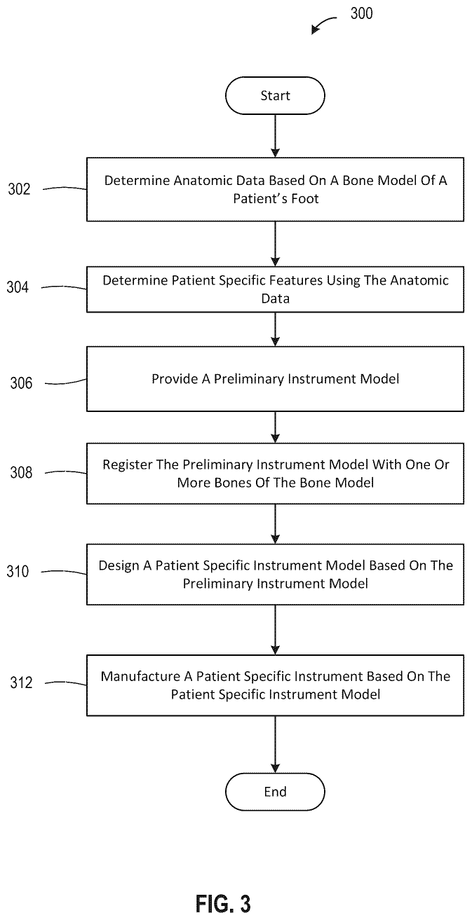

FIG. 3 illustrates a flowchart diagram depicting a method 300 for generating one or more instruments (which may or may not be patient-specific) configured to correct or address a bone or foot condition, according to one embodiment. Prior to steps of the method 300, a bone model (also referred to as CAD model above) is generated. The bone model may be generated using medical imaging of a patient's foot and may also be referred to as an anatomic model. The medical imaging image(s) may be used by computing devices to generate patient imaging data. The patient imaging data may be used to measure and account for orientation of one or more structures of a patient's anatomy. In certain embodiments, the patient imaging data may serve, or be a part of, anatomic data for a patient.

In one embodiment, the method 300 begins after a bone model of a patient's body or body part(s) is generated. In a first step 302, the method 300 may review the bone model and data associated with the bone model to determine anatomic data of a patient's foot.

After step 302, the method 300 may determine 304 one or more angles (e.g., trajectory angle) and/or patient-specific features for a procedure using the anatomic data. “Trajectory angle” refers to a recommended angle for deployment of an instrument, graft, body part, or resection feature angle relative to a bone of a patient for a procedure. In certain embodiments, determining steps, instruments, and/or implants for a corrective procedure may employ an advanced computer analysis system, expert systems, machine learning, and/or automated/artificial intelligence.

Next, the method 300 may proceed and a preliminary instrument model is provided 306 from a repository of template models. A preliminary instrument model is a model of a preliminary instrument.

As used herein, “preliminary instrument” refers to an instrument configured, designed, and/or engineered to serve as a template, prototype, archetype, or starting point for creating, generating, or fabricating a patient-specific instrument. In one aspect, the preliminary instrument may be used, as-is, without any further changes, modifications, or adjustments and thus become a patient-specific instrument. In another aspect, the preliminary instrument may be modified, adjusted, or configured to more specifically address the goals, objectives, or needs of a patient or a surgeon and by way of the modifications become a patient-specific instrument. The patient-specific instrument can be used by a user, such as a surgeon, to guide steps in a surgical procedure, such as an osteotomy, graft harvest (e.g., autograft, allograft, or xenograft), minimally invasive surgical (MIS) procedure, and/or a tendon transfer procedure. Accordingly, a preliminary instrument model can be used to generate a patient-specific instrument. The patient-specific instrument model may be used in a surgical procedure to facilitate one or more steps of the procedure, and may be used to generate a patient-specific instrument that can be used in a surgical procedure for the patient.

In certain embodiments, the preliminary instrument model may be generated based on anatomic data and/or a bone model or a combination of these, and no model or predesigned structure, template, or prototype. Alternatively, or in addition, the preliminary instrument model may be, or may originate from, a template instrument model selected from a set of template instrument models. Each model in the set of template instrument models may be configured to fit an average patient's foot. The template instrument model may subsequently be modified or revised by an automated process or manual process to generate the preliminary instrument model used in this disclosure.

A “bone model” or “anatomic model” refers to a model of a bone of a person. The bone model may model a single bone or a plurality of bones. The modeled bone and/or bones may be positioned in standard anatomical form and/or may be positioned relative to other bones (e.g., models of bones) of a person such that the positions of the bones in the bone model are the same or substantially the same as corresponding bones of a person, such as a patient.

As used herein, “model” refers to an informative representation of an object, person or system. Representational models can be broadly divided into the concrete (e.g. physical form) and the abstract (e.g. behavioral patterns, especially as expressed in mathematical form). In abstract form, certain models may be based on data used in a computer system or software program to represent the model. Such models can be referred to as computer models. Computer models can be used to display the model, modify the model, print the model (either on a 2D medium or using a 3D printer or additive manufacturing technology). The printed physical form of the model can be referred to as a 3D model. Computer models can also be used in environments with models of other objects, people, or systems. Computer models can also be used to generate simulations, display in virtual environment systems, display in augmented reality systems, or the like. Computer models can be used in Computer Aided Design (CAD) and/or Computer Aided Manufacturing (CAM) systems. Certain models may be identified with an adjective that identifies the object, person, or system the model represents. For example, a “bone” model is a model of a bone, and a “heart” model is a model of a heart. (Search “model” on Wikipedia.com Jun. 13, 2021. CC-BY-SA 3.0 Modified. Accessed Jun. 23, 2021.) As used herein, “additive manufacturing” refers to a manufacturing process in which materials are joined together in a process that repeatedly builds one layer on top of another to generate a three-dimensional structure or object. Additive manufacturing may also be referred to using different terms including: additive processes, additive fabrication, additive techniques, additive layer manufacturing, layer manufacturing, freeform fabrication, ASTM F2792 (American Society for Testing and Materials), and 3D printing. Additive manufacturing can build the three-dimensional structure or object using computer-controlled equipment that applies successive layers of the material(s) based on a three-dimensional model that may be defined using Computer Aided Design (CAD) software. Additive manufacturing can use a variety of materials including polymers, thermoplastics, metals, ceramics, biochemicals, and the like. Additive manufacturing may provide unique benefits, as an implant together with the pores and/or lattices can be directly manufactured (without the need to generate molds, tool paths, perform any milling, and/or other manufacturing steps).

As used herein, “template instrument” refers to an instrument configured, designed, and/or engineered to serve as a template for creating, generating, or fabricating a patient-specific instrument. In one aspect, the template instrument may be used, as-is, without any further changes, modifications, or adjustments and thus become a patient-specific instrument. In another aspect, the template instrument may be modified, adjusted, or configured to more specifically address the goals, objectives, or needs of a patient or a surgeon and by way of the modifications become a patient-specific instrument. The patient-specific instrument can be used by a user, such as a surgeon, to guide making one or more resections of a structure, such as a bone for a procedure. Accordingly, a template instrument model can be used to generate a patient-specific instrument model. The patient-specific instrument model may be used in a surgical procedure to address, correct, or mitigate effects of the identified deformity and may be used to generate a patient-specific instrument that can be used in a surgical procedure for the patient.

Next, the method 300 may register 308 the preliminary instrument model with one or more bones of the bone model. This step 308 facilitates customization and modification of the preliminary instrument model to generate a patient-specific instrument model from which a patient-specific instrument can be generated. The registration step 308 may combine two models and/or patient imaging data and position both models for use in one system and/or in one model.

Next, the method 300 may design 310 a patient-specific instrument and/or procedure model based on the preliminary instrument model. The design step 310 may be completely automated or may optionally permit a user to make changes to a preliminary instrument model or partially completed patient-specific instrument model before the patient-specific instrument model is complete. A preliminary instrument model and patient-specific instrument model are two examples of an instrument model. As used herein, “instrument model” refers to a model, either physical or digital, that represents an instrument, tool, apparatus, or device. Examples, of an instrument model can include a cutting instrument model, a resection instrument model, an alignment instrument model, a reduction instrument model, a patient-specific tendon trajectory instrument model, graft harvesting instrument model, minimally invasive surgical (MIS) positioner model, or the like. In one embodiment, a patient-specific instrument and a patient-specific instrument model may be unique to a particular patient and that patient's anatomy and/or condition.

The method 300 may conclude by a step 312 in which a patient-specific instrument may be manufactured based on the patient-specific instrument model. Various manufacturing tools, devices, systems, and/or techniques can be used to manufacture the patient-specific instrument.

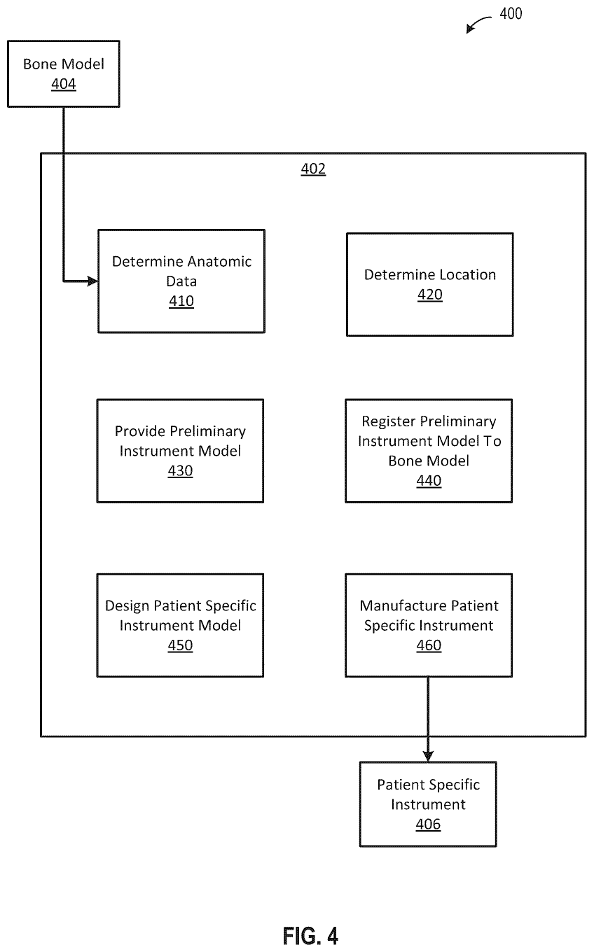

FIG. 4 illustrates an exemplary system 400 configured to generate one or more patient-specific instruments configured to facilitate surgical procedures, according to one embodiment. The system 400 may include an apparatus 402 configured to accept, review, receive or reference a bone model 404 and provide a patient-specific instrument 406. In one embodiment, the apparatus 402 is a computing device. In another embodiment, the apparatus 402 may be a combination of computing devices and/or software components or a single software component such as a software application.

The apparatus 402 may include a determination module 410, a location module 420, a provision module 430, a registration module 440, a design module 450, and a manufacturing module 460. Each of which may be implemented in one or more of software, hardware, or a combination of hardware and software.

The determination module 410 determines anatomic data 412 from a bone model 404. In certain embodiments, the system 400 may not include a determination module 410 if the anatomic data is available directly from the bone model 404. In certain embodiments, the anatomic data for a bone model 404 may include data that identifies each anatomic structure within the bone model 404 and attributes about the anatomic structure. For example, the anatomic data may include measurements of the length, width, height, and density of each bone in the bone model. Furthermore, the anatomic data may include position information that identifies where each structure, such as a bone is in the bone model 404 relative to other structures, including bones. The anatomic data may be in any suitable format and may be stored separately or together with data that defines the bone model 404.

In one embodiment, the determination module 410 may use advanced computer analysis system such as image segmentation to determine the anatomic data. The determination module 410 may determine anatomic data from one or more sources of medical imaging data, images, files, or the like. Alternatively, or in addition the determination module 410 may use software and/or systems that implement one or more artificial intelligence methods (e.g., machine learning and/or neural networks) for deriving, determining, or extrapolating, anatomic data from medical imaging or the bone model. In one embodiment, the determination module 410 may perform an anatomic mapping of the bone model 404 to determine each unique aspect of the intended osteotomy procedure and/or bone resection and/or bone translation. The anatomic mapping may be used to determine coordinates to be used for an osteotomy procedure, position and manner of resections to be performed either manually or automatically or using robotic surgical assistance, a width for bone cuts, an angle for bone cuts, a predetermined depth for bone cuts, dimensions and configurations for resection instruments such as saw blades, milling bit size and/or speed, saw blade depth markers, and/or instructions for automatic or robotic resection operations.

In one embodiment, the determination module 410 may use advanced computer analysis system such as image segmentation to determine the anatomic data. The determination module 410 may determine anatomic data from one or more sources of medical imaging data, images, files, or the like. The determination module 410 may perform the image segmentation using 3D modeling systems and/or artificial intelligence (AI) segmentation tools. In certain embodiments, the determination module 410 is configured to identify and classify portions of bone based on a condition of the bone, based on the bone condition. Such classifications may include identifying bone stability, bone density, bone structure, bone deformity, bone structure, bone structure integrity, and the like. Accordingly, the determination module 410 may identify portions or sections or one or more bones based on a quality metric for the bone. Advantageously, that determination module 410 can identify high quality bone having a viable structure, integrity, and/or density versus lower quality bone having a nonviable structure, integrity, and/or density and a plurality of bone quality levels in between.

Accordingly, the determination module 410 can guide a surgeon to determine which areas of one or more bones of a patient are within a “soft tissue envelope” (bone of undesirable quality) as that bone relates to a particular deformity or pathology. Identifying the quality of one or more bones of the patient can aid a surgeon in determining what type of correction or adjustment is needed. For example, an ulceration that occurs due to a boney deformity can be mapped using the determination module 410 in a way that a correction can be performed to correct the deformity and reduce pressure to an area and address the structures that were causing the pressure ulceration/skin breakdown.

In addition, the determination module 410 and/or another component of the apparatus 402 can be used to perform anatomic mapping which may include advanced medical imaging, such as the use of CT scan, ultrasound, MRI, X-ray, and bone density scans can be combined to effectively create an anatomic map that determines the structural integrity of the underlying bone.

Identifying the structural integrity of the underlying bone can help in determining where bone resections (e.g., osteotomies) can be performed to preserve the densest bone in relation to conditions such as Charcot neuropathic, arthropathy where lesser dense bone can fail and collapse. It is well documented in the literature that failure to address and remove such lesser dense bone can ultimately lead to failure of a reconstruction and associated hardware.

The present disclosure provides, by way of at least the exemplary system 400, an anatomic map that can be part of anatomic data. The anatomic map can combine structural, deformity, and bone density information and can be utilized to determine the effective density of bone and help to determine where bone should be resected in order to remove the lesser dense bone while maintaining more viable bone to aid in the planning of the osteotomy/bone resection placement.

The location module 420 determines or identifies one or more recommended locations and/or trajectory angles for deployment of an instrument and/or soft tissue based on the anatomic data 412 and/or the bone model 404. In one embodiment, the location module 420 may compare the anatomic data 412 to a general model that is representative of most patient's anatomies and may be free from deformities or anomalies. The location module 420 can operate autonomously and/or may facilitate input and/or revisions from a user. The location module 420 may be completely automated, partially automated, or completely manual. A user may control how automated or manual the determining of the location and/or trajectory angles is.

The provision module 430 is configured to provide a preliminary instrument model 438. The provision module 430 may use a variety of methods to provide the preliminary instrument model. In one embodiment, the provision module 430 may generate a preliminary instrument model. In the same, or an alternative embodiment, the provision module 430 may select a template instrument model for a tendon (or tendon substitute) deployment procedure configured to enable locating the position and/or providing the trajectory provided by the location module 420. In one embodiment, the provision module 430 may select a template instrument model for a minimally invasive surgical (MIS) bunion correction procedure configured to enable locating the position and/or providing the trajectory for the fixation deployment. In one embodiment, the provision module 430 may select a template instrument model from a set of template instrument models (e.g., a library, set, or repository of template instrument models).

The registration module 440 registers the preliminary instrument model with one or more bones or other anatomical structures of the bone model 404. As explained above, registration is a process of combining medical imaging data, patient imaging data, and/or one or more models such that the preliminary instrument model can be used with the bone model 404.

The design module 450 designs a patient-specific instrument (or patient-specific instrument model) based on the preliminary instrument model. The design operation of the design module 450 may be completely automated, partially automated, or completely manual. A user may control how automated or manual the designing of the patient-specific instrument (or patient-specific instrument model) is.

The manufacturing module 460 may manufacture a patient-specific instrument 406 using the preliminary instrument model. The manufacturing module 460 may use a patient-specific instrument model generated from the preliminary instrument model. The manufacturing module 460 may provide the patient-specific instrument model to one or more manufacturing tools and/or fabrication tool (e.g., additive and/or subtractive). The patient-specific instrument model may be sent to the tools in any format such as an STL file or any other CAD modeling or CAM file or method for data exchange. In one embodiment, a user can adjust default parameters for the patient-specific instrument such as types and/or thicknesses of materials, dimensions, and the like before the manufacturing module 460 provides the patient-specific instrument model to a manufacturing tool.

Effective connection of the guide to one or more bones can ensure that surgical steps are performed in desired locations and/or with desired orientations and mitigate undesired surgical outcomes.

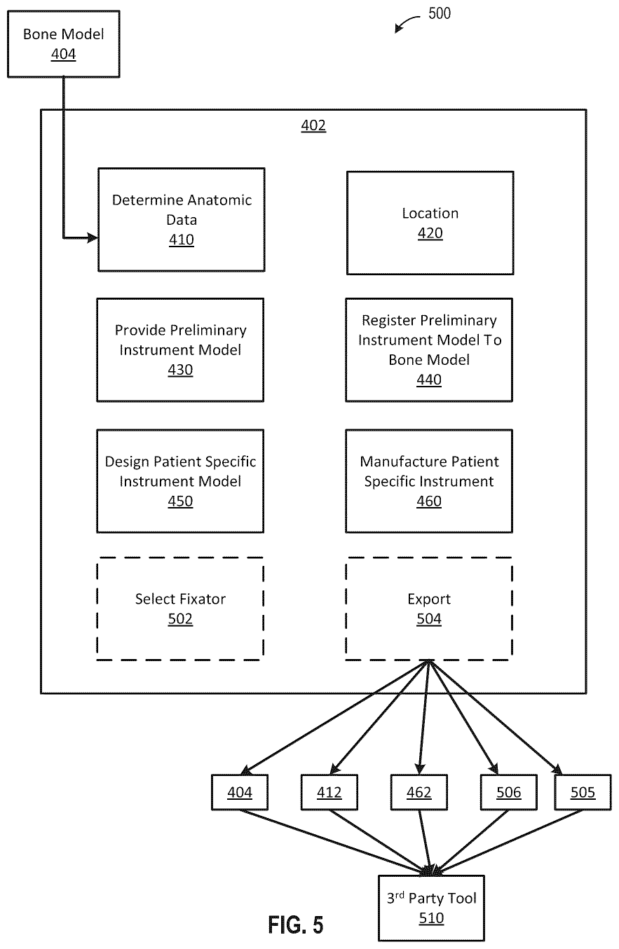

FIG. 5 illustrates an exemplary system 500 configured to generate one or more patient-specific instruments configured to correct a bone condition, according to one embodiment. The system 500 may include similar components or modules to those described in relation to FIG. 4. In addition, the system 500 may include a fixator selector 502 and/or an export module 504.

The fixator selector 502 enables a user to determine which fixator(s) to use for a MIS bunion correction procedure planned for a patient. In one embodiment, the fixator selector 502 may recommend one or more fixators based on the bone model 404, the location, the trajectory, or input from a user or a history of prior MIS bunion correction procedures performed. The fixator selector 502 may select a fixator model from a set of predefined fixator models or select a physical fixator from a set of fixators. The fixators may include a plate and associated accessories such as screws, anchors, and the like.

In one embodiment, the fixator selector 502 includes an artificial intelligence or machine learning module. The artificial intelligence or machine learning module is configured to implement one or more of a variety of artificial intelligence modules that may be trained for selecting fixator(s) based on anatomic data 412 and/or other input parameters. In one embodiment, the artificial intelligence or machine learning module may be trained using a large data set of anatomic data 412 for suitable fixator(s) identified and labeled in the dataset by professionals for use to treat a particular condition. The artificial intelligence or machine learning module may implement, or use, a neural network configured according to the training such that the artificial intelligence or machine learning module is able to select or recommend suitable fixator(s).

The export module 504 is configured to enable exporting of a patient-specific instrument model 462 for a variety of purposes including, but not limited to, fabrication/manufacture of a patient-specific instrument 406 and/or fixator(s), generation of a preoperative plan, generation of a physical bone model matching the bone model 404, and the like. In one embodiment, the export module 504 is configured to export the bone model 404, anatomic data 412, a patient-specific instrument model 462, a preoperative plan 506, a fixator model 508, or the like. In this manner the custom instrumentation and/or procedural steps for a procedure (e.g., a graft harvesting procedure, minimally invasive surgical (MIS) procedure, or the like) can be used in other tools. The preoperative plan 506 may include a set of step by step instructions or recommendations for a surgeon or other staff in performing a procedure (e.g., a graft harvesting procedure, minimally invasive surgical (MIS) procedure, or the like). The preoperative plan 506 may include images and text instructions and may include identification of instrumentation to be used for different steps of the procedure (e.g., a graft harvesting procedure, minimally invasive surgical (MIS) procedure, or the like). The instrumentation may include the patient-specific instrument 406 and/or one or more fixators/fasteners. In one embodiment, the export module 504 may provide a fixator model which can be used to fabricate a fixator for the procedure.

The exports (404, 412, 462, 506, and 508) may be inputs for a variety of 3rd party tools 510 including a manufacturing tool, a simulation tool, a virtual reality tool, an augmented reality tool, an operative procedure simulation tool, a robotic assistance tool, and the like. A surgeon can then use these tools when performing a procedure or for rehearsals and preparation for the procedure. For example, a physical model of the bones, patient-specific instrument 406, and/or fixators can be fabricated, and these can be used for a rehearsal operative procedure. Alternatively, a surgeon can use the bone model 404, preliminary instrument model 438, and/or a fixator model to perform a simulated procedure using an operative procedure simulation tool.

Referring now to FIGS. 3-5, certain methods, systems, and/or apparatuses are disclosed herein for preparing for, planning, outlining, and/or instrumenting, one or more surgical procedures. Alternatively, or in addition, the methods, systems, and/or apparatuses a disclosed herein can be used for preoperative development and design of systems, instrumentation, and/or implants and/or for preoperative rehearsal and/or instruction of a surgeon before the surgical procedure. For example, a surgeon can use the method 300, bone model(s) 404, patient instrument(s) 406, system 400, and/or apparatus 402 to perform a mock surgical procedure virtually before an actual surgical procedure.

These techniques and/or technologies can greatly advance the medical field and provide valuable instruction and experience to a surgeon prior to an actual surgical procedure. Furthermore, these techniques and/or technologies are made effective owing to the accuracy and precision of the models because of the fidelity of the medical imaging of the patient anatomy. This virtual modeling of patient anatomy has become very accurate and helpful, particularly for hard tissue such as bones and the surfaces of these bones.

Unfortunately, the fidelity and accuracy of these models is not as advanced with respect to the modeling of soft tissue of a patient such as sinews, skin, tendons, ligaments, muscles, fat, and the like. Thus, rehearsal of a surgical procedure, particularly one that includes translating and/or reorienting one or more bone fragments may have limited benefits. In such cases, because the surgeon cannot predict or know beforehand how much movement and reorientation the soft tissue of a patient will permit, the surgeon may need to revise or adapt a surgical procedure intraoperatively to achieve optimal outcomes. The system, apparatus, and methods of the present disclosure enable a surgeon to make intraoperative adjustments to surgical plan based on what the surgeon learns during the surgery.

The present disclosure leverages the use of models, such as computer models, and particularly models of a specific patient to provide and/or generate instrumentation, implants, and/or surgical plans that advanced patient care. Advantageously, these models are unique and customized for a particular patient. Thus, the models reflect the actual anatomical features and aspects of the patient.

However, the utility and helpfulness of the models, methods, systems, and/or apparatuses of FIGS. 3-5, may be dependent on how effectively a surgeon can navigate within, on, or in relation to one or more anatomical references or anatomical features of a patient such that the steps of the surgical procedure can be performed on a patient in the same manner as those modeled using models of the anatomy of the patient. This process of navigation is referred to as a mapping or translation between the virtual or model environment to a physical or real world environment that includes the patient anatomy and the operating field.

Advantageously, the models, methods, systems, and/or apparatuses of the present disclosure facilitate mapping or translating between a virtual or model environment and/or instrumentation to a physical or real world environment for a surgical procedure. The present disclosure provides this feature or benefit by providing an apparatus, system, and method, that enables a surgeon to identify, create, form, and/or use reference features for a surgical procedure. The reference feature provides a reference and/or starting point on, in, or associated with anatomy of a patient such that steps, stages, features, or aspects planned and configured within the model can be accurately performed on, with, or to the anatomy of the patient. In certain embodiments, one or more steps of a surgical procedure can be done in connection with or in relation to the reference feature.

The reference feature facilitates moving from one coordinate system or frame of reference in a virtual environment to a position, location, frame of reference, environment, or orientation on, or in, an actual object, structure, device, apparatus, anatomical structure, or the like. Advantageously, the reference feature can coordinate objects, models, or structures in a digital or virtual model or representation with corresponding objects or structures (e.g., anatomical structures) of actual physical objects or structures. Said another way, the reference feature can serve to map from a virtual or modeled object to an actual or physical object.

Advantageously, the embodiment of the present disclosure include features and aspects that assist a surgeon in locating at least one reference feature, which can then be used in one or more stages of a surgical procedure. In certain embodiments, the actual instruments fabricated using the present disclosure may include one or more references (e.g., a model references). The one or more model instruments may use the one or more references to position and/or orient the one or more model instruments such that other steps of a surgical procedure can be performed in relation to those one or more model instruments and/or model references. Certain model references may key off or related to anatomical references of modeled anatomical body parts. The reference feature(s) correspond to the model references and together enable a surgeon to identify reference features on actual anatomy of a patient for a surgical procedure.

In certain embodiments, one or more fasteners deployed in an instrument such as a resection guide can serve as reference features, for an initial stage of the surgical procedure and/or for subsequent stages of the surgical procedure. In certain embodiments, a bone engagement feature can serve as a reference feature for an osteotomy system and/or surgical procedure.

Advantageously, the embodiments of the present disclosure leverage patient-specific models of patient anatomy and the use of these models to generate patient-specific instruments as well as input from users of the osteotomy (e.g., surgeons). In one embodiment, this input is provided in the form of user directions. Combining patient-specific medical imaging, patient-specific anatomical models, and user directions enable the present disclosure to provide a customized or patient-specific osteotomy that serves the patient's needs as well as aides the surgeon in performing the surgical procedure. In this manner, a surgeon can perform the surgical procedure with higher confidence and assurance that the procedure performed on the patient will coincide with the plan set forth using models in a virtual environment. Consequently, the present disclosure improves the level of patient care and positive outcomes.

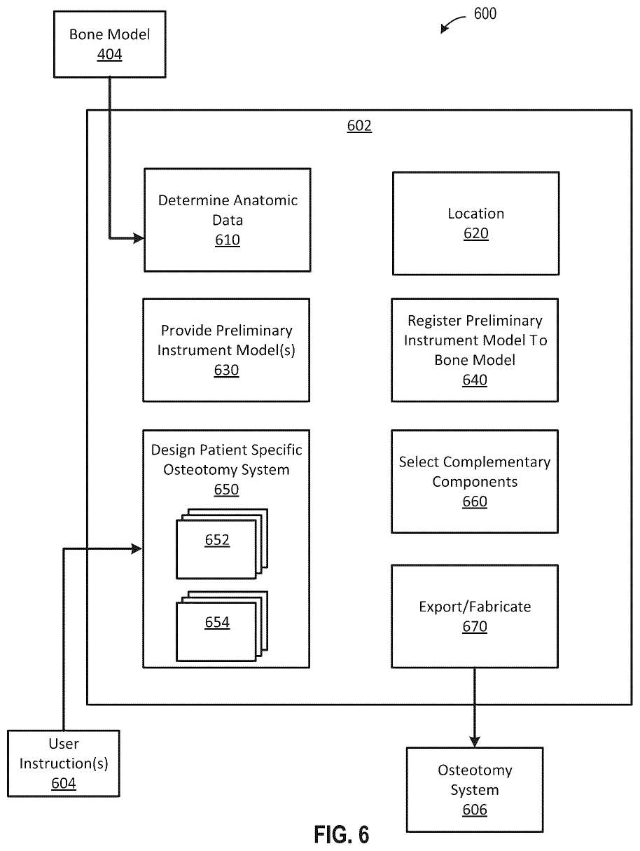

FIG. 6 illustrates an exemplary system 600 configured to design, generate, develop, and/or produce an osteotomy system, according to one embodiment. In certain embodiments, the osteotomy system can be patient-specific. One advantage of the present disclosed embodiments is that an end user of an osteotomy system (e.g., instruments, preoperative plan, implants, etc.) can have as much, or as little control or input, over one or more or all of the aspects of the osteotomy system. Furthermore, this osteotomy system can be customized both to the needs and specific aspects of the patient as well as to the needs and/or preferences and/or desires of the user (e.g., surgeon).

The system 600 may include similar components or modules to those described in relation to FIG. 4. The structures, features, and functions, operations, and configurations of the system 600 may be similar or identical to components or modules of system 400, like parts identified with similar reference numerals. Accordingly, the system 600 may include an apparatus 602 configured to accept, review, receive or reference a bone model 404 and user instructions 604 and provide a patient-specific osteotomy system 606. In one embodiment, the apparatus 602 is a computing device. In another embodiment, the apparatus 602 may be a combination of computing devices, systems, apparatuses, software components, single software component such as a software application, one or more third party manufacturers, or the like.

The apparatus 602 may include a determination module 610, a location module 620, a provision module 630, an optional registration module 640, a design module 650, a selection module 660, and an export/fabrication module 670. Each of which may be implemented in one or more of, software, hardware, or a combination of hardware and software. In certain embodiments, one or more parts of the system 600 may be operated by a user (e.g., a technician), a plurality of users, and may include input, involvement, and/or feedback from an end user of the osteotomy system developed. Generally, the end user of the osteotomy system will be a surgeon. Those of skill in the art will appreciate that depending on the surgical procedure being performed, one or more of the modules of the apparatus 602 may or may not be used.

The determination module 610 may operate in a similar manner to the determination module 410. The location module 620 may operate in a similar manner to the location module 420. The provision module 630 may operate in a similar manner to the provision module 430. The registration module 640 may operate in a similar manner to the registration module 440.

The design module 650 enables one or more users to design an osteotomy system 606 and in particular a patient-specific osteotomy system 606. A patient-specific osteotomy system 606 can include a number of different instruments, components, and/or systems, including but not limited to one or more cutting tools, one or more resection guides, one or more provisional fasteners, one or more fixation systems and/or instruments, a preoperative plan, one or more kits of implants and/or trial components, one or more alignment guides, one or more positioning guides, one or more reduction guides, one or more, one or more navigation guides, one or more fixation guides, one or more, one or more compression guides, one or more rotation guides, and the like. In addition, one or more of these components can be patient-specific. For example, the patient-specific osteotomy system 606 can include a patient-specific instrument, patient-specific trajectory guide, a patient-specific resection guide, a patient-specific cutting guide, a patient-specific positioning guide or positioner, another patient-specific instrument, or the like.

Alternatively, or in addition, the patient-specific osteotomy system 606 can include one or more subparts or components of each of the instruments, components and/or systems of the patient-specific osteotomy system 606. For example, in one embodiment, the design module 650 may enable a user and/or end user to determine and/or define a number, size, shape, position, orientation, trajectory and/or configuration for one or more bone attachment features, a number, size, shape, position, orientation, trajectory and/or configuration for one or more resection features, a number, size, shape, position, orientation, trajectory and/or configuration for one or more bone engagement features, a number, size, shape, position, orientation, trajectory and/or configuration for one or more bone engagement surfaces, a number, size, shape, position, orientation, trajectory and/or configuration for one or more fixators (either or both provisional or permanent), and the like.

Those of skill in the art will appreciate that the design module 650 offers a large variety of different options and combinations for the constituents of the patient-specific osteotomy system 606 as well as a plurality of options for the components of the patient-specific osteotomy system 606 and that such options may be overwhelming. Advantageously, the surgical procedure to be performed, the bone model 404, and user instructions 604 each alone and/or in combination define an initial set of members for the patient-specific osteotomy system 606. For example, certain well known surgical techniques have specific names and surgeons understand and/or have experience doing these procedures and therefore know what instruments will be needed for the surgical procedure.

In addition, each surgeon is different just as each patient is different. Therefore, surgeon experience and/or preferences may factor into the members of the patient-specific osteotomy system 606 a particular surgeon wants and/or the configuration of the members of the patient-specific osteotomy system 606. For example, where one surgeon may prefer to use two resection guides another surgeon may want to use one resection guide and perform other osteotomies for the surgical procedure manually or free-hand.

Based on the surgical procedure to be performed, many decisions about the design and/or make up of the patient-specific osteotomy system 606 can be made as recommendations and/or proposals by a technician to a surgeon. These decisions can be based in whole or in part on the surgical procedure to be performed, the bone model 404 and/or the user instructions 604.

For example, suppose a surgeon would like a patient-specific osteotomy system 606 for an osteotomy procedure. One goal of the procedure may be to relieve pain of the patient and to remove a minimal amount of bone in the process of completing the procedure. In such an example, the bone model 404 may be of one or more bones of a foot and/or ankle of the patient. The surgeon may provide a request and/or a set of user instructions 604 for a patient-specific osteotomy system 606 for this osteotomy procedure.

Those of skill in the art will appreciate that the user instructions 604 may be of a variety of different types, lengths, number of details and may be provided in a variety of different formats including oral, written, or the like. In one embodiment, the user instructions 604 may be a request for a patient-specific osteotomy system 606 that includes a set of default instruments, preoperative plan, implants, or the like. For example, the user instructions 604 may as short and simple as “Please provide an osteotomy system for a bone of the left foot for patient <<identifying information (e.g., name, dob, etc.)>> with medial approach.” The user instructions 604 may be provided in the form of a product order, a purchase order, a prescription, or the like. The user instructions 604 may be provided in written manual/analog form, include a manual signature, digital form, include an e-signature, or the like. In addition, the user instructions 604 may include security and/or authorization features that enable the receiver to confirm that the user instructions 604 are valid and are authorized by a particular surgeon or doctor. The user instructions 604 may indicate the approach to the surgical site (e.g., an ankle or foot joint or bone) the surgeon wants to take, anterior, posterior, medial, lateral, or the like.

In another embodiment, the user instructions 604 may include specific instructions for the number and/or kind or type of components in the patient-specific osteotomy system 606 and/or the configuration of one or more of these components. For example, the user instructions 604 may identify a specific fixation product or fixation system the surgeon will be using for permanent fixation of the osteotom(ies). Alternatively, or in addition, the user instructions 604 may include designation of one or more complementary components and/or configurations for these components to be included in the patient-specific osteotomy system 606.

In one embodiment, the user instructions 604 may designate a particular material and/or mass for fabricating one or more guides to be included in the patient-specific osteotomy system 606. Some surgeons may find that patient-specific instruments, such as a patient-specific resection guide may more readily register to one or more bone surfaces if the instrument has a greater mass and/or weight. With the greater mass and a sufficient fidelity bone engagement surface, a patient-specific instrument may seem to find its own way or seek out a desired position on a bone that matches or substantially matches a position planned when the patient-specific osteotomy system 606 was developed. Consequently, a surgeon may request in the user instructions 604 that the instrument be made from a metal such as titanium.

With the bone model 404 and user instructions 604 a user such as a technician may operate the design module 650 alone or together with other modules of the apparatus 602 to develop a patient-specific osteotomy system 606. In certain embodiments, a single user operates the apparatus 602. Alternatively, or in addition, a plurality of users, which may include an end user, such as surgeon can operate or interact with one or more modules of the apparatus 602 as the patient-specific osteotomy system 606 is designed or developed.

In one embodiment, a technician may provide a patient-specific osteotomy system 606 that includes one or more complementary components and one or more resection guides, which may be patient-specific. The technician may also provide a preoperative plan. These may be provided to an end user (e.g., surgeon) either directly or by accessing the apparatus 602 remotely. The end user may review the preoperative plan and/or the components of the patient-specific osteotomy system 606 (e.g., resection guides) and may approve of the patient-specific osteotomy system 606 or may request changes. In certain embodiments, these changes may include the addition of one or more added bone engagement features, one or more resection guides, a change in a trajectory for a bone attachment feature, a change in trajectory for an osteotomy, an addition of openings in a guide to coincide with openings needed for a fixation system, as well as a plurality of other possible changes to the patient-specific osteotomy system 606. The technician may then make the requested changes and present a revised patient-specific osteotomy system 606 for the surgeon to review again. Next, the surgeon may approve of the revised patient-specific osteotomy system 606 and/or request additional changes.

In the illustrated embodiment, the design module 650 may include a plurality of resection features 652 and/or a plurality of bone engagement features 654. A technician may select one or more resection features 652 and/or one or more bone engagement features 654 and include them in the patient-specific osteotomy system 606. Alternatively, or in addition, a surgeon may designate which resection features 652 and/or bone engagement features 654 to include in the patient-specific osteotomy system 606.

In certain embodiments, the surgeon and technician may collaborate and/or consult with each other regarding the design and/or configuration of the patient-specific osteotomy system 606 and its components. The technician may share with the surgeon information about the technological features and/or limitations of the components of the patient-specific osteotomy system 606 and use the technician's experience and know-how to make recommendations to the surgeon. The surgeon can present ideas and/or requests regarding what the surgeon would like for components of the patient-specific osteotomy system 606 and the technician can determine whether those ideas/requests can be satisfied using a patient-specific osteotomy system 606.

The apparatus 602 uses both the bone model 404 and user instructions 604 to provide a patient-specific osteotomy system 606. Advantageously, the apparatus 602 enables a surgeon to be involved in the design and development of a patient-specific osteotomy system 606 that is suited not just for the patient, but also for the needs, skills and/or preferences of the surgeon. In this manner, a patient-specific osteotomy system 606 can be provided that improves patient care and accomplishing of desired outcomes.

In one embodiment, the operation of the design module 650 may be completely automated, partially automated, or completely manual. A user may control how automated or manual the designing of the patient-specific osteotomy system 606 is, including patient-specific instrument models, patient-specific instruments, and/or other components of the patient-specific osteotomy system 606.

The apparatus 602 may include a selection module 660 and an export/fabrication module 670. The selection module 660 facilitates the selection and/or customization of one or more complementary components for a patient-specific osteotomy system 606. Complementary components are described herein, but can include certain guides or other aids to facilitate completing a surgical procedure as planned. In one embodiment, the operation of the selection module 660 may be completely automated, partially automated, or completely manual. A user may control how automated or manual the selection module 660 is.

The export/fabrication module 670 is configured to enable exporting of a patient-specific osteotomy system 606 for a variety of purposes including, but not limited to, fabrication/manufacture of one or more patient-specific instruments and/or fixator(s), ordering or fabricating one or more members of the patient-specific osteotomy system 606, generation of a preoperative plan, generation of a physical bone model matching the bone model 404, and the like.

In one embodiment, the export/fabrication module 670 is configured to export the bone model 404, anatomic data 412, one or more patient-specific instrument models 462, a preoperative plan 506, a fixator model 508, or the like. In this manner the custom instrumentation and/or procedural steps for a procedure can be used in other tools. The preoperative plan 506 may include a set of step by step instructions or recommendations for a surgeon or other staff in performing a procedure (e.g., a graft harvesting procedure, minimally invasive surgical (MIS) procedure, or the like). The preoperative plan 506 may include images and text instructions and may include identification of instrumentation to be used for different steps of the procedure (e.g., a graft harvesting procedure, minimally invasive surgical (MIS) procedure, or the like). The instrumentation may include a patient-specific instrument, bone engagement features, and/or one or more fixators/fasteners.

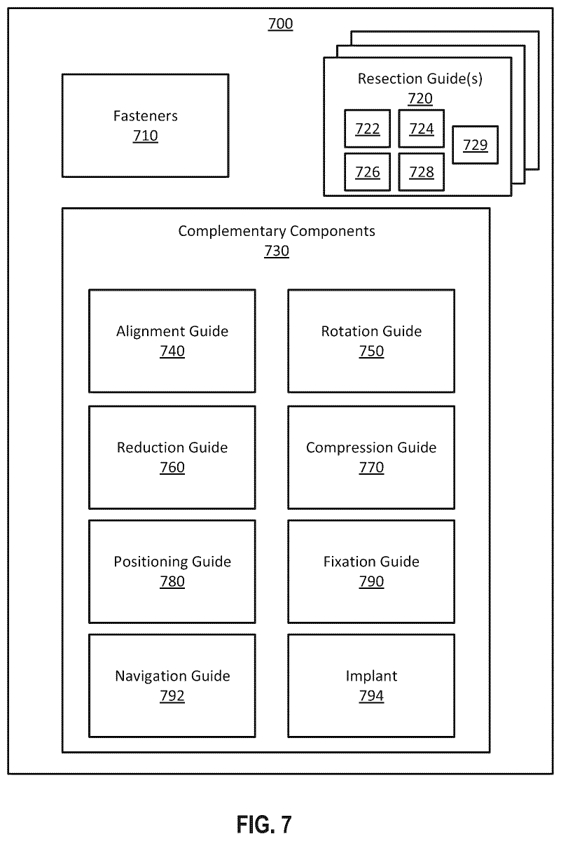

FIG. 7 illustrates an exemplary system 700 for remediating a condition present in a patient's foot, according to one embodiment. The system 700 can include one or more fasteners 710, one or more resection guides 720, and one or more complementary components 730. While a system 700 can be used for a variety of procedures, one or more features, components, and/or aspects of the system 700 may be particularly suited for one or more osteotomies on one or more bones of a structure such as a patient's foot, ankle, wrist, hand, shoulder, or the like.

In certain embodiments, the one or more fasteners 710 can include one or more permanent fasteners and/or one or more temporary fasteners. Typically, the fasteners 710 may be used during a variety of different steps of a procedure. Temporary fasteners are often used because they can securely hold bone or parts/fragments of bones while steps of the procedure are conducted. A common temporary fastener that can be used with system 700 is a K-wire, also referred to as a pin, guide pin, and/or anchor pin. Permanent fasteners 710 such as bone screws, bone staples, sutures, tethers or the like may also be used in a surgical procedure.

The one or more resection guides 720 assist a surgeon in performing different resection or dissection steps for an osteotomy or other procedure. In certain embodiments, a resection guide 720 includes one or more resection features 722 and one or more bone attachment features 724. The resection features 722 can take a variety of forms and/or embodiments. In one embodiment, the resection features 722 take the form of a cut channel or slot or other opening.

The resection features 722 provide a guide for a surgeon using a cutting tool to resect a bone, one or more bones, or other tissues of a patient. In certain embodiments, the resection features 722 may guide a surgeon in performing a resection, and osteotomy, and/or a dissection.

Similarly, the bone attachment features 724 can take a variety of forms and/or embodiments. The bone attachment features 724 may serve to secure the resection guide 720 and/or other instrumentation to one or more bones and/or one or more other structures. Often, a bone attachment feature 724 can take the form of a hole in and/or through the resection guide 720 together with a temporary fastener such as a K-wire, pin, or guide pin.

The bone attachment features 724 facilitate attachment (at least temporarily) of a resection guide 720 to one or more bones, or bone fragments, of a patient. The bone attachment features 724 may include any of a wide variety of fasteners or structures including, but not limited to, holes, spikes, prongs, screws, fastening devices, and/or the like. Effective connection of the resection guide 720 to one or more bones across a joint and/or to one or more bones can ensure that cut surfaces are formed in desired locations and orientations and mitigate removal of hard tissue and/or soft tissue in undesired locations and/or orientations.

In certain embodiments, a resection guide 720 may include one or more bone engagement surfaces 726 and/or one or more landmark registration features 728. In certain embodiments, a landmark registration feature 728 may extend from one or more sides or ends of a resection guide 720 and engage with one or more landmarks of a bone or joint or anatomical structure of a patient. Registration of the landmark registration feature 728 to a landmark of a bone or joint can serve to confirm and/or ensure that a surgeon has located a desired placement and/or orientation for a resection guide 720.

In certain embodiments, the bone engagement surfaces 726 are patient-specific: contoured to match a surface of: one or more bones and/or bone surfaces the resection guide 720 contacts during the procedure or one or more joints proximal to the resection guide 720 during the procedure. Alternatively, or in addition, the bone engagement surface 726 may not be patient-specific, and may, or may not, contact a bone surface during use of the resection guide 720. In one embodiment, a skin contact surface may be used in addition to, or in place of, a bone engagement surface. Those of skill in the art appreciate that one or more sides of any of the members of the system 700 may include one or more bone engagement surfaces 726. Consequently, one or more sides of the fasteners 710, the resection guide(s) 720, the complementary components 730, navigation guides 792, and/or the implants 794 may include one or more bone engagement surfaces 726.

In certain embodiments, the resection guides 720 and/or aspects of the resection guides 720 may be integrated into other components and/or instruments, such as a pin guide, a trajectory guide, an alignment guide, or the like.

The complementary components 730 serve to assist a surgeon during one or more steps of a procedure. Those of skill in the art appreciate that a number of components can serve as complementary components 730. One or more of the features, functions, or aspects of the complementary components 730 can include patient-specific features.

Examples of complementary components 730 include, but are not limited to, an alignment guide 740, a rotation guide 750, a reduction guide 760, a compression guide 770, a positioning guide 780, a fixation guide 790, navigation guides 792, and/or one or more implants 794. In general, the complementary components 730 serve to assist a surgeon in performing the function included in the name of the complementary component 730. Thus, an alignment guide 740 can help a surgeon align bones, parts of bones, or other parts of a patient as part of a procedure. A rotation guide 750 can help a surgeon rotate one or more bones, parts of bones, or other parts of a patient as part of a procedure. In one embodiment, a rotation guide 750 may hold one bone fragment stable while another bone fragment is rotated into a desired position.

A reduction guide 760 can help a surgeon position and/or orient one or more bones, parts of bones, or other parts of a patient as part of a procedure in order to reduce the bone, bones, bone parts, or other parts and/or in order to position and/or orient the bone, bones, bone parts, or other parts to a desired position and/or orientation. In certain embodiments, aspects and/or features of a reduction guide 760 can be integrated into one or more other components of an osteotomy system 700, such as components of the complementary components 730. A compression guide 770 can help a surgeon compress one or more bones, parts of bones, or other parts of a patient together or against an implant as part of a procedure. In certain embodiments, compression guide 770 can be a separate instrument such as a compressor and/or a combined compressor/distractor. The compressor/distractor can be used to compress two or more cut faces formed by an osteotomy until fixation is deployed or distract bones or parts of bones involved in a procedure. In certain embodiments, a compression guide 770 may serve a dual purpose as both a compression guide 770 and as a positioning guide 780. The same instrument may be used to both translate and/or rotate bones or bone fragments and compress two or more cut faces formed by an osteotomy until fixation can be deployed.

A positioning guide 780 (also referred to as a positioner) can help a surgeon position one or more bones, parts of bones, or other parts of a patient as part of a procedure. For example, a positioning guide 780 may hold one bone or bone fragment stable and hold one or more other bone fragments in a desired position while permanent or temporary fixation is deployed. In certain embodiments, the positioning guide 780 may hold bone fragments in a reduced position, and thus may function as both a positioning guide 780 and/or a reduction guide 760.

In certain embodiments, the positioning guide 780 may be designed and fabricated to be patient-specific. The patient-specific aspects can include a patient-specific bone engagement surface, a predefined angle for reorienting one or more bone or bone parts within one or more planes, a predefined position for bone attachment features 724 or fasteners 710, a predefined or patient-specific offset or amount of translation that is provided, or the like. Alternatively, or in addition, the positioning guide 780 may be selected from a kit, collection, or repository of a number of positioning guides 780: each having a different configuration for one or more aspects/attributes of the positioning guide 780. For example, each member of the repository/kit may include a different positioning angle (repositioning or correction angle), the angles may differ by 2 degrees for example. In such an embodiment, each positioning guide 780 may not be patient-specific to a particular patient but may provide the desired amount of positioning to meet the goals of the surgeon. In certain embodiments, a preoperative plan generated based on the present disclosure may include a recommendation for the positioning guide 780 to be used, even if the recommended positioning guide 780 is not patient-specific to the particular patient.

A fixation guide 790 can help a surgeon in completing one or more temporary or permanent fixation steps for one or more bones, parts of bones, or other parts of a patient as part of a procedure. The fixation guide 790 may include and/or may use one or more components of a fastener or fixation system including implant hardware of the fastener or fixation system.

Those of skill in the art will appreciate that the other complementary components 730 may each have functions, purposes, and/or advantages with respect to one or more anatomical parts of the patient. Alternatively, or in addition, the other complementary components 730 may each have functions, purposes, and/or advantages with respect to one or more instruments and/or one or more anatomical parts of the patient. For example, a trajectory guide may be a type of alignment guide 740 in that the trajectory guide facilitates alignment of fixation with the desired location and/or trajectory/orientation with respect to one or more anatomical parts of the patient. Alternatively, or in addition, a trajectory guide may also be considered a type of fixation guide 790 because the trajectory guide facilitates deployment of one or more fasteners 710.

Advantageously, the system 700 can help a surgeon overcome one or more of the challenges in performing an osteotomy procedure, particularly on bones of a hand or of a foot of a patient, such as on the forefoot, midfoot, or hindfoot. One challenge during an osteotomy procedure can be maintaining control of, and/or position, and/or orientation of a bone, one or more bones, and/or bone pieces/fragments, particularly once a resection or dissection is performed. Advantageously, the fasteners 710, resection guide(s) 720, and/or complementary components 730 can be configured to assist in overcoming this challenge.

Advantageously, system 700 can help a surgeon in positioning, placing, and/or orienting a resection guide accurately. Modern techniques may include preoperative planning, simulation, or even practice using computer models, 3D printed models, virtual reality systems, augmented reality systems or the like. However, simulations and models are still different from actually positioning a resection guide on a patient's bone, joint, or body part during the procedure. System 700 can include a number of features, including patient-specific features, to assist the surgeon with the positioning. In one embodiment, the resection guide 720 can include one or more landmark registration features 728.

Advantageously, the system 700 can help a surgeon in securing guides of the osteotomy system 700, such as a resection guide, as well as how to readily remove the guide (e.g., resection guide) without disturbing a reduction, shifting, reorienting, or repositioning one or more bones or parts of bones while removing the guide. In certain embodiments, the system 700 is configured to permit removal of a guide while keeping temporary fasteners in place for use in subsequent steps of an osteotomy procedure. Alternatively, or in addition, system 700 may facilitate positioning of temporary fasteners during one step of a wedge osteotomy procedure for use in a subsequent step of the wedge osteotomy procedure. Removal of a guide during an osteotomy procedure can be particularly challenging where translation and/or rotation of the bones involved in the osteotomy procedure is required for the success of the osteotomy procedure. Advantageously, system 700 accommodates translation and/or rotation of the bones during the osteotomy procedure while facilitating a successful outcome for the osteotomy procedure.

Advantageously, the components of the system 700 can be specifically designed for a particular patient. Alternatively, or in addition, the components of the system 700 can be specifically designed for a class of patients. Each of the components of system 700 can be designed, adapted, engineered and/or manufactured such that each feature, attribute, or aspect of the component is specifically designed to address one or more specific indications present in a patient. Advantageously, the cuts made for the osteotomy procedure can be of a size, position, orientation, and/or angle that provides for an optimal osteotomy with minimal risk of undesirable resection. In one embodiment, the components of system 700 can be configured such that an osteotomy is performed that enables a correction in more than one plane in relation to the parts of the body of the patient. For example, cut channels or resection features 722 in a resection guide 720 can be oriented and configured such that when the bones are fused/fixated the correction results from translation, rotation, and/or movement of bones or bone parts in two or more planes (e.g., sagittal and transverse) once the fragments or bones are reduced.

In certain embodiments, the exemplary system 700 may include a plurality of fasteners 710, resection guides 720, and/or complementary components 730. For example, a surgeon may plan to resect a plurality of osteotomies from the bone(s) in order to accomplish a desired correction. In one example, one or more wedge segments may be resected from a medial side of a patient's foot and another one or more wedge segments may be resected from a lateral side of the patient's foot. These wedge segments may extend part way into the foot, or through from one side of the foot to the other. Of course, multiple wedge segments may be formed on one side of the foot as well.

Additionally, a surgeon may use one or more components in an exemplary system 700 to make multiple cuts in the bone(s). The multiple cuts may be centered over or around an apex of a deformity or positioned at other locations within the foot such that when the multiple cuts are made, any resected segments removed, or added bone void fillers introduced, and/or bones and/or bone fragments translated and/or rotated the combined angles, surfaces, removed segments, and/or added portions cooperate to provide a desired correction. Each of the components of the exemplary system 700 can be identified, defined, and reviewed using the apparatuses, systems, and/or methods of the present disclosure.

In certain embodiments, the components of system 700 may be made as small as possible to minimize the amount of soft tissue that is opened in the patient for the osteotomy procedure. Alternatively, or in addition, walls and/or sides of the components may be beveled and/or angled to avoid contact with other hard tissue or soft tissues in the operating field for the osteotomy procedure.

Those of skill in the art will appreciate that for certain osteotomy procedures a complementary component 730 may not be needed or a given complementary component 730 may be optional for use in the osteotomy procedure. Similarly, those of skill in the art will appreciate that certain features of the fasteners 710, resection guides 720, and/or complementary components 730 can be combined into one or more of apparatus or devices or may be provided using a plurality of separate devices.

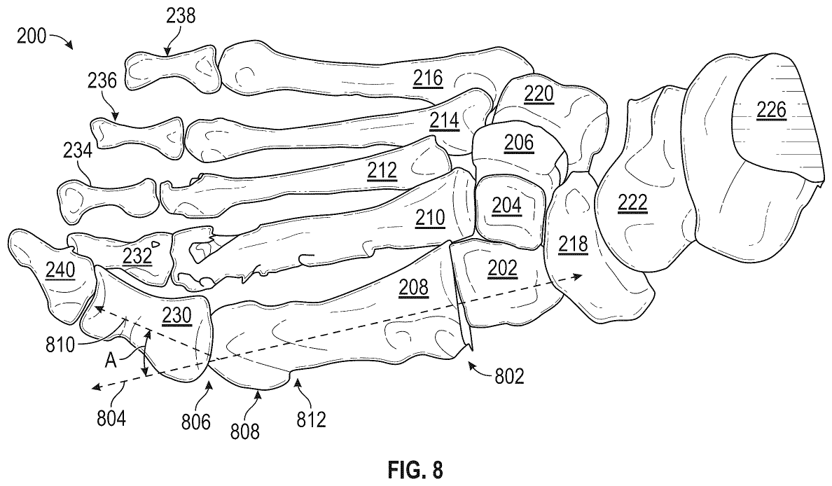

FIG. 8 illustrates exemplary bones of a patient with a bone condition suitable for use with an apparatus, system, and/or method of the present disclosure, according to one embodiment. FIG. 8 is a dorsal perspective dorsal view of a foot 200. The bones of the forefoot and midfoot are illustrated. A first proximal phalange 230, second proximal phalange 232, third proximal phalange 234, fourth proximal phalange 236, and fifth proximal phalange 238 and a first distal phalange 240 are illustrated.

In the illustrated example, an arthrodesis surgical procedure, such as a Lapidus procedure has been performed on the TMT joint 802 (fixation hardware omitted). This may have been prescribed to correct for a hallux valgus condition of the foot 200. The Lapidus procedure on the TMT joint 802 has corrected the longitudinal axis 804 of the first metatarsal 208 such that the longitudinal axis 804 is substantially parallel with the second metatarsal 210.

However, this example foot 200 still has a deformity that presents near the distal one third portion of the big toe (first metatarsal 208, first proximal phalange 230, and first distal phalange 240). The first proximal phalange 230 and first distal phalange 240 extend laterally and/or dorsally at the metatarsophalangeal joint (MTP) 806. When a patient presents with a condition illustrated in FIG. 8, a surgeon or medical practitioner may advise a surgical procedure that straightens the first proximal phalange 230 and first distal phalange 240 in relation to the longitudinal axis 804 of the first metatarsal 208. The surgical procedure is designed to align the first proximal phalange 230 and first distal phalange 240 with the first metatarsal 208. Said another way, the surgical procedure brings a distal articular surface of the first metatarsal 208 (the articular surface of the distal head 808 into alignment with the longitudinal axis 804 of the first metatarsal 208.

FIG. 8 further illustrates the deformity by illustrating a relationship between the longitudinal axis 804 and an articular axis 810 extending from the distal articular surface of the first metatarsal 208. The articular axis 810 and longitudinal axis 804 intersect at an angle A. In the illustrated example, angle A may be about 20 degrees. This deformity can cause impingement for a patient and can lead to pain and discomfort, even though a successful Lapidus procedure has been performed straightening the first metatarsal 208.

Advantageously, another surgical procedure can be performed on the MTP 806 to remediate and/or correct the deformity. This surgical procedure is called a Reverdin procedure. The Reverdin procedure is a medializing closing wedge (i.e., a medializing closing wedge osteotomy) formed on a medial side of the distal end of the first metatarsal 208. Preferably, the osteotomies of the Reverdin procedure are positioned just proximal of the head 808. One goal of the Reverdin procedure may be to make the osteotomies in, at, or near, the transition from an epiphyseal region to a diaphyseal region of a distal end of the first metatarsal 208.

The Reverdin procedure involves a medial approach through the skin of the patient to form one or more osteotomies in the distal end of the first metatarsal 208 just proximal to the head 808. The Reverdin procedure resects a wedge of bone from the medial side that is angled and sized to provide the desired correction when the wedge osteotomy is closed. Furthermore, in certain embodiments, the Reverdin procedure may complete the wedge within the first metatarsal 208 and not cut through the lateral cortex of the bone. This forms a living hinge that facilitates reduction and management of the bone fragments created by the osteotomies.

In addition, improvements have been made to the Reverdin procedure which are variations of the original procedure and are named after those involved in the development of these revised procedures. These are the Reverdin-Green procedure, the Reverdin-Laird procedure, and the Reverdin-Todd procedure. Each of these variations adds an additional level of complexity but addresses a limitation discovered with using the original Reverdin procedure.

Conventionally, performing a Reverdin procedure and/or one of its variations (Reverdin-Green, Reverdin-Laird, and Reverdin-Todd) (all referred to herein as “Reverdin procedures” or “Reverdin procedure”) has been very challenging for a surgeon because the procedures include a plurality of osteotomies that need to be done with high precision, high accuracy, and at specific angles, on very small bones of a patient. Further, determining a location for and positioning the osteotomies and orienting the osteotomies of a Reverdin procedure can be very challenging for a surgeon because no osteotomy guides have been developed. Thus, a surgeon is left to execute the Reverdin procedures free-hand, without the aid of a guide.

Advantageously, the present disclosure provides a solution that overcomes the challenges of Reverdin procedures by providing a resection guide that is patient-specific and configured to register to the bone of the patient in a manner that matches a position of a model resection guide positioned preoperatively on a bone model of the bone that will receive the osteotomy during the surgical procedure.

Advantageously, using the apparatus, system, and/or methods of the present disclosure, a surgeon can pre-design, pre-plan, and prepare a resection guide that can guide one or more osteotomies for complex osteotomies such as the Reverdin procedures. The present disclosed embodiments may use a bone model created based on medical imaging of at least a portion of bone and/or one or more bones of a patient's foot. The bone model and/or the resulting resection guide resemble an anatomy of the patient's foot and/or the bone involved in the osteotomy. A surgeon can direct and/or prescribe the position of the osteotomy by providing instructions for where the resection guide will be placed on the bone (e.g., on the medial side of a neck between a shaft and the head 808 of the first metatarsal 208).

One or more bone engagement features can be added to the resection guide to facilitate desired positioning and/or orientation of the resection guide during the procedure. For example, in one embodiment, once positioned in relation to a bone model, a bone engagement surface can be formed on a side of the resection guide that will seat and/or register to the features and/or landmarks of a surface and/or other structure on the actual bone of the patient when the resection guide is positioned for the osteotomy. Once designed in a model resection guide, the model resection guide is fabricated for a particular patient for a specific procedure. FIG. 8 illustrates that at the osteotomy site 812 a medial surface of the first metatarsal 208 includes raised portions, lower portions, one or more concave sections, and the like which are created in a mirror image on a bone engagement surface formed on a side of a resection guide that will be placed on, or adjacent to, the bone for the osteotomy. These features of the bone surface and the corresponding features of the bone engagement surface enable the resection guide to seat, register, almost seek out or “find” its same corresponding location on the actual bone as the location determined and used with a bone model and model of the resection guide.

A surgeon can use the model of the bones of the patient, reference indicators such as axis of structures and/or bones of the foot and a model of the resection guide to determine or provide instructions for where one or more resection features (e.g., slots or channels) are to be placed, positioned, and/or oriented within the resection guide. The resection features guide a cutting tool to form one or more osteotomies in the bone. The resection features also define one or more trajectories for cuts into the bone. A depth guide on the cutting tool and/or a stop on the resection guide can control the depth of the osteotomy into the bone to facilitate retaining a cortex part of the bone. By accurately positioning a custom patient-specific resection guide, a surgeon can perform accurate and precise osteotomies (e.g., desired angles, straightness, lengths, depths, and the like) for a Reverdin procedure such that desired patient outcomes can be achieved.

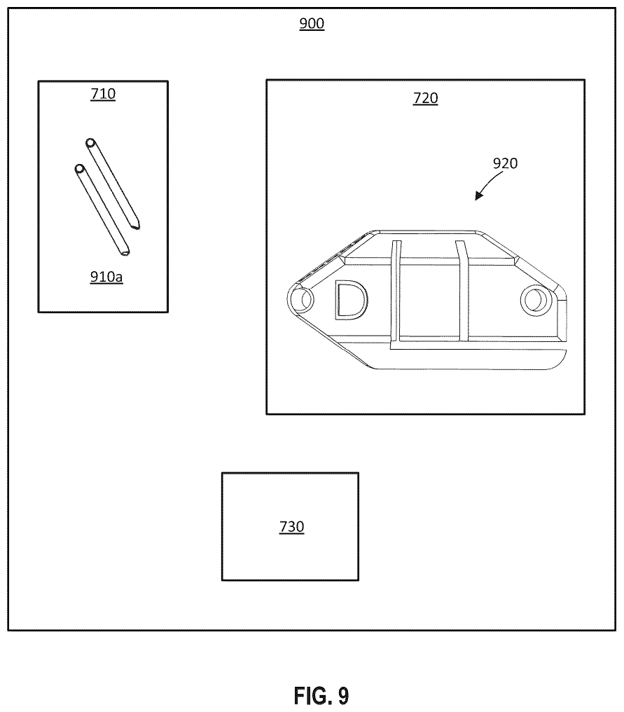

FIG. 9 illustrates an exemplary osteotomy system 900, according to one embodiment.

The osteotomy system 900 can include one or more fasteners 910a, one or more resection guides 920, and zero or one or more complementary components 730. The resection guide 920 may also include one or more resection features 922, one or more bone attachment features 924, one or more bone engagement feature 926, and/or one or more landmark registration feature 930. In certain embodiments, a bone engagement feature 926 can include a bone engagement surface 928.

While specific embodiments of complementary components 730 are not specifically shown here in relation to the osteotomy system 900, those of skill in the art will appreciate that complementary components 730 can be similar in feature, design, implementation, configuration, and purpose as those described in relation to the osteotomy system 700 and can be used for the osteotomy system 900. Thus, the osteotomy system 900 can include one or more alignment guides 740, rotation guides 750, correction guides 760, compression guides 770, positioning guides 780, fixation guides 790, navigation guides 792, implants 794, or the like.

The resection guide 920 may be a custom patient-specific resection guide made for a particular patient and/or for a particular surgical procedure. Various aspects of the resection guide 920 may be patient-specific, including, but not limited to, an angle and/or orientation for a resection feature of the resection guide 920, a position of the resection feature, a depth of the resection feature, a size of the resection guide 920, a configuration and/or composition of a bone contacting surface such as a bone engagement surface of the resection guide 920, a position of the resection guide 920 relative to one or more bones of a patient, and the like.

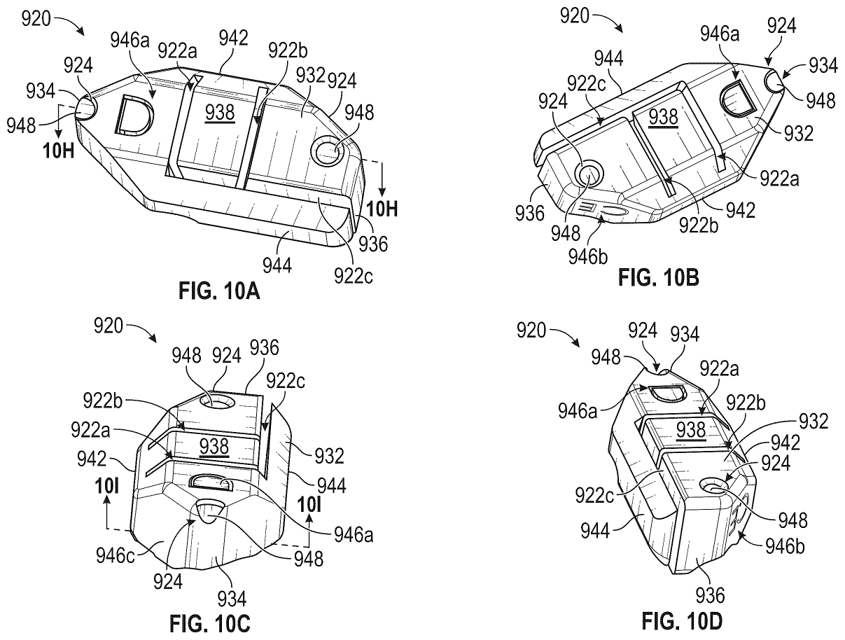

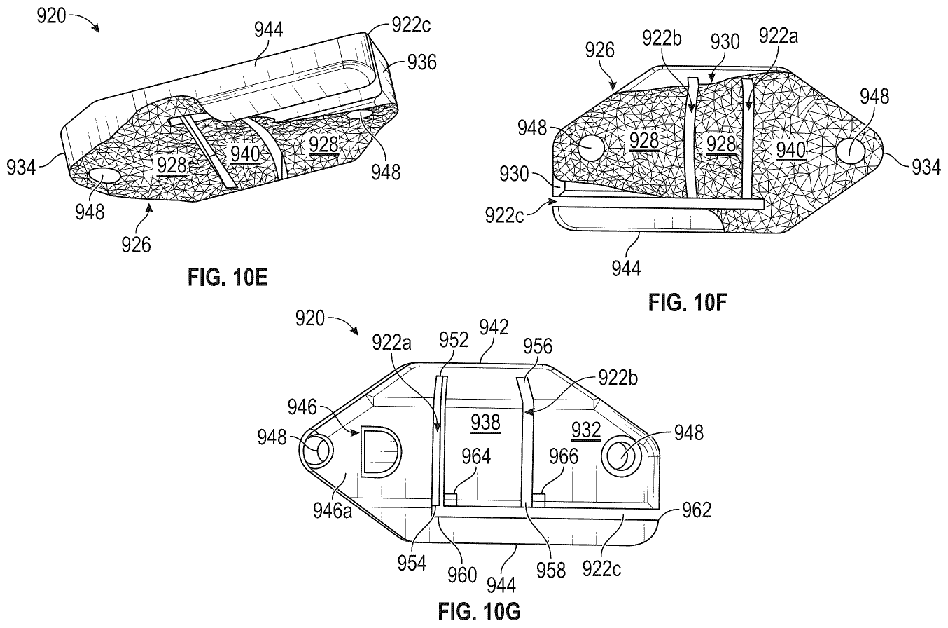

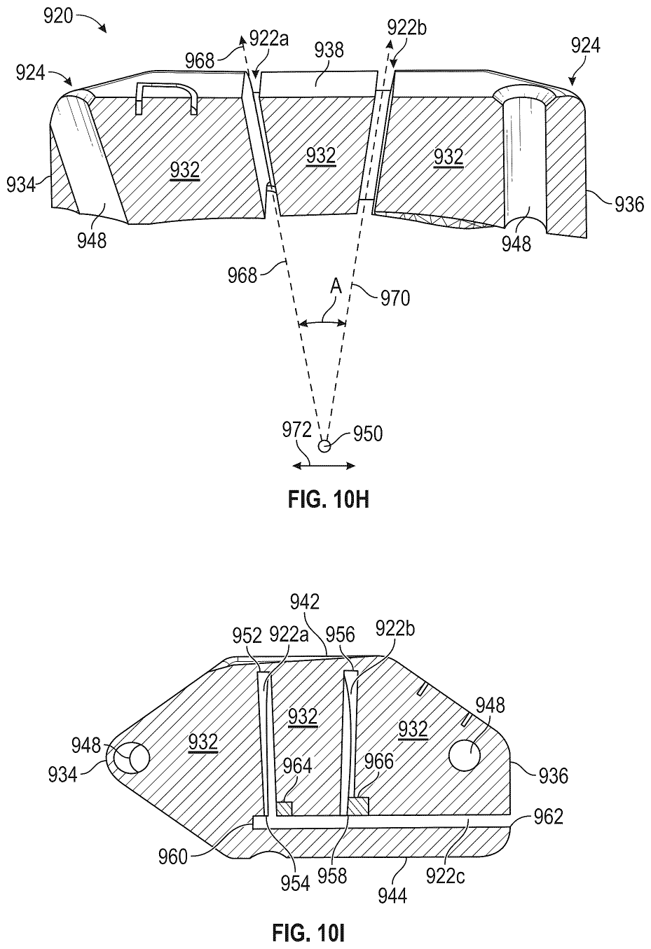

Referring to FIG. 9 and FIGS. 10A-10G, in one embodiment, the resection guide 920 includes a body 932 that includes an anterior side 934, a posterior side 936, a medial side 938, a lateral side 940, a superior side 942, and an inferior side 944. Generally, the sides of the resection guide 920 refer to the direction the sides face when the resection guide 920 is in use.

In certain embodiments, a position of one or more of the resection features 922 of the resection guide 920 can be determined and/or defined at least partially based on a bone model of at least a portion of a bone of a patient's foot. In one embodiment, the resection features 922 may be positioned to remove a minimal amount of bone and still implement a deformity correction.

In one embodiment, the position and/or configuration of the resection features 922 may be determined, defined, and/or dictated by both a bone model of a portion of a patient's foot and user instructions 604 (e.g., a surgeon's instructions). Advantageously, the user instructions 604 may identify certain goals for the resection feature 922 such as minimal size (i.e., width, length, depth), shape, contour, position, orientation, trajectory, and the like. Since the resection feature 922 guides the formation of an osteotomy, the configuration of the resection feature 922 provided by the user instructions 604 can also define an osteotomy created using the resection feature 922.

In certain embodiments, a user, or end user, may review the bone model, and a model of the resection guide 920 and based on these models determine, at least in part, where to position one or more of the resection features 922 within a resection guide 920. Advantageously, by reviewing and working with the models, a user and/or end user can minimize the amount of bone removed to perform a successful surgical procedure. Determining a position for the resection features 922 in a model of an instrument can be directly reflected in a fabricated instrument based on the model. In one embodiment, the position of a resection feature can be based on user instructions 604. Alternatively, or in addition, other features of a resection guide 920 can be based, at least partially, on user instructions 604. Alternatively, or in addition, in one embodiment, features of a resection guide 920 can be based on accepted practice aspects for a particular surgical procedure.

In one embodiment, the one or more resection features 922 are configured to guide a cutting tool to form one or more osteotomies in a bone of a patient. In certain embodiments, the resection guide 920 can include a first resection feature 922a, second resection feature 922b, and a third resection feature 922c. The first resection feature 922a is configured to form a first osteotomy having a first trajectory. The second resection feature 922b is configured to form a second osteotomy having a second trajectory. The third resection feature 922c is configured to form a third osteotomy having a third trajectory. The first osteotomy, second osteotomy, and third osteotomy may be configured to cooperate with each other to form a wedge osteotomy between bone fragments of a bone of the patient for a procedure such as a Reverdin procedure (e.g., Reverdin-Green).

FIGS. 10A-10G illustrate views of a resection guide 920 of the osteotomy system of FIG. 9, according to one embodiment. The resection guide 920 is configured for use to remediate a bone condition present in a patient's foot. In one embodiment, the resection guide 920 is patient-specific and is configured for use in a Reverdin procedure to correct a deformity near the MTP 806.

The resection guide 920 includes a body 932 having an anterior side 934, a posterior side 936, a medial side 938, a lateral side 940, a superior side 942, and an inferior side 944. The resection guide 920, in one embodiment, includes a first resection feature 922a, a second resection feature 922b, and a third resection feature 922c. The first resection feature 922a is configured to guide a cutting tool to form a first osteotomy in a bone, the first resection feature 922a extends through the resection guide 920 from the medial side 938 to the lateral side 940 along a first trajectory. The second resection feature 922b is configured to guide a cutting tool to form a second osteotomy in the bone, the second resection feature 922b extends through the resection guide 920 from the medial side 938 to the lateral side 940 along a second trajectory. The third resection feature 922c is configured to guide a cutting tool to form a third osteotomy in the bone, the third resection feature 922c extends through the resection guide 920 from the medial side 938 to the lateral side 940 along a third trajectory.

In certain embodiments, at least one of the first trajectory, the second trajectory, and the third trajectory are at least partially determined based on a bone model of at least a portion of the bone. The bone model is based, at least in part, on medical imaging of the bone, a portion of the bone, and/or a patient's foot and the bone model is configured to resemble an anatomy of the patient's foot. Since the first trajectory, the second trajectory, and the third trajectory relate directly to the first resection feature 922a, second resection feature 922b, and third resection feature 922c, the first resection feature 922a, second resection feature 922b, and third resection feature 922c are also at least partially determined based on the bone model of at least a portion of the bone.

The resection guide 920 also includes a bone attachment feature 924 configured to secure the resection guide 920 to the bone. The bone attachment feature(s) 924 may be similar to, or identical to, the bone attachment features 724 described herein. In particular, the bone attachment feature 924 may be embodied as a hole or opening 948 that cooperates with a fastener 910.

In certain embodiments, the resection guide 920 may also include one or more indicators 946. In the illustrated example, the resection guide 920 includes indicator 946a, indicator 946b, and indicator 946c. In the illustrated embodiment, indicator 946a is a debossed letter “D” that indicates to a user that this end of the resection guide 920 is the distal end and should be distal with respect to the user and/or patient during the procedure. Indicator 946b is a debossed set of letters which may indicate initials for a patient for whom the resection guide 920 will be used. Indicator 946c is a debossed set of letters and/or numbers that may indicate a serial number identifier for the resection guide 920.

FIG. 10A illustrates an inferior side perspective view of an example resection guide 920. FIG. 10B illustrates a superior side perspective view of the example resection guide 920. FIG. 10C illustrates an anterior side perspective view of the example resection guide 920. FIG. 10D illustrates a posterior side perspective view of the example resection guide 920.

FIGS. 10A-10D illustrate views of a resection guide 920 of the osteotomy system of FIG. 9, according to one embodiment. The resection guide 920 includes one or more resection features 922, one or more bone attachment features 924 (e.g., a fastener 910 and opening), and one or more bone engagement features 926 which may include one or more bone engagement surfaces 928.

Advantageously, the resection features 922 can be positioned, sized, and/or oriented to enable a surgeon to resect practically any shape in the bone for the osteotomy procedure. In the illustrated embodiment, the resection features 922 are configured to direct a cutting tool at an angle in one or more planes into the bone. The angle may be normal to a longitudinal axis of the bone, at an oblique angle, at an acute angle, or at an obtuse angle relative to a longitudinal axis of the bone. Advantageously, the size, shape, and angle of the resection can be predefined and can be determined preoperatively and/or can be patient-specific. In this manner, the resection guide 920 serves to provide for a patient-specific osteotomy procedure. Alternatively, or in addition, the resection features 922 can be configured to enable a surgeon to readily resect in a plantar direction and/or a dorsal direction. In certain embodiments, the resection features 922 may include an opening on one end or the other or both ends to permit the surgeon to position a cutting tool to make desired cuts that can extend laterally or medially.

FIGS. 10E and 10F illustrate a bone engagement feature 926 that can include a bone engagement surface 928 of the example resection guide 920. A bone engagement feature is any structure configured to engage or assist in the engagement of one or more bones. Thus, any structure that engages or assists in the engagement of a resection guides 920 with a bone is a bone engagement feature. In one embodiment, the position of the resection guide 920 on the bone (e.g., a distal end) may be determined by a surgeon preoperatively. Advantageously, the bone engagement feature 926 is configured to engage with at least a portion of the bone at a position that substantially matches a model position of a model of the resection guide engaging the bone model. The model of the resection guide and/or the bone model may be positioned by a technician and/or a surgeon or both working together. Once positioned, the resection guide 920 can be fabricated from the model of the resection guide. This fabrication includes formation of the bone engagement feature 926 and/or bone engagement surface 928.

In one embodiment, the bone engagement feature 926 includes a bone engagement surface 928 configured to at least partially match a contour of a surface of the bone receiving an osteotomy when the resection guide is positioned for use. The bone engagement surface 928 can be on any side of the resection guide 920. The side that includes the bone engagement surface 928 can depend on the approach used for the surgical procedure. In one embodiment, the bone engagement surface 928 is on the lateral side 940 of the body 932 in another embodiment, the bone engagement surface 928 may be on the medial side 938 of the body 932.

In the illustrated embodiment, the bone engagement surface 928 may include a portion of, or substantially all of, the surface of the lateral side 940 that contacts, or will contact, bone surface during a surgical procedure. In certain embodiments, the bone engagement surface 928 registers with a surface of the bone abutting the resection guide 920 when the resection guide is positioned for use (e.g., for the osteotomy).

The bone engagement surface 928 is configured to register to (i.e. seated or fit with) a surface of the bone, directly below and/or in contact with the body section when the resection guide 920 is in use. As one example, this means that projections from the surface of the bone (e.g., first metatarsal 208) fit within voids in the bone engagement surface 928 when the resection guide 920 is in use and in a desired position relative to a position of a model of the resection guide 920 in relation to a model of a patient's bone. The bone engagement surface 928 facilitates registration of the resection guide 920 to the bone (e.g., first metatarsal 208).

User instructions 604 may initiate the design and/or fabrication of the resection guide 920. For example, a surgeon may desire to have as much surface area on the lateral side 940 as possible while still keeping the overall size of the resection guide 920 as small as practicable. Accordingly, in the illustrated embodiment, a surgeon may have provided instructions for the length and/or width of the body 932. In this manner, a surgeon may maximize an amount of surface area of the resection guide 920 that contacts a bone to facilitate initial positioning intraoperatively to match or substantially match a position planned using a model of the resection guide 920 and a model of the bone. In certain embodiments, a surgeon may, at their discretion, include in the user instructions 604, instructions to add another body section that includes a bone engagement surface 928 on another side of the resection guide 920.

Referring now to FIG. 10F, in certain embodiments, the resection guide 920 can include a landmark registration feature 930. In the illustrated embodiment, at least one landmark registration feature 930 extends from the lateral side 940. Advantageously, the landmark registration feature 930 can provide a surgeon with confidence and assurance in the placement and positioning of the resection guide 920 on the bone because the landmark registration feature 930 can be configured to engage with a particular landmark on the bone (e.g., a side surface of the bone (dorsal, plantar, medial, lateral, a projection or a depression or cavity, or the like). Alternatively, or in addition, the landmark registration feature 930 can include a contoured bone engagement surface 928 that can further facilitate registration of the landmark registration feature 930 and/or resection guide 920 with the bone. In this manner, a surgeon can be assured intraoperatively that the resection guide 920 is being positioned as desired.

In certain embodiments, the landmark registration feature 930 can be shaped like a hook to engage a surface or structure of a bone. Alternatively, or in addition, the resection guide 920 may include a landmark registration feature 930 on each side (superior and inferior), together the landmark registration features 930 can engage one or more landmarks of a surface of the bone such that the surgeon can accurately position and register the resection guide 920 to the bone.

FIG. 10G illustrates a medial side 938 perspective view of the resection guide 920. In the illustrated embodiment, the first resection feature 922a is parallel to the second resection feature 922b at the surface of the medial side 938 and the two resection features 922 are angled towards each other as they extend through the body 932 (See FIG. 10H). In the illustrated embodiment, the first resection feature 922a and second resection feature 922b are configured to converge and intersect with each other such that a first osteotomy formed using the first resection feature 922a follows a first trajectory and second osteotomy formed using the second resection feature 922b follows a second trajectory and the two trajectories intersect at a vertex 950 (See FIG. 10H).

The first resection feature 922a includes a superior end 952 and an inferior end 954. The second resection feature 922b includes a superior end 956 and an inferior end 958. The third resection feature 922c includes a distal end 960 and a proximal end 962. Those of skill in the art will appreciate that the customized and/or patient-specific nature of how the resection guide 920 is designed and fabricated mean that the position, number, orientation, and/or relationship among the resection features 922 can vary between embodiments and/or users and/or surgeons and/or procedures.

In one embodiment, the third resection feature 922c intersects at least one of the first resection feature 922a and the second resection feature 922b. In the illustrated embodiment, the third resection feature 922c intersects both the first resection feature 922a and the second resection feature 922b. In addition, the first resection feature 922a includes a superior end 952 that is closed meaning that a cutting tool operating within the first resection feature 922a cannot proceed past the superior end 952. The first resection feature 922a includes an inferior end 954 that is closed in the sense that a cutting tool moving within the first resection feature 922a is stopped by the intersecting third resection feature 922c. The inferior end 954 is open in the sense that if the cutting tool (e.g., a burr) can navigate from the first resection feature 922a to the third resection feature 922c, the cutting tool can proceed past the inferior end 954.

In addition, the second resection feature 922b includes a superior end 956 that is closed meaning that a cutting tool operating within the second resection feature 922b cannot proceed past the superior end 956. The second resection feature 922b includes an inferior end 958 that is closed in the sense that a cutting tool moving within the second resection feature 922b is stopped by the intersecting third resection feature 922c. The inferior end 958 is open in the sense that if the cutting tool (e.g., a burr) can navigate from the second resection feature 922b to the third resection feature 922c the cutting tool can proceed past the inferior end 958.

The third resection feature 922c includes a distal end 960 that is a closed end meaning that a cutting tool operating within the third resection feature 922c cannot proceed past the distal end 960. The third resection feature 922c also includes a proximal end 962 that is an open end meaning that a cutting tool operating within the third resection feature 922c can proceed past the proximal end 962. Advantageously, the closed end, the distal end 960, may assist a surgeon by helping the surgeon to not resect more distal than the distal end 960 to preserve and/or retain certain structures (e.g., a set of sesamoids). The open end, proximal end 962, may enable a surgeon to resect more proximal if needed and determined by the surgeon during a procedure. Those of skill in the art will appreciate that the proximal end 962 can also be a closed end in certain embodiments.

In certain embodiments, the third resection feature 922c may intersect with the first resection feature 922a and/or the second resection feature 922b to facilitate forming osteotomies for a particular procedure (e.g., Reverdin-green). The first resection feature 922a and second resection feature 922b enable a surgeon to form a wedge osteotomy that starts from a medial side of the bone. The third resection feature 922c enables a surgeon to implement a Reverdin-green osteotomy parallel to a plantar weight bearing surface of the bone (e.g., horizontal cut), proximal to a distal articular surface of the bone and superior to a plantar aspect of a head of the bone. In one embodiment, the third resection feature 922c enables a surgeon to implement a Reverdin-green osteotomy parallel to a plantar weight bearing surface of a first metatarsal 208, proximal to a distal articular surface of the first metatarsal 208 and superior to a plantar aspect of the first metatarsal 208.

FIG. 10G illustrates that the third resection feature 922c intersects the inferior end 954 of the first resection feature 922a at a first intersection angle 964 and intersects the second resection feature 922b at a second intersection angle 966. An intersection angle is an angle between two intersecting resection features 922. In the illustrated embodiment, the first intersection angle 964 and the second intersection angle 966 may be equal and may be about 90 degrees. Advantageously, the third resection feature 922c intersecting inferior end 954 and inferior end 958 may facilitate a surgeon in forming a plantar shelf during the surgical procedure.

Those of skill in the art will appreciate that the relationship between the resection features 922 and any intersection angles between them can be determined to address patient-specific needs and/or surgeon preferences or instructions. Accordingly, in certain embodiments, the resection features 922 can be arranged to facilitate completing a Reverdin-Laird or Reverdin-Todd procedure. In a Reverdin-Todd procedure, the intersection angles (e.g., first intersection angle 964 and/or second intersection angle 966) can be greater than or less than 90 degrees. In one embodiment, at least one intersection angle between the resection feature 922c and one of the first resection feature 922a and the second resection feature 922b is between about 45 degrees and about 135 degrees.

FIG. 10H illustrates a cross-section view of the resection guide of FIG. 10A taken along line 10H, according to one embodiment. FIG. 10G illustrates angles for bone attachment features 924 (e.g., fasteners 910 in openings). In the illustrated embodiment, the angles cause the bone attachment features 924 (e.g., fasteners 910) to extend into the bone at an angle. Advantageously, a surgeon may predefine an angle for one or more bone attachment features 924 preoperatively.

In certain embodiments, the openings 948, opening positions, and/or angles for the openings 948 may be determined with the resection guide 920 to facilitate subsequent fixation for a surgical procedure. For example, the size, position, and/or angle of the openings 948 may be determined such that once the fasteners 910 are removed and the resection guide 920 is removed, the holes formed by the fasteners 910 can be used for a fixation fastener such as a bone staple. Alternatively, or in addition, angling one or more of the openings 948 enables the deployed fasteners 910 in the openings 948 to securely hold and/or retain the resection guide 920 in place during one or more osteotomies.

In one embodiment, the bone attachment feature 924 is configured to form at least one hole in the bone of the patient. For example, the bone attachment features 924 may include an opening 948 configured to accept a fastener 910 (i.e., a K-wire). The bone attachment feature 924, opening 948, and/or fastener 910 may be configured, oriented, and/or angled relative to the bone and/or bone fragments after an osteotomy to facilitate use of a fixation device. In one embodiment, the bone attachment feature 924 is configured such that a hole created when a fastener 910 is removed from an opening 948 of the bone attachment feature 924 forms at least one hole. This hole may serve as an anchor hole for a fixation device (i.e., a bone staple). In this manner, use of the resection guide 920 enables reuse of holes formed on bone fragments to facilitate subsequent fixation using one or more fixation devices.

FIG. 10H also illustrates an angle for the first resection feature 922a and second resection feature 922b through the body 932 of the resection guide 920. An opening of the first resection feature 922a and/or the opening of the second resection feature 922b has a width and/or length large enough to accommodate, or accept, a cutting element of a cutting tool. In one embodiment, the opening of the first resection feature 922a is configured to guide and/or enable a cutting tool to form a first osteotomy into, and/or through, the bone. Advantageously, the first osteotomy tracks, follows, and/or is aligned with a first trajectory 968.

In one embodiment, an opening of the second resection feature 922b is configured to guide and/or enable a cutting tool to form a second osteotomy into, and/or through, the bone. Advantageously, the second osteotomy tracks, follows, and/or is aligned with the second trajectory 970. Thus, a surgeon operating the cutting tool within the opening of the first resection feature 922a and the second resection feature 922b can readily form a first osteotomy and a second osteotomy that matches a design that may have been set out in a model of the resection guide 920 and/or a model of the patient's bone(s).

In one embodiment, the first trajectory 968 may be determined and/or defined to be a patient-specific feature. Similarly, the second trajectory 970 may be determined and/or defined to be a patient-specific feature. Advantageously, using the apparatus, methods, and/or systems of the present disclosure a user may determine and/or at least partially determine the first trajectory 968 and/or the second trajectory 970 based on a bone model of at least a portion a bone of the patient that is to receive one or more osteotomies. The bone model of at least a portion of the bone can be derived from and/or based on medical imaging of a patient's foot. In certain embodiments, the bone model used to determine, or at least partially determine, the first trajectory 968 and/or the second trajectory 970 is configured to resemble, substantially resemble, or match the anatomy of the patient's foot.

As used herein, in certain embodiments, partial determination of the first trajectory 968 and/or the second trajectory 970 based on a bone model may mean that the bone model for the a bone of a patient that will receive the osteotomies is used together with other imaging data, anatomic data, patient imaging data, patient data, information from a prescription from a doctor, information about surgeon preferences, measurement data taken from the bone model or medical imaging, or the like may also be used to determine the first trajectory 968 and/or the second trajectory 970.

In one embodiment, the first resection feature 922a extends through the resection guide 920 from the medial side 938 to the lateral side 940 along the first trajectory 968. The first trajectory 968 is at least partially determined based on a bone model of at least a portion of a bone of a patient's foot. The bone model is based on medical imaging of the patient's foot and is configured to resemble, significantly resemble, and/or match the anatomy of the patient's foot.

Alternatively, or in addition, the second resection feature 922b extends through the resection guide 920 from the medial side 938 to the lateral side 940 along the second trajectory 970. The second trajectory 970 is at least partially determined based on a bone model of at least a portion of a bone of a patient's foot. The bone model is based on medical imaging of the patient's foot and is configured to resemble, significantly resemble, and/or match the anatomy of the patient's foot.

Those of skill in the art will appreciate that the first trajectory 968 and the second trajectory 970 can define a path for the osteotomies formed using the first resection feature 922a and/or second resection feature 922b. Alternatively, or in addition, where a surgical procedure plans to perform a wedge osteotomy, the first trajectory 968 and the second trajectory 970 can predefine the size, shape, and configuration of a wedge fragment formed by the osteotomy.

In one embodiment, a surgeon may desire to perform a wedge osteotomy. (e.g., Reverdin procedure) In this embodiment, the resection guide 920 is designed such that the first trajectory 968 converges with the second trajectory 970 at a vertex 950 having a wedge angle A, such that first resection feature 922a and the second resection feature 922b form a wedge osteotomy comprising a wedge bone fragment (not shown). The wedge angle A can be determined based, at least in part, on the bone model. Advantageously, the various aspects of the design of the resection guide 920 and/or an accompanying surgical technique and/or complementary components 730 can be done prior to fabrication of one or more components of the osteotomy system 900. Thus, a surgeon can define or adjust the position of the vertex 950, the number of degrees for the wedge angle A, a height of the body 932, and the like. Of course, certain of these aspects may be predefined for a surgeon and/or recommendations made to a surgeon. Alternatively, or in addition, certain of these aspects may be patient-specific while others may be standard based on experience and/or established practice for a particular procedure.

In certain embodiments, a surgeon and/or a technician working with the surgeon, may determine the size, shape, and/or configuration of a wedge bone fragment to be removed from the bone. Furthermore, a surgeon and/or a technician can determine whether to perform an osteotomy that forms a wedge bone fragment or an osteotomy that enables an opening wedge osteotomy or an osteotomy that does not include a wedge (closing or opening). The type of osteotomy performed can determine whether or not the first trajectory 968 and/or second trajectory 970 converge.

Those of skill in the art will appreciate that the first trajectory 968 and/or the second trajectory 970 may extend from the medial side 938 to the lateral side 940 at an angle of between about 175 degrees to an angle of about 10 degrees. Thus, in certain embodiments, the first trajectory 968 and the second trajectory 970 may converge and in other embodiments the first trajectory 968 and the second trajectory 970 may diverge. In certain embodiments, one or both of the first trajectory 968 and the second trajectory 970 may extend from the medial side 938 to the lateral side 940 at an angle of about 90 degrees.

In one embodiment, a surgeon can also determine, preoperatively, whether the vertex 950 will be inside the bone or outside the bone. For example, the surgeon may decide to have the vertex 950 outside the bone a distance away from a lateral surface of the bone (e.g., first metatarsal 208 for a Reverdin-Laird). The position of the vertex 950 may depend on the surgical procedure, surgeon preference, a surgeon's planned correction, or the like. In such an embodiment, the first trajectory 968 may converge with the second trajectory 970 at a vertex 950 outside the bone and spaced a distance from the lateral cortex 972 (represented by the line in FIG. 10H) of the bone.

In another instance, a surgeon may want the vertex 950 prepositioned between the lateral cortex 972 of the bone and the resection guide 920 when the resection guide 920 is designed for use for a surgical procedure (e.g., Reverdin-Green). In certain osteotomies, a surgeon may desire that the vertex 950 is positioned within the bone and offset from the lateral cortex 972 by a predetermined offset. Advantageously, using embodiments of the present disclosure a surgeon can determine and/or adjust the size of the predetermined offset.

A surgeon may desire to position the vertex 950 between the lateral cortex 972 of the bone and the resection guide 920 when the resection guide 920 is in use for a surgical procedure such that a first osteotomy, second osteotomy, and a third osteotomy leave bone between the vertex 950 and the lateral cortex 972 intact. This intact bone may serve to keep a distal bone fragment connected to proximal bone fragment. Depending on the surgical procedure planned, a surgeon may desire to keep this bone intact to serve as a “living hinge” which can be used to close the wedge osteotomy. Of course, different surgeons may have different sizes they want for predetermined offset (thickness of the lateral cortex 972). Alternatively, or in addition, the size of the predetermined offset may be based at least in part on how the patient presents for the procedure or preparation and planning for the procedure.

Advantageously, the present disclosure enables the thickness/size of the predetermined offset for the thickness of the lateral cortex 972 to be predetermined, to be patient-specific, as well as the determination of whether or not to have a predetermined offset. As described, a surgeon may preposition the vertex 950 to be within the bone or outside the bone.

In certain embodiments, the position of the vertex 950 may be customized to a particular patient. The vertex 950 position may be patient-specific. Alternatively, or in addition, the position of the vertex 950 may be set at a default predetermined offset, such as one millimeter. In another embodiment, the position of the vertex 950 may be predetermined, for example due to the type of osteotomy being performed.

In one embodiment, the position of the vertex 950 may be fixed due to the type of osteotomy to be performed. In another embodiment, the vertex 950 is prepositioned so that an osteotomy formed using two of the resection features forms a living hinge at a cortex of the bone. The living hinge can help the surgeon complete a surgical procedure by keeping two bone fragments connected. The living hinge connection can be permanent, and the hinge may be bent as the wedge opening is closed for fixation. Alternatively, or in addition, a the living hinge may be temporary, and a surgeon may break the living hinge when they are ready for a subsequent part of the surgical procedure (e.g., bone fusion, repositioning, rearrangement, fixation, or the like).

Those of skill in the art will appreciate that with a given position of the vertex 950 and based on a thickness of the bone at the location of the osteotomy, the size of the wedge angle A can directly impact the width of the body 932. Those of skill in the art will appreciate that the size of the wedge angle A can vary depending on the needs of the patient, surgeon preferences, anatomical data, or the like.

In one embodiment, the wedge angle A may range from between about 5.0 degrees to about 45.0 degrees. In one embodiment, the wedge angle A is about 13.4 degrees. In another embodiment, the wedge angle A is about 18.3 degrees. Advantageously, a user or surgeon can define and/or adjust the wedge angle A in a tool that views and/or edits parameters for the bone model and/or for a model of the resection guide 920.

The first trajectory 968 and second trajectory 970 of FIG. 10H illustrates how the wedge osteotomy will extend medial to lateral within the bone. The third resection feature 922c determines where a wedge fragment created using the first resection feature 922a and second resection feature 922b will stop in the dorsal to plantar direction. In the illustrated embodiment, the third resection feature 922c intersects the first trajectory 968 and the second trajectory 970 to for a wedge osteotomy.

FIG. 10I illustrates a cross-section view of the resection guide in FIG. 10C taken along line 10I of FIG. 10C, according to one embodiment. FIG. 10I illustrates a relationship between a first resection feature 922a, a second resection feature 922b, and a third resection feature 922c within the body 932.

The superior end 952 and inferior end 954 of the first resection feature 922a are illustrated. The superior end 956 and inferior end 958 of the second resection feature 922b are illustrated. And the distal end 960 and proximal end 962 of the third resection feature 922c are illustrated. Note that in the illustrated embodiment, the first intersection angle 964 and second intersection angle 966 are about 90 degrees. Orienting the third resection feature 922c to intersect the first resection feature 922a and second resection feature 922b at about 90 degrees facilitates forming a plantar shelf as part of a Reverdin-Green surgical procedure. The plantar shelf can be advantageous because this shelf can help prevent a distal bone fragment of the bone from rotating during the procedure and/or during reduction and/or fixation.

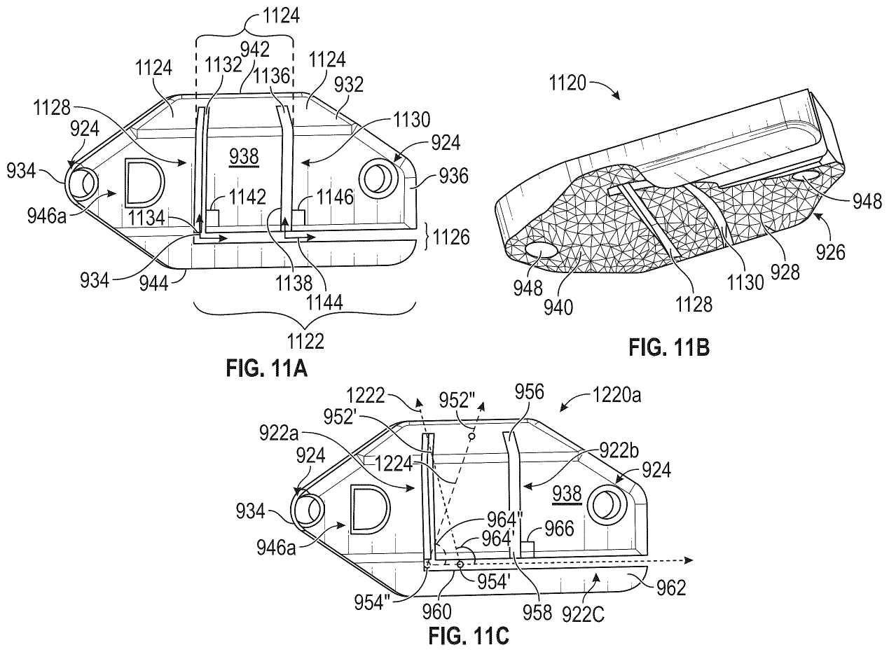

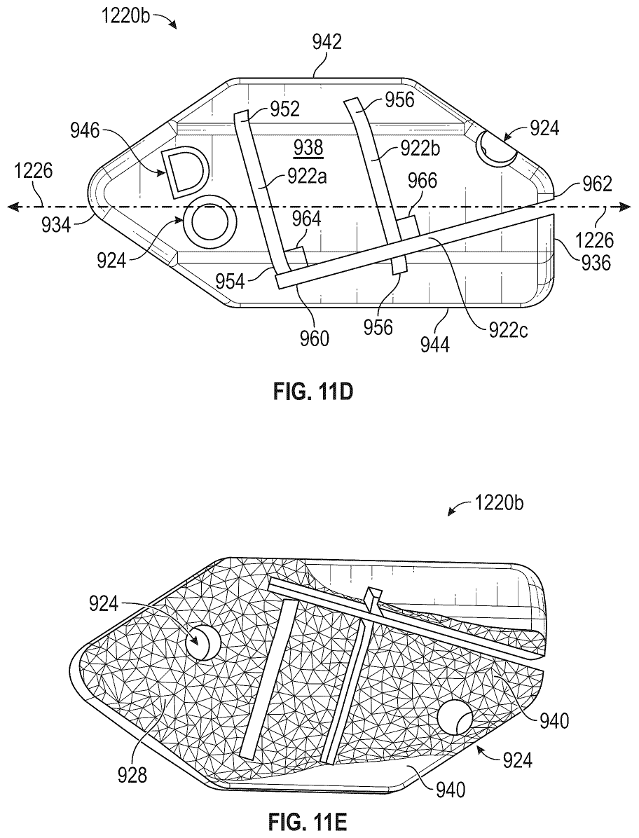

FIGS. 11A-11E illustrate views of resection guides of an osteotomy system, according to alternative embodiments. FIG. 11A illustrates an alternative embodiment of a resection guide 920. The osteotomy system includes resection guide 1120.

The resection guide 1120 of the osteotomy system can include some or all of the same or substantially the same features, aspects, and/or components as the resection guides (e.g., resection guide 920) described herein with like components including the same reference numerals. Accordingly, the resection guide 1120 can, or may include, one or more resection features 922, one or more bone attachment features 924, one or more bone engagement features 926, and the like. The one or more bone attachment features 924 are configured to secure the resection guide 1120 to a metatarsal of a patient. The resection guide 1120 may include a body 932 having an anterior side 934, posterior side 936, medial side 938, lateral side 940, superior side 942, and inferior side 944.

The resection guide 1120 may differ from other resection guides 920 described herein because the resection guide 1120 may include a single resection feature 1122. The resection feature 1122 is configured to guide a cutting tool to form a wedge osteotomy in a metatarsal (e.g., first metatarsal 208). The wedge osteotomy may also form a wedge bone fragment and a shelf (aka a plantar shelf) near a distal end of the metatarsal. The wedge osteotomy is at least partially determined based on a bone model of at least a portion of the metatarsal. In one embodiment, the metatarsal is the metatarsal of a patient for the wedge osteotomy.

In the illustrated embodiment, the resection feature 1122 may include a first section 1124 and a second section 1126. The first section 1124 extends through the resection guide 1120 from the medial side 938 to the lateral side 940. The first section 1124 includes a first leg 1128 extending through the resection guide 1120 along a first trajectory. The first section 1124 also includes a second leg 1130 that extends through the resection guide 1120 along a second trajectory.

The first leg 1128 may include a superior end 1132 and an inferior end 1134. The second leg 1130 may include a superior end 1136 and an inferior end 1138. The second section 1126 extends through the resection guide 1120 from the medial side 938 to the lateral side 940. The second section 1126 intersects the first leg 1128 at a first turn 1140 having a first intersection angle 1142 and the second leg 1130 at a second turn 1144 having a second intersection angle 1146. The second section 1126 extends through the resection guide 1120 along a third trajectory.

In the illustrated embodiment, the first intersection angle 1142 and the second intersection angle 1146 are about 90 degrees. Those of skill in the art will appreciate that the first intersection angle 1142 and the second intersection angle second intersection angle 1146 can be other angles such as 45 degrees, 20 degrees, 10 degrees, 135 degrees, and the like. In the illustrated embodiment, 90 degrees may be desired for performing a Reverdin-Green surgical procedure.

In the illustrated embodiment, the first leg 1128 and second leg 1130 can cooperate to guide a surgeon in forming a first osteotomy and second osteotomy that meet at a vertex 950 within the bone to form a wedge bone fragment that is wider on the medial side than the lateral side. The second section 1126 cooperates with first leg 1128 and the second leg 1130 to resect bone along a dorsal to plantar axis to form a straight through cut for a wedge osteotomy of a Reverdin-Green procedure.

In the illustrated embodiment, the wedge osteotomy includes an osteotomy formed using the second section 1126. The osteotomy is parallel to a plantar weight bearing surface of a first metatarsal 208, proximal to a distal articular surface of a head 808 and superior to a plantar aspect of a head 808 of the first metatarsal 208. Advantageously, the second section 1126 is positioned within the resection guide 1120 such that the second section 1126 protects sesamoids that are plantar to a head of the first metatarsal 208. The second section 1126 protects the sesamoids from dissection.

In one embodiment, the second section 1126 enables a surgeon to make an osteotomy that is parallel to a plantar weight bearing surface (i.e., a plantar surface of the first metatarsal 208). The osteotomy is also proximal to a distal articular surface and superior to a plantar aspect of the head of the first metatarsal 208. This osteotomy may be very challenging for a surgeon to make accurately and on such a small bone without the aid of the resection guide 1120. The second section 1126 helps prevent dissection of the sesamoids from the foot.

In certain embodiments, the resection feature 1122 is configured to form a shelf in a distal bone fragment of the first metatarsal 208 of a patient (on a distal side of a wedge osteotomy). The shelf may be formed after the osteotomy is completed or after one osteotomy of an osteotomy procedure is completed. The osteotomy procedure may include creating osteotomies using the first section 1124 and the second section 1126. In one embodiment, the resection guide 1220 is positioned on a lateral side at, near, or on a head of the first metatarsal 208. Using a cutting tool, an osteotomy may be formed using the first section 1124 and the second section 1126. After the osteotomies, a wedge osteotomy may be formed.

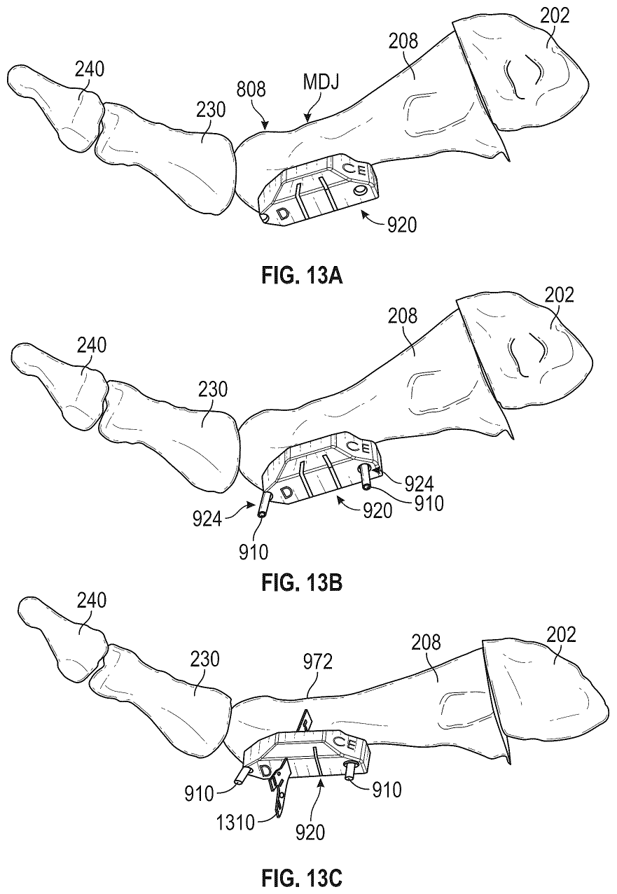

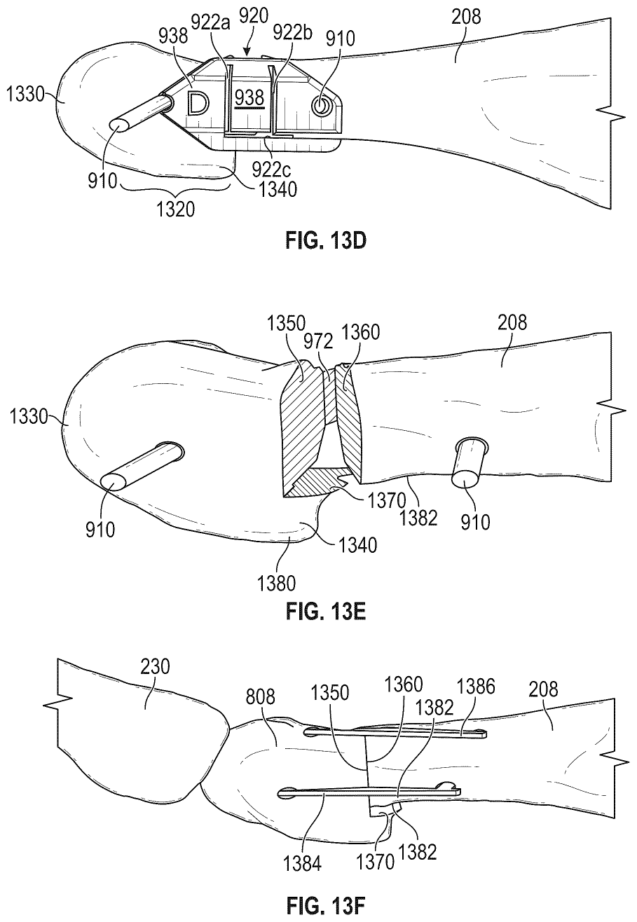

In one embodiment, the shelf may be formed after forming an osteotomy using the second section 1126. The shelf may be a planar surface that extends distally into the head of the first metatarsal 208 superior to the sesamoids. Alternatively, or in addition, forming an osteotomy using the second section 1126 may also form a planar surface on a plantar (inferior) side of a proximal bone fragment formed by osteotomies using the resection guide 1120. In one embodiment, the shelf may be defined by a section of the second section 1126 between the first leg 1128 and the second leg 1130. The planar surface on the plantar side of a proximal bone fragment may be defined by a section of the second section 1126 between the second leg 1130 and the posterior side 936 of the resection guide 1120. See FIG. 13E, plantar surface 1382 for an example planar surface.

In certain embodiments, the planar surface is on a plantar side of a proximal side of a wedge osteotomy. Alternatively, or in addition, planar surface may be on a dorsal side, a medial side, or a lateral side of a proximal bone fragment of a bone. In such embodiments, a shelf may be formed on a side of the head of the first metatarsal 208, the side corresponding to the planar surface. Advantageously, the planar surface and shelf are configured to engage with each other during fusion of the wedge osteotomy. This engagement provides increased bone contact for fusion of the wedge osteotomy.

FIG. 11B illustrates the resection guide 1120 from a lateral side perspective and shows the lateral side 940 and bone engagement surface 928. In the illustrated embodiment, the bone engagement surface 928 is on a lateral side 940 of the body 932 of the resection guide 1120.

The bone engagement surface 928 facilitates registration of the resection guide 1120 with a distal medial surface of a first metatarsal 208. In certain embodiments, the bone engagement surface 928 is configured based on a bone model of a bone of patient. The bone model includes a surface that will contact the resection guide 1120 when the surgical procedure is performed. The bone engagement surface 928 is configured to at least partially match a contour of a surface of the metatarsal 208 when the resection guide 1120 is positioned for use.

Advantageously, the bone model includes a contour of a surface of a metatarsal that is to be cut using the resection guide 1120. The contoured surface of the bone model is used to form a corresponding, mirrored, or inverse contour in a bone engagement surface 928 of the resection guide 1120, as described herein. The matching contour or at least partially matching contour of the bone engagement surface 928 and a contour of a surface (i.e., medial surface) of the first metatarsal 208 enables a surgeon to place or position the resection guide 1120 in the same position as planned in a preoperative plan.

FIG. 11C illustrates an alternative embodiment of a resection guide 920. The osteotomy system includes resection guide 1220a. The resection guide 1220a of the osteotomy system can include some or all of the same or substantially the same features, aspects, and/or components as the resection guides (e.g., resection guide 920, resection guide 1120, etc.) described herein with like components including the same reference numerals. Accordingly, the resection guide 1220a can, or may, include one or more resection features 922a, 922b, 922c, one or more bone attachment features 924, one or more bone engagement features 926, and the like. The resection guide 1220a may include a body 932 having an anterior side 934, posterior side 936, medial side 938, lateral side 940, superior side 942, and inferior side 944.

The resection guide 1220a may differ from other resection guides 920 described herein because the ends of the first resection feature 922a and second resection feature 922b may be configured such that the first resection feature 922a and the second resection feature 922b are not parallel at the surface of the medial side 938. Instead, the first resection feature 922a may have one of two alternative configurations with respect to the second resection feature 922b.

In a first configuration 1222, the inferior end 954′ of the first resection feature 922a may be closer to the inferior end 958 of the second resection feature 922b than the superior end 952′ is to the superior end 956. In this first configuration 1222, the first intersection angle 964′ may be greater than 90 degrees. In a second configuration 1224, the superior end 952″ of the first resection feature 922a may be closer the superior end 956 of the second resection feature 922b than the inferior end 954″ is to the inferior end 958. In this second configuration 1224, the first intersection angle 964″ may be less than 90 degrees.

Those of skill in the art will appreciate that the relationship between the first resection feature 922a, second resection feature 922b, and third resection feature 922c can impact the configuration of the closing osteotomy once all cuts are made and reduction is performed. Said another way, the relationship between the first resection feature 922a and the resection feature 922b can be used to define a shape for the wedge osteotomy formed using the resection guide 1220.

FIG. 11D illustrates an alternative embodiment of a resection guide 920. The osteotomy system includes resection guide 1220b. The resection guide 1220b of the osteotomy system can include some or all of the same or substantially the same features, aspects, and/or components as the resection guides (e.g., resection guide 920) described herein with like components including the same reference numerals. Accordingly, the resection guide 1220b can or may include one or more resection features 922a, 922b, 922c, one or more bone attachment features 924, one or more bone engagement features 926, one or more bone engagement surfaces 928, and the like. The resection guide 1220b may include a body 932 having an anterior side 934, posterior side 936, medial side 938, lateral side 940, superior side 942, and inferior side 944.

The resection guide 1220b may differ from other resection guides 920 described herein because the ends of the first resection feature 922a and second resection feature 922b may be configured such that the first resection feature 922a and the second resection feature 922b are parallel at the surface of the medial side 938 and the third resection feature 922c may extend along the body 932 at an angle that is not parallel to a long axis 1226 of the resection guide 1220.

The superior end 952 of the first resection feature 922a and the superior end 956 of the second resection feature 922b may be about the same distance apart from each other as the inferior end 954 of the first resection feature 922a and the superior end 956 of the second resection feature 922b. The first resection feature 922a and the second resection feature 922b may be angled at an angle that is not 90 degrees in relation to the long axis 1226. The third resection feature 922c may be angled within the body 932 at an angle that is not parallel to the long axis 1226. In the illustrated embodiment, the third resection feature 922c may intersect the first resection feature 922a at a first intersection angle 964 that is about 90 degrees and the second resection feature 922b may intersect the second resection feature 922b at a second intersection angle 966 that is about 90 degrees. In certain embodiments, a user may want to use the design of the resection guide 1220b for a particular surgical procedure (e.g., a Reverdin-Todd). In one embodiment, the resection guide 1220b may enable an osteotomy that forms a closing wedge and plantarflexes a head 808 of a first metatarsal 208 to correct for a head 808 that extends too far dorsally. The example embodiment may be referred to as a plantar flexion Reverdin guide.

Those of skill in the art will appreciate that while the third resection feature 922c can be directed dorsally to enable a surgical procedure that plantar flexes a distal end of a bone, embodiments of the present disclosure can also be used to fabricate a resection guide 1220 where the third resection feature 922c is directed plantarly to enable a surgical procedure that dorsiflexes a distal end of a bone. Such an embodiment may be referred to as a dorsiflexed Reverdin guide. Those of skill in the art will also appreciate that angles for the first resection feature 922a, second resection feature 922b, and/or third resection feature 922c relative to the long axis 1226 and/or to each other can be configured based on patient anatomy, a particular surgical procedure, surgeon preference, or the like.

FIG. 11E illustrates the resection guide 1120b from a lateral side perspective and shows the lateral side 940 and bone engagement surface(s) 928. The bone engagement surface 928 facilitates registration of the resection guide 1120b with a distal medial surface of a first metatarsal 208.

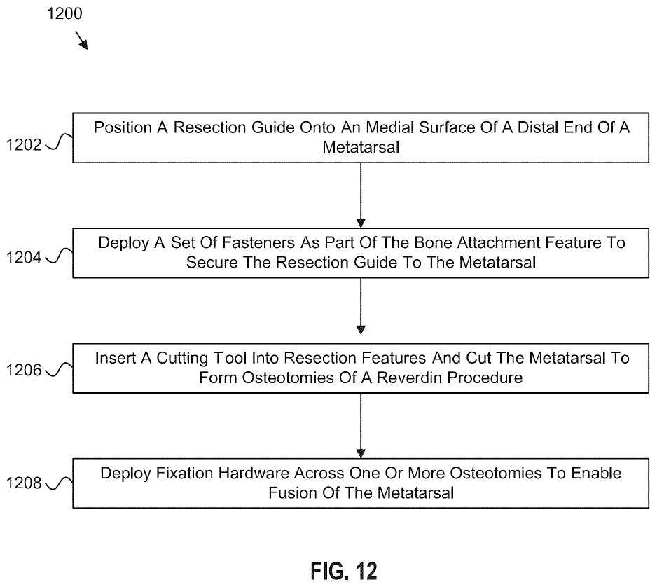

FIG. 12 is a flowchart of an example method 1200 or process 1200 for remediating a bone condition, according to one embodiment. In some implementations, one or more process blocks of FIG. 12 may be performed by an apparatus, device or system. As shown in FIG. 12, process 1200 may include positioning a resection guide onto a medial surface of a distal end of a metatarsal, the resection guide having: a body having an anterior side, a posterior side, a medial side, a lateral side, a superior side, and an inferior side.

The resection guide includes a first resection feature configured to guide a cutting tool to form a first osteotomy in a metatarsal, the first resection feature extending through the resection guide from the medial side to the lateral side along a first trajectory at least partially determined based on a bone model of at least a portion of the metatarsal, the bone model based on medical imaging of the patient's foot and configured to resemble an anatomy of the patient's foot.

The resection guide also includes a second resection feature configured to guide a cutting tool to form a second osteotomy in the metatarsal, the second resection feature extending through the resection guide from the medial side to the lateral side along a second trajectory at least partially determined based on the bone model.

The resection guide also includes a third resection feature configured to guide a cutting tool to form a third osteotomy in the metatarsal, the third resection feature extending through the resection guide from the medial side to the lateral side along a third trajectory at least partially determined based on the bone model. The resection guide also includes a bone attachment feature configured to secure the resection guide to the metatarsal (block 1202).

For example, apparatus may position the resection guide onto a medial surface of a distal end of a first metatarsal 208, as described above.

As also shown in FIG. 12, process 1200 may include deploying a set of fasteners as part of the bone attachment feature to secure the resection guide to the metatarsal (block 1204).

As further shown in FIG. 12, process 1200 may include inserting the cutting tool into the first resection feature, second resection feature, and third resection feature and cutting the metatarsal to form one or more osteotomies of a Reverdin procedure (block 1206). In one embodiment, the Reverdin procedure is a conventional Reverdin procedure. In another embodiment, the Reverdin procedure is a Reverdin-Green procedure. In another embodiment, the Reverdin procedure is a Reverdin-Laird procedure. In another embodiment, the Reverdin procedure is a Reverdin-Todd procedure.

As also shown in FIG. 12, process 1200 may include deploying fixation hardware across one or more of the osteotomies to enable fusion of the metatarsal (block 1208).

Although FIG. 12 shows example blocks of process 1200, in some implementations, process 1200 may include additional blocks, fewer blocks, different blocks, or differently arranged blocks than those depicted in FIG. 12. Additionally, or alternatively, two or more of the blocks of process 1200 may be performed in parallel.

FIGS. 13A-13C illustrate different views of a surgical osteotomy procedure using the osteotomy system of FIG. 9, according to one embodiment. In one embodiment, the surgical osteotomy is a Reverdin procedure.

FIG. 13A illustrates a stage of performing a surgical osteotomy procedure (e.g., Reverdin) using the osteotomy system 900, according to one embodiment. The exemplary osteotomy system 900 can be used to perform osteotomies for a surgical procedure that includes one or more of a Reverdin, a Reverdin-Green, a Reverdin-Todd, and/or a Reverdin-Laird, or the like). A surgeon may elect to approach the osteotomies on the medial side of the foot.

FIG. 13A illustrates a right foot and shows a lateral perspective view of bones around the MTP 806. FIG. 13A includes the medial cuneiform 202, the first metatarsal 208, the first proximal phalange 230, and the first distal phalange 240. FIG. 13A illustrates a resection guide 920 positioned on a medial side of a distal end of the first metatarsal 208. Advantageously, the resection guide 920 includes one or more bone engagement features 926 and/or one or more bone engagement surfaces 928 which assist a surgeon in positioning the resection guide 920 in substantially a same location as a model of the resection guide 920 was placed on a bone model of the first metatarsal 208.

In one embodiment, the surgeon may initially position the resection guide 920 and then translate the resection guide 920 along the bone surface until the bone engagement surface 928 engages and/or interlocks with features on the medial surface of the first metatarsal 208. In certain embodiments, as the resection guide 920 registers to the bone surface, the resection guide 920 may provide tactile feedback to the surgeon to assist with the positioning. For example, if the resection guide 920 can be readily translated, this may be an indication that the resection guide 920 is not yet in the desired position. Similarly, if the resection guide 920 seats or locks into a position, the resection guide 920 may not be readily translatable along the surface and a surgeon can be assured that the resection guide 920 has engaged with the surface features that mirror corresponding features of the bone engagement surface 928 such the resection guide 920 is now in the desired position.

In the illustrated embodiment, the desired position may be on a medial surface just proximal to the head 808 and in a Metaphyseal Diaphyseal Junction or “MDJ”. With the resection guide 920 in a desired position a surgeon is assured that osteotomies made using the resection guide 920 will be in correspondence with those setup in a model and preoperative plan.

FIG. 13B illustrates a stage of performing a surgical osteotomy procedure (e.g., Reverdin) using the osteotomy system 900, according to one embodiment. A surgeon has deployed fasteners 910 into the openings 948 to implement the bone attachment feature(s) 924. The bone attachment feature 924 secure the resection guide 920 to the first metatarsal 208. In one embodiment, the bone attachment features 924 are configured to position, orient, and/or configure one or more openings in the bone for use by fixation fasteners (e.g., bone staples) after the bone attachment features 924 are removed and no longer needed.

The bone attachment features 924 secure the resection guide 920 to the bone for performing an osteotomy. Advantageously, the number, size, configuration, length, width, position, and/or angle in one or more planes of the bone attachment features 924 can be defined for a particular patient or group of patients. In certain embodiments, one or more of the bone attachment features 924 (e.g., bone attachment features 924) may be configured to enter the bone at predefined angles such that the pins, K-wires, or fasteners used as part of the bone attachment features 924 diverge, converge, or are parallel to each other as they extend into the bone.

FIG. 13C illustrates a stage of performing a surgical osteotomy procedure (e.g., Reverdin) using the osteotomy system 900, according to one embodiment. A surgeon has inserted a cutting tool 1310 (e.g., a reciprocating saw blade) into one or more resection features 922 and formed one or more osteotomies of a Reverdin procedure. The bone wedge formed is not show. The distal end of the cutting tool 1310 can be seen stopping short of a lateral cortex 972 of the bone.

FIG. 13D illustrates a closeup view of a resection guide positioned on a metatarsal after forming one or more osteotomies. The resection features 922 guide a cutting tool 1310 in performing one or more osteotomies. Advantageously, as with other embodiments described herein, the number, size, configuration, length, width, position, and/or angle in one or more planes of the resection features 922 can be defined for a particular patient or for a group of patients and can be included in the resection guide 920 provided for a surgical procedure. These aspects of the resection guide 920 may be patient-specific, determined based on patient need, patient anatomy, and/or surgeon preference.

FIG. 13D illustrates a resection guide 920 for a Reverdin-Green wedge osteotomy. The first resection feature 922a and second resection feature 922b extend in parallel from medial side 938 to the lateral side 940. The first resection feature 922a and second resection feature 922b converge on a lateral side of the resection guide 920 to form a wedge. Advantageously, the first trajectory and second trajectory converge at a vertex 950 within the bone (e.g., first metatarsal 208) leaving a lateral cortex 972 intact. In certain embodiments, a surgeon may control the depth of the cutting tool 1310 by way of depth markings on the cutting tool 1310.

Advantageously, the resection guide 920 facilitates forming precision, straight, angled, and/or complex osteotomies for the Reverdin-Green wedge osteotomy procedure. In particular, third resection feature 922c of the resection guide 920 facilitates an osteotomy parallel to a plantar weight bearing surface 1320 of the first metatarsal 208. In addition, the osteotomy formed using the third resection feature 922c is proximal to a distal articular surface 1330 and superior to a plantar aspect 1340 of a head 808 of the first metatarsal 208.

FIG. 13E illustrates a closeup view of one or more osteotomies near a metatarsal head formed using a resection guide 920 of osteotomy system 900 according to one embodiment. The resection guide 920 is omitted for clarity. The resected wedge sections are omitted for clarity. The resection feature 922a enables a surgeon to form a first osteotomy that creates a distal face 1350. The resection feature 922b enables a surgeon to form a second osteotomy that creates a proximal face 1360. The third resection feature 922c enables a surgeon to form a third osteotomy that creates a shelf 1370 (aka a plantar shelf). The third resection feature 922c also provides a plantar stop for the first osteotomy and the second osteotomy. The third resection feature 922c can be positioned and sized to protect against cutting the sesamoids 1380.

In certain embodiments, a surgeon may desire to form the shelf 1370 because the shelf 1370 can help prevent rotation of the head 808 about its long axis. In particular, when the wedge osteotomy is closed (e.g., the distal face 1350 abuts the proximal face 1360) the shelf 1370 contacts and/or abuts a plantar surface 1382 of the neck of the first metatarsal 208. The plantar surface 1382 of the neck of the first metatarsal 208 may be formed by the third osteotomy using the third resection feature 922c. Abutment of the superior surface of the shelf 1370 and the inferior surface of the plantar surface 1382 of the neck of the first metatarsal 208 provide sufficient contact and engagement that the head 808 is not prone to rotate about its long axis.

The plantar surface 1382 is one example of the planar surface on the plantar side of the proximal bone fragment described in relation to the shelf and the second section 1126 and the resection guide 1120 described in relation to FIG. 11A

FIG. 13F illustrates a reduced and fixated distal metatarsal head 8080 after one or more osteotomies using a resection guide 920 according to one embodiment. The wedge osteotomy has been closed such that the distal face 1350 abuts the proximal face 1360 and a superior surface of the shelf 1370 abuts the plantar surface 1382 of the neck of the first metatarsal 208. A surgeon has deployed a medial bone staple 1384 on a medial side of the first metatarsal 208 using the two holes formed by the fasteners 910 used for the osteotomies. In one embodiment, the medial bone staple 1384 may be one of a 15, 16, 18, or 20 mm bone staple (e.g., 12×12, 16×16, or the like).

Alternatively, or in addition, a surgeon has deployed a dorsal bone staple 1386 into holes formed by the surgeon in the dorsal surface of the first metatarsal 208 across the osteotomy. In one embodiment, the dorsal bone staple 1386 may be one of a 15, 16, 18, or 20 mm bone staple (e.g., 12×12, 16×16, or the like). Those of skill in the art will appreciate that other fasteners can be used to fixate the osteotomy besides bone staples.

Those of skill in the art will appreciate that embodiments of the system disclosed herein can be used on humans and animals and on bones that are relatively small in comparison to other bones of the body (e.g., bones of the foot and hand). Advantageously, the embodiments of the system seek to minimize the number of fasteners or pins placed within the bones of a patient by planning a surgical procedure such that pins or fasteners placed in one stage are and/or can be reused in subsequent stages. Consequently, pins initially deployed can remain in the bone or bone fragment as instruments are deployed and/or subsequent stages of the surgical procedure are performed.

Advantageously, because one embodiment uses a bone model of the patient's bones the sizes, dimensions, lengths and configurations of the components of the example systems can each be changed, adapted, revised, and/or customized to meet the needs and/or preferences of the patient and/or surgeon. Advantageously, using the apparatus, systems, and/or methods of the present disclosure the surgeon may have a preoperative plan that identifies which specific bone screw (length, width, diameter, thread, pitch, etc.) to use for the fasteners.

Advantageously, the present disclosure provides an apparatus, system, and/or method that can remediate a condition in a patient's foot. Those of skill in the art will appreciate that the methods, processes, apparatuses, systems, devices, and/or instruments of the present disclosure can be used to address a variety of conditions in a variety of procedures and/or parts of the body of the patient.

Conventionally, correction methods, systems, and/or instrumentation for a condition such as, for example, a bunion and/or a hallux valgus, face several challenges. One example is how to cut the bone such that the cut faces have a desired angle in relation to each other. Advantageously, the present disclosure can address many, if not all, of these challenges to assist a surgeon in performing the surgical procedure and improve the quality of patient care and outcomes.

Any methods disclosed herein comprise one or more steps or actions for performing the described method. The method steps and/or actions may be interchanged with one another. In other words, unless a specific order of steps or actions is required for proper operation of the embodiment, the order and/or use of specific steps and/or actions may be modified.

Reference throughout this specification to “an embodiment” or “the embodiment” means that a particular feature, structure or characteristic described in connection with that embodiment is included in at least one embodiment. Thus, the quoted phrases, or variations thereof, as recited throughout this specification are not necessarily all referring to the same embodiment.

Similarly, it should be appreciated that in the above description of embodiments, various features are sometimes grouped together in a single embodiment, Figure, or description thereof for the purpose of streamlining the disclosure. This method of disclosure, however, is not to be interpreted as reflecting an intention that any claim require more features than those expressly recited in that claim. Rather, as the following claims reflect, inventive aspects lie in a combination of fewer than all features of any single foregoing disclosed embodiment. Thus, the claims following this Detailed Description are hereby expressly incorporated into this Detailed Description, with each claim standing on its own as a separate embodiment. This disclosure includes all permutations of the independent claims with their dependent claims.

Recitation in the claims of the term “first” with respect to a feature or element does not necessarily imply the existence of a second or additional such feature or element. Elements recited in means-plus-function format are intended to be construed in accordance with 35 U.S.C. § 112 Para. 6. It will be apparent to those having skill in the art that changes may be made to the details of the above-described embodiments without departing from the underlying principles set forth herein.

While specific embodiments and applications of the present disclosure have been illustrated and described, it is to be understood that the scope of this disclosure is not limited to the precise configuration and components disclosed herein. Various modifications, changes, and variations which will be apparent to those skilled in the art may be made in the arrangement, operation, and details of the methods and systems of the present disclosure set forth herein without departing from it spirit and scope.

What is claimed is:

1. An apparatus for remediating a bone condition present in a patient's foot, comprising:
a resection guide comprising:
a body having an anterior side, a posterior side, a medial side, a lateral side, a superior side, and an inferior side;
a first resection feature configured to guide a cutting tool to form a first osteotomy in a bone, the first resection feature extending through the resection guide from the medial side to the lateral side along a first trajectory;
a second resection feature configured to guide a cutting tool to form a second osteotomy in the bone, the second resection feature extending through the resection guide from the medial side to the lateral side along a second trajectory;
a third resection feature configured to guide a cutting tool to form a third osteotomy in the bone, the third resection feature extending through the resection guide from the medial side to the lateral side along a third trajectory;
wherein at least one of the first trajectory, the second trajectory, and the third trajectory are at least partially determined based on a bone model of at least a portion of the bone, the bone model based on medical imaging of the patient's foot and configured to resemble an anatomy of the patient's foot; and
a bone attachment feature configured to secure the resection guide to the bone.

2. The apparatus of claim 1, wherein the third resection feature intersects at least one of the first resection feature and the second resection feature at an intersection angle of between about 45 and about 135 degrees.

3. The apparatus of claim 1, wherein the third resection feature intersects both the first resection feature and the second resection feature at an intersection angle of about 90 degrees.

4. The apparatus of claim 1, wherein the first trajectory converges with the second trajectory at a vertex having a wedge angle, and the third trajectory intersects the first trajectory and the second trajectory to form a wedge osteotomy, the wedge angle determined based, at least in part, on the bone model.

5. The apparatus of claim 4, wherein the vertex is prepositioned to be between a lateral cortex of the bone and the resection guide when the resection guide is designed for the bone.

6. The apparatus of claim 5, wherein the vertex is prepositioned such that an osteotomy formed in two of the resection features forms a living hinge at a cortex of the bone.

7. The apparatus of claim 1, wherein the resection guide comprises:
a bone engagement feature configured to engage with at least a portion of the bone at a position that substantially matches a model position of a model of the resection guide engaging the bone model.

8. The apparatus of claim 7, wherein the bone engagement feature comprises:
a bone engagement surface configured to at least partially match a contour of a surface of the bone when the resection guide is positioned for use; and
wherein the bone engagement surface is on one of a lateral side and a medial side of the body.

9. The apparatus of claim 8, wherein the bone engagement surface registers with the surface of the bone abutting the resection guide when the resection guide is positioned for use.

10. The apparatus of claim 1, wherein the bone attachment feature forms at least one hole in the bone, the at least one hole configured to serve as an anchor hole for a fixation device.

11. The apparatus of claim 1, wherein the third resection feature comprises a closed end and an open end.

12. An apparatus for remediating a bone condition present in a patient's foot, comprising:
a resection guide comprising:
a body having an anterior side, a posterior side, a medial side, a lateral side, a superior side, and an inferior side;
a resection feature configured to guide a cutting tool to form a wedge osteotomy in a metatarsal, the wedge osteotomy forming a wedge bone fragment, the wedge osteotomy at least partially determined based on a bone model of at least a portion of the metatarsal; and
a bone attachment feature configured to secure the resection guide to the bone.

13. The apparatus of claim 12, wherein the resection feature is configured to form a shelf in a head of the metatarsal.

14. The apparatus of claim 13, wherein the resection feature is configured to form a planar surface on a proximal side of the wedge osteotomy, the planar surface configured to engage with the shelf during fusion to provide increased bone contact for fusion of the wedge osteotomy.

15. The apparatus of claim 12, wherein the resection guide comprises:
a bone engagement surface configured to at least partially match a contour of a surface of the metatarsal when the resection guide is positioned for use; and
wherein the bone engagement surface is on a lateral side of the body.

16. The apparatus of claim 12, wherein the wedge osteotomy comprises an osteotomy parallel to a plantar weight bearing surface of the metatarsal, proximal to a distal articular surface and superior to a plantar aspect of a metatarsal head of the metatarsal.

17. The apparatus of claim 12, wherein the resection feature comprises:
a first section extending through the resection guide from the medial side to the lateral side, the first section comprising:
a first leg extending through the resection guide along a first trajectory;
a second leg extending through the resection guide along a second trajectory; and
a second section extending through the resection guide from the medial side to the lateral side, the second section intersecting the first leg at a first turn having a first intersection angle and the second leg at a second turn having a second intersection angle, the second section extending through the resection guide along a third trajectory.

18. The apparatus of claim 17, wherein the second section is positioned within the resection guide such that the second section protects sesamoids that are plantar to a head of the metatarsal from dissection.

19. A method for remediating a bone condition present in a patient's foot, the method comprising:
positioning a resection guide onto a medial surface of a distal end of a metatarsal, the resection guide comprising:
a body having an anterior side, a posterior side, a medial side, a lateral side, a superior side, and an inferior side;
a first resection feature configured to guide a cutting tool to form a first osteotomy in a metatarsal, the first resection feature extending through the resection guide from the medial side to the lateral side along a first trajectory at least partially determined based on a bone model of at least a portion of the metatarsal, the bone model based on medical imaging of the patient's foot and configured to resemble an anatomy of the patient's foot;
a second resection feature configured to guide a cutting tool to form a second osteotomy in the metatarsal, the second resection feature extending through the resection guide from the medial side to the lateral side along a second trajectory at least partially determined based on the bone model;
a third resection feature configured to guide a cutting tool to form a third osteotomy in the metatarsal, the third resection feature extending through the resection guide from the medial side to the lateral side along a third trajectory at least partially determined based on the bone model; and
a bone attachment feature configured to secure the resection guide to the metatarsal;
deploying a set of fasteners as part of the bone attachment feature to secure the resection guide to the metatarsal;
inserting the cutting tool into the first resection feature, second resection feature, and third resection feature and cutting the metatarsal to form one or more osteotomies of a Reverdin procedure; and
deploying fixation hardware across one or more of the osteotomies to enable fusion of the metatarsal.

20. The method of claim 19, wherein the Reverdin procedure comprises one of:
a Reverdin-Green procedure,
a Reverdin-Laird procedure; and
a Reverdin-Todd procedure.
